(19) United States
(12) Patent Application Publication
(10) Pub. No.: US 2022/0372474 A1
(43) Pub. Date: Nov. 24, 2022

(54) HYDROXYMETHYL-MODIFIED GAMMA-PNA COMPOSITIONS AND METHODS OF USE THEREOF

(71) Applicant: Yale University, New Haven, CT (US)

(72) Inventors: Elias Quijano, Durham, CT (US); Stanley Oyaghire, New Haven, CT (US); W. Mark Saltzman, New Haven, CT (US); Peter Glazer, Guilford, CT (US)

(21) Appl. No.: 17/620,358

(22) PCT Filed: Jun. 22, 2020
(86) PCT No.: PCT/US2020/038976

§ 371 Date: Dec. 17, 2021

Related U.S. Application Data
(60) Provisional application No. 62/864,984, filed on Jun. 21, 2019.

(51) Int. Cl. C12N 15/113; A61K 9/14

(52) U.S. Cl. CPC C12N 15/113; A61K 9/14; C12N 2310/15; C12N 2310/3181

(57) ABSTRACT

Peptide nucleic acid (PNA) oligomers having one or more hydroxymethyl γ-substitutions, also referred to herein as “^{ser}γPNA”, are provided. The hydroxymethyl γ-substitution preserves and amplifies the helical preorganization that is valuable for DNA duplex invasion by the oligomer. ^{ser}γPNA-containing triplex-forming molecules can be used in combination with a donor DNA fragment to facilitate genome modification in vitro and in vivo.

### CROSS-REFERENCE TO RELATED APPLICATIONS

This application claims the benefit of and priority to U.S. Ser. No. 62/864,984 filed Jun. 21, 2019 and which are incorporated by referenced in their entirety.

### REFERENCE TO SEQUENCE LISTING

The Sequence Listing submitted as a text file named “YU_7622_PCT_ST25.txt,” created on Jun. 22, 2020, and having a size of 39,750 bytes is hereby incorporated by reference pursuant to 37 C.F.R. § 1.52(e)(5).

### STATEMENT REGARDING FEDERALLY SPONSORED RESEARCH

This invention was made with government support under HL125892 awarded by National Institutes of Health. The government has certain rights in the invention.

### FIELD OF THE INVENTION

The field of the invention generally relates to triplex-forming molecules for gene editing and methods of use thereof for ex vivo and in vivo gene editing.

### BACKGROUND OF THE INVENTION

Peptide nucleic acids (PNA) have emerged as useful chemical reagents for effecting gene editing events, in major part because they engage the genome in ways that activate endogenous DNA repair pathways that themselves lead to corrective outcomes through the use of natural or synthetic DNA templates (Rogers, et al., Proceedings of the National Academy of Sciences USA, 99(26):16695-700 (2002). doi: 10.1073/pnas.262556899). Certain PNA oligomers have been shown to activate gene editing events, such as gamma (γ)-tail clamp PNA oligomers, which are PNA variants containing structural modifications leading to improved duplex DNA invasion (e.g., such as the correction of a causal mutation of β-thalassemia; see, e.g., Dragulescu-Andrasi, et al., Journal of the American Chemical Society, 128(31):10258-67. doi: 10.1021/ja0625576 (2006), Bentin, et al., Biochemistry, 42(47):13987-95 (2003). doi: 10.1021/bi0351918, Bahal, et al., Nature Communications, 2016;7:13304 (2016) doi: 10.1038/ncomms13304.). The γPNA utilized in that work specifically featured mini-ethylene glycol as the γ-substitution. The ethylene glycol unit can improve solubility (Sahu, et al., Journal of Organic Chemistry, 76(14):5614-27 (2011) doi: 10.1021/jo200482d.); additionally, γPNA oligomers adopt helical conformations (Dragulescu-Andrasi, et al., Journal of the American Chemical Society, 128(31):10258-67. doi: 10.1021/ja0625576 (2006)) that can enhance DNA binding.

It is nonetheless expedient to explore additional/alternative γPNA variations that circumvent potential syntheses and supply challenges that may be encountered with ^{mp}γPNA monomers and oligomers.

Thus, it is object of the invention to provide alternative γPNA variations.

### SUMMARY OF THE INVENTION

Peptide nucleic acid (PNA) oligomers having one or more hydroxymethyl γ-substitutions, also referred to herein as “^{ser}γPNA”, are provided. The experiments below show that the hydroxymethyl γ-substitution preserves and amplifies the helical preorganization that is valuable for DNA duplex invasion. Polymeric nanoparticles loaded with ^{ser}γPNA-containing triplex-forming molecules and a corrective donor DNA fragment for a thalassemia-associated genotype affected editing correction of the mutation in bone marrow cells derived from thalassemia mouse models at frequencies that were more than double those mediated by ^{mp}γPNA.

The peptide nucleic acid (PNA) oligomers described herein typically include a Hoogsteen binding peptide nucleic acid segment and a Watson-Crick binding PNA segment. Typically, the segments collectively total no more than 50 PNA residues in length, and the two segments can bind or hybridize to a target region comprising a polypurine stretch in a cell's genome to induce strand invasion, displacement, and formation of a triple-stranded molecule among the two PNA segments and the polypurine stretch of the cell's genome. In such embodiments, the number of hydroxymethyl γ-substitution modifications is between 1 and 50, inclusive, PNA residues. Typically, the Hoogsteen binding segment binds to a target nucleic acid duplex by Hoogsteen binding for a length of least five nucleobases, and the Watson-Crick binding segment binds to the target duplex by Watson-Crick binding for a length of least five nucleobases.

In some embodiments, the PNA oligomers, particularly the Hoogsteen binding segment, include one or more chemically modified cytosines selected from the group consisting of pseudocytosine, pseudoisocytosine, and 5-methylcytosine. The Watson-Crick binding segment can include a tail sequence of up to fifteen nucleobases that binds to the target duplex by Watson-Crick binding outside of the triplex. Typically, the two segments are linked by a linker, such as between 1 and 10 units of 8-amino-3,6-dioxaoctanoic acid, 6-aminohexanoic acid, 8-amino-2,6,10-trioxaoctanoic acid, or 11-amino-3,6,9-trioxaundecanoic acid.

In some embodiments, for example, all of the PNA residues in the Hoogsteen binding segment only, in the Watson-Crick binding segment only, or across the entire PNA oligomer are ^{ser}γPNA residues;

one or more of the PNA residues in the Hoogsteen binding segment only or in the Watson-Crick binding segment only of the PNA oligomer or across the entire PNA oligomer are ^{ser}γPNA residues; or

alternating residues in the Hoogsteen binding portion only, in the Watson-Crick binding portion only, or across the entire PNA oligomer are PNA and ^{ser}γPNA residues.

In some embodiments, one or more of the cytosines in the PNA oligomer is replaced with a clamp-G (9-(2-guanidinoethoxy) phenoxazine). The PNA oligomer can have an N-terminus, C-terminus, or both ending with 1, 2, 3 or more lysines.

Preferably, the peptide nucleic acid oligomer can induce a higher frequency of recombination in a population of target cells as a corresponding peptide nucleic acid oligomer wherein the ^{ser}γPNA residues are replaced with mini-PEGγPNA residues or are unmodified.

Pharmaceutical compositions having, for example, an effective amount of the peptide nucleic acid oligomers, are also provided. The composition can include a donor oligonucleotide including a sequence that can correct a mutation(s) in a cell's genome by recombination induced or enhanced by the peptide nucleic acid oligomer. The composition can include nanoparticles, wherein the PNA oligomer, donor oligonucleotide, or a combination thereof are packaged in the same or separate nanoparticles. Exemplary particles include those formed from poly(lactic-co-glycolic acid) (PLGA), poly(beta-amino) esters (PBAEs), blends thereof, e.g., between about 5 and about 25 percent PBAE (wt %). In some embodiments, a targeting moiety, a cell penetrating peptide, or a combination thereof associated with, linked, conjugated, or otherwise attached directly or indirectly to the PNA oligomer or the nanoparticles.

Methods of using the disclosed compositions are also provided. For example, a method of modifying the genome of a cell can include contacting the cell with any of the disclosed oligomers or pharmaceutical compositions. The contacting can occur in vitro or in vivo.

In some in vivo applications, for example, the subject has a genetic disease or disorder caused by a genetic mutation, and the pharmaceutical composition is administered to the subject in an effective amount to correct the mutation in an effective number of cells to reduce one or more symptoms of the disease or disorder. Exemplary genetic diseases or disorder include, but are not limited to, cystic fibrosis, hemophilia, globinopathies such as sickle cell anemia and beta-thalassemia, xeroderma pigmentosum, lysosomal storage diseases, HIV, or cancer.

Any of the methods can further include administering to the subject an effective amount of a potentiating agent to increase the frequency of recombination of the donor oligonucleotide at a target site in the genome of a population of cells.

### BRIEF DESCRIPTION OF THE DRAWINGS

FIG. 1 is a circular dichroism (CD) spectropolarimetry spectra of isosequential PNA oligomers without ethylene glycol (no γ), or with ethylene glycol (mpγ) or hydroxymethyl (serγ) γ-modifications. All samples were prepared in 10 mM NaPi (pH 7.4) buffer.

FIG. 2 is a surface plasmon resonance (SPR) binding sensorgram for hybridization of isosequential PNA oligomers containing no ethylene glycol (noγ), with ethylene glycol (mpγ), or hydroxymethyl (serγ) γ-substitutions to immobilized complementary 80mer DNA oligomer. All samples contained 10 nM of the respective oligo; Running buffer contained 10 mM NaPi (pH 7.4).

FIG. 3A is a bar graph showing nanoparticle diameter as measured by DLS (nm). FIG. 3B is a bar graph showing nanoparticle surface charge. FIG. 3C is a bar graph showing total nucleic acid loading of nanoparticles containing mpγPNA or serγPNA and a correcting donor DNA. All samples were run in triplicate (n=3) **p<0.005.

FIG. 4 is a bar graph showing the results of an ex vivo gene editing assay in primary bone marrow cells derived from a transgenic mouse model of β-thalassemia.

### DETAILED DESCRIPTION OF THE INVENTION

### I. Definitions

As used herein, “transformed” and “transfected” encompass the introduction of a nucleic acid into a cell by one of a number of techniques known in the art.

As used herein, the phrase that a molecule “specifically binds” to a target refers to a binding reaction which is determinative of the presence of the molecule in the presence of a heterogeneous population of other biologics. Thus, under designated assay conditions, a specified molecule binds preferentially to a particular target and does not bind in a significant amount to other biologics present in the sample. Specific binding between two entities can be, for example, an affinity of at least 10⁶, 10⁷, 10⁸, 10⁹, or 10¹⁰ M⁻¹. Affinities greater than 10⁸ M⁻¹ are preferred.

As used herein, the term “carrier” or “excipient” refers to an organic or inorganic ingredient, natural or synthetic inactive ingredient in a formulation, with which one or more active ingredients are combined.

As used herein, the term “pharmaceutically acceptable” means a non-toxic material that does not interfere with the effectiveness of the biological activity of the active ingredients.

As used herein, the terms “effective amount” or “therapeutically effective amount” means a dosage sufficient to alleviate one or more symptoms of a disorder, disease, or condition being treated, or to otherwise provide a desired pharmacologic and/or physiologic effect. The precise dosage will vary according to a variety of factors such as subject-dependent variables (e.g., age, immune system health, etc.), the disease or disorder being treated, as well as the route of administration and the pharmacokinetics of the agent being administered.

As used herein, the term “prevention” or “preventing” means to administer a composition to a subject or a system at risk for or having a predisposition for one or more symptom caused by a disease or disorder to cause cessation of a particular symptom of the disease or disorder, a reduction or prevention of one or more symptoms of the disease or disorder, a reduction in the severity of the disease or disorder, the complete ablation of the disease or disorder, stabilization or delay of the development or progression of the disease or disorder.

The term “subject” or “patient” refers to any mammal who is the target of administration. Thus, the subject can be a human. The subject can be a domesticated, agricultural, or wild animal. Domesticated animals include, for example, dogs, cats, rabbits, ferrets, guinea pigs, hamsters, pigs, monkeys or other primates, and gerbils. Agricultural animals include, for example, horses, cattle, pigs, sheep, rabbits, and goats. The term does not denote a particular age or sex of the subject. In some embodiments, the subject is an embryo or fetus.

### II. Compositions

A. Triplex-forming Molecules

Triplex-forming molecules including peptide nucleic acid (PNA) oligomers with a hydroxymethyl (also referred to as “^{ser}γPNA”) at the gamma position of one or more of the PNA residues are provided.

The triplex forming molecules are typically single stranded and bind to a double stranded nucleic acid molecule, for example duplex DNA, in a sequence-specific manner to form a triple-stranded structure. The single-stranded oligonucleotide/oligomer typically includes a sequence substantially complementary to the polypurine strand of the polypyrimidine:polypurine target motif.

The triplex-forming molecules can be used to induce site-specific homologous recombination in mammalian cells when combined with donor oligonucleotide, e.g., donor DNA molecules. The donor DNA molecules can contain mutated nucleic acids relative to the target DNA sequence. This is useful to activate, inactivate, or otherwise alter the function of a polypeptide or protein encoded by the targeted duplex DNA.

The triplex-forming molecules bind to a predetermined target region referred to herein as the “target sequence,” “target region,” or “target site.” The target sequence for the triplex-forming molecules can be within or adjacent to a human gene encoding, for example, the beta globin, cystic fibrosis transmembrane conductance regulator (CFTR), or an enzyme necessary for the metabolism of a lipid, glycoprotein, or mucopolysaccharide, or another gene in need of correction including those discussed below. The target sequence can be within the coding DNA sequence of the gene or within an intron. The target sequence can also be within DNA sequences which regulate expression of the target gene, including promoter or enhancer sequences or sites that regulate RNA splicing.

Triplex forming molecules are described in more detail below and in U.S. Pat. Nos. 5,962,426, 6,303,376, 7,078,389, 7,279,463, 8,658,608, U.S. Published Application Nos. 2003/0148352, 2010/0172882, 2011/0268810, 2011/0262406, 2011/0293585, and published PCT application numbers WO 1995/001364, WO 1996/040898, WO 1996/039195, WO 2003/052071, WO 2008/086529, WO 2010/123983, WO 2011/053989, WO 2011/133802, WO 2011/13380, WO 2017/143042, WO 2017/143061, WO 2018/187493, Rogers, et al., Proc Natl Acad Sci USA, 99:16695-16700 (2002), Majumdar, et al., Nature Genetics, 20:212-214 (1998), Chin, et al., Proc Natl Acad Sci USA, 105:13514-13519 (2008), and Schleifman, et al., Chem Biol., 18:1189-1198 (2011).

1. Peptide Nucleic Acids

The disclosed triplex forming molecules are formed from peptide nucleic acid (PNA) oligomers with a hydroxymethyl (also referred to as“^{ser}γPNA”) at the gamma position of one or more of the PNA residues (also referred to as PNA monomers).

Peptide nucleic acids are polymeric molecules in which the sugar phosphate backbone of an oligonucleotide has been replaced in its entirety by repeating substituted or unsubstituted N-(2-aminoethyl)-glycine residues that are linked by amide bonds. The various nucleobases are linked to the backbone by methylene carbonyl linkages. PNAs maintain spacing of the nucleobases in a manner that is similar to that of an oligonucleotides (DNA or RNA), but because the sugar phosphate backbone has been replaced, classic (unsubstituted) PNAs are achiral and neutrally charged molecules.

Peptide nucleic acid oligomers are composed of peptide nucleic acid residues (sometimes referred to as ‘residues’). The nucleobases within each PNA residue can include any of the standard bases (uracil, thymine, cytosine, adenine and guanine) or any of the modified heterocyclic nucleobases described below.

a. Gamma Modifications

Some or all of the PNA residues of the disclosed triplex-forming molecules are modified at the gamma position in the polyamide backbone (γPNAs) as illustrated below (wherein “B” is a nucleobase and “R” is a substitution at the gamma position).

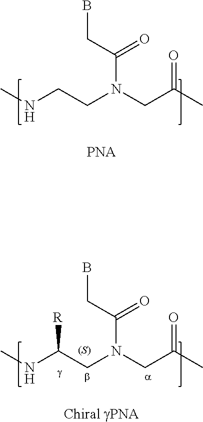

Substitution at the gamma position creates chirality and provides helical pre-organization to the PNA oligomer, yielding substantially increased binding affinity to the target DNA (Rapireddy, et al., Biochemistry, 50(19):3913-8 (2011), He et al., “The Structure of a γ-modified peptide nucleic acid duplex”, Mol. BioSyst. 6:1619-1629 (2010); and Sahu et al., “Synthesis and Characterization of Conformationally Preorganized, (R)-Diethylene Glycol-Containing γ-Peptide Nucleic Acids with Superior Hybridization Properties and Water Solubility”, J. Org. Chem, 76:5614-5627) (2011)). Other advantageous properties can be conferred depending on the chemical nature of the specific substitution at the gamma position (the “R” group in the illustration of the Chiral γPNA, above).

Chemical structures showing substitution at the γ position of the PNA backbone. PNA oligomers have either no (PNA, left), ethylene glycol (mpγPNA, middle), or hydroxymethyl (^{ser}γPNA, right) γ substitution.

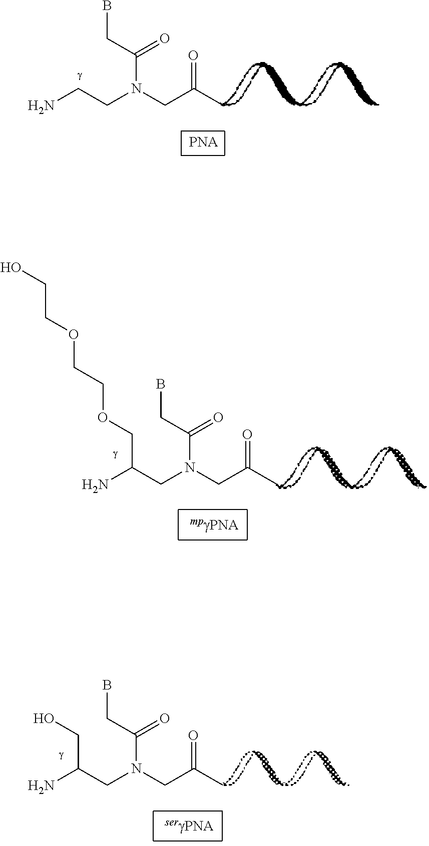

Another class of γ substitution is miniPEG, also depicted above. “MiniPEG” and “MP” refer to diethylene glycol. Other residues and side chains can be considered, and even mixed substitutions can be used to tune the properties of the oligomers.

^{ser}γPNA-containing γPNAs are conformationally preorganized PNAs that may exhibit superior hybridization properties and water solubility as compared to the original PNA design and some other chiral γPNAs.

Sahu et al., describes γPNAs prepared from L-amino acids that adopt a right-handed helix, and γPNAs prepared from D-amino acids that adopt a left-handed helix. Only the right-handed helical γPNAs hybridize to DNA or RNA with high affinity and sequence selectivity.

In some embodiments, tcPNAs are prepared wherein every other PNA residue on the Watson-Crick binding side of the linker is a ^{ser}γPNA-containing γPNA. Accordingly, for these embodiments, the tail clamp side of the PNA has alternating classic PNA and ^{ser}γPNA-containing γPNA residues.

In some embodiments, PNA-mediated gene editing are achieved via additional or alternative γ substitutions or other PNA chemical modifications including but limited to those introduced above and below. Examples of γ substitution with other side chains include that of alanine, threonine, cysteine, valine, leucine, isoleucine, methionine, proline, phenylalanine, tyrosine, aspartic acid, glutamic acid, asparagine, glutamine, histidine, lysine, arginine, and the derivatives thereof. The “derivatives thereof” as used herein are defined as those chemical moieties that are covalently attached to these amino acid side chains, for instance, to that of serine, cysteine, threonine, tyrosine, aspartic acid, glutamic acid, asparagine, glutamine, lysine, and arginine.

b. Additional PNA Modifications

PNA oligomers can also include other positively charged moieties to increase the solubility of the PNA and increase the affinity of the PNA for duplex DNA. Commonly used positively charged moieties include the amino acids lysine and arginine, although other positively charged moieties may also be useful. Lysine and arginine residues can be added to a bis-PNA linker or can be added to the carboxy or the N-terminus of a PNA strand. Common modifications to PNA oligomers are discussed in Sugiyama and Kittaka, Molecules, 18:287-310 (2013)) and Sahu, et al., J. Org. Chem., 76, 5614-5627 (2011), each of which are specifically incorporated by reference in their entireties, and include, but are not limited to, incorporation of charged amino acid residues, such as lysine at the termini or in the interior part of the oligomer; inclusion of polar groups in the backbone, a carboxymethylene bridge in the nucleobases; chiral PNA oligomers bearing substituents on the original N-(2-aminoethyl)glycine backbone; replacement of the original aminoethylglycyl backbone skeleton with a negatively-charged scaffold; conjugation of high molecular weight polyethylene glycol (PEG) to one of the termini; fusion of a PNA oligomer to DNA to generate a chimeric oligomer, redesign of the backbone architecture, conjugation of PNA to DNA or RNA. These modifications improve solubility but often result in reduced binding affinity and/or sequence specificity.

Additionally, any of the triplex-forming sequences can be modified to include guanidine-G-clamp (“G-clamp”) PNA residues(s) to enhance PNA oligomer binding to a target site, wherein the G-clamp is linked to the backbone as any other nucleobase would be. γPNAs with substitution of cytosine by G-clamp (9-(2-guanidinoethoxy) phenoxazine), a cytosine analog that can form five H-bonds with guanine, and can also provide extra base stacking due to the expanded phenoxazine ring system and substantially increased binding affinity. In vitro studies indicate that a single G-clamp substitution for C can substantially enhance the binding of a PNA-DNA duplex by 23 C (Kuhn, et al., Artificial DNA, PNA & XNA, 1(1):45-53(2010)). As a result, γPNAs containing G-clamp substitutions can have further increased activity.

The structure of a G-clamp monomer-to-G base pair (G-clamp indicated by the “X”) is illustrated below in comparison to C-G base pair.

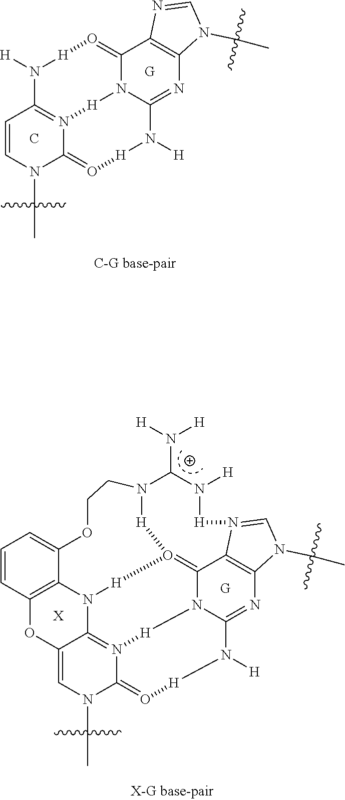

Some studies have shown improvements using D-amino acids in peptide synthesis.

In some embodiments, the PNA oligomer includes a Hoogsteen binding peptide nucleic acid (PNA) segment and a Watson-Crick binding PNA segment collectively totaling no more than 50 nucleobases in length, wherein the two segments bind or hybridize to a target region of a genomic DNA comprising a polypurine stretch to induce strand invasion, displacement, and formation of a triple-stranded composition among the two PNA segments and the polypurine stretch of the genomic DNA, wherein the Hoogsteen binding segment binds to the target region by Hoogsteen binding for a length of least five nucleobases, and wherein the Watson-Crick binding segment binds to the target region by Watson-Crick binding for a length of least five nucleobases.

The Hoogsteen binding segment can include one or more chemically modified cytosines selected from the group consisting of pseudocytosine, pseudoisocytosine, and 5-methylcytosine. The Watson-Crick binding segment can include a sequence of up to fifteen nucleobases that binds to the target duplex by Watson-Crick binding outside of the triplex. The two segments can be linked by a linker. In some embodiments, all of the peptide nucleic acid residues in the Hoogsteen binding segment only, in the Watson-Crick binding segment only, or across the entire PNA oligomer include a gamma modification of a backbone carbon. In some embodiments, one or more of the peptide nucleic acid residues in the Hoogsteen binding segment only or in the Watson-Crick binding segment only of the PNA oligomer include a gamma modification of a backbone carbon. In some embodiments, alternating peptide nucleic acid residues in the Hoogsteen binding portion only, in the Watson-Crick binding portion only, or across the entire PNA oligomer include a gamma modification of a backbone carbon.

Typically, least one gamma modification of the backbone carbon is a gamma hydroxymethyl modification. In some embodiments, all gamma modifications are gamma hydroxymethyl modifications. Optionally, at least one PNA segment includes a G-clamp (9-(2-guanidinoethoxy) phenoxazine).

2. Form of the Triplex-Forming Molecules

a. Triplex-Forming Oligonucleotides (TFOs)

Triplex-forming oligonucleotides (TFOs) are defined as oligonucleotides which bind as third strands to duplex DNA in a sequence specific manner As introduced above, the disclosed triplex forming oligonucleotides are composed of PNA oligomers, wherein at least one of the PNA residues has a hydroxymethyl (also referred to as “^{ser}γPNA”) at the gamma position. The oligonucleotides are synthetic or isolated nucleic acid molecules which selectively bind to or hybridize with a predetermined target sequence, target region, or target site within or adjacent to a human gene so as to form a triple-stranded structure.

Preferably, the oligonucleotide is a single-stranded peptide nucleic acid molecule between 7 and 40 nucleotides in length, most preferably 10 to 20 nucleotides in length for in vitro mutagenesis and 20 to 30 nucleotides in length for in vivo mutagenesis. The nucleobase (sometimes referred to herein simply as “base”) composition in the oligonucleotide may be homopurine or homopyrimidine. Alternatively, the nucleobase composition in the oligonucleotide may be polypurine or polypyrimidine. However, other compositions are also useful.

The nucleobase sequence of the oligonucleotides/oligomer is selected based on the sequence of the target sequence, the physical constraints imposed by the need to achieve binding of the oligonucleotide/oligomer within the major groove of the target region, and the need to have a low dissociation constant (K_{d}) for the oligo/target sequence complex. The oligonucleotides/oligomers have a nucleobase composition which is conducive to triple-helix formation and is generated based on one of the known structural motifs for third strand binding (e.g. Hoogsteen binding). Stable complexes are often formed on polypurine:polypyrimidine elements, which are relatively abundant in mammalian genomes. Triplex formation by TFOs can occur with the third strand oriented either parallel or anti-parallel to the purine strand of the nucleic acid duplex. In the anti-parallel, purine motif, the triplets are G.G:C and A.A:T, whereas in the parallel pyrimidine motif, the canonical triplets are C⁺.G:C and T.A:T. The triplex structures can be stabilized by one, two or three Hoogsteen hydrogen bonds (depending on the nucleobase) between the bases in the TFO strand and the purine strand in the duplex. A review of base compositions and binding properties for third strand binding oligonucleotides and/or peptide nucleic acids is provided in, for example, U.S. Pat. No. 5,422,251, Bentin et al., Nucl. Acids Res., 34(20): 5790-5799 (2006), and Hansen et al., Nucl. Acids Res., 37(13): 4498-4507 (2009).

Preferably, the oligonucleotide/oligomer binds to or hybridizes to the target sequence under conditions of high stringency and specificity. In some embodiments, the oligonucleotides/oligomers bind in a sequence-specific manner within the major groove of duplex DNA. Reaction conditions for in vitro triple helix formation of an oligonucleotide/oligomer to a double stranded nucleic acid sequence vary from oligo to oligo, depending on factors such as polymer length, the number of G:C and A:T base pairs, and the composition of the buffer utilized in the hybridization reaction. An oligonucleotide substantially complementary, based on the third strand binding code, to the target region of the double-stranded nucleic acid molecule is preferred.

As used herein, a triplex forming molecule is said to be substantially complementary to a target region when the oligonucleotide has a nucleobase composition which allows for the formation of a triple-helix with the target region. As such, an oligonucleotide/oligomer can be substantially complementary to a target region even when there are non-complementary bases present in the oligonucleotide/oligomer. As stated above, there are a variety of structural motifs available which can be used to determine the nucleobase sequence of a substantially complementary oligonucleotide/oligomer.

PNAs can bind to DNA via Watson-Crick hydrogen bonds, but with binding affinities significantly higher than those of a corresponding nucleotide composed of DNA or RNA. The neutral backbone of PNAs decreases electrostatic repulsion between the PNA and target DNA phosphates. Under in vitro or in vivo conditions that promote opening of the duplex DNA, PNAs can mediate strand invasion of duplex DNA resulting in displacement of one DNA strand to form a D-loop.

Highly stable triplex PNA:DNA:PNA structures can be formed from a homopurine DNA strand and two PNA strands. The two PNA strands may be two separate PNA molecules (see Bentin et al., Nucl. Acids Res., 34(20): 5790-5799 (2006) and Hansen et al., Nucl. Acids Res., 37(13): 4498-4507 (2009)), or two PNA molecules linked together by a linker of sufficient flexibility to form a single bis-PNA molecule (See: U.S. Pat. No. 6,441,130). In both cases, the PNA molecule(s) forms a triplex “clamp” with one of the strands of the target duplex while displacing the other strand of the duplex target. In this structure, one strand forms Watson-Crick base pairs with the DNA strand in the anti-parallel orientation (the Watson-Crick binding portion), whereas the other strand forms Hoogsteen base pairs to the DNA strand in the parallel orientation (the Hoogsteen binding portion). A homopurine strand allows formation of a stable PNA/DNA/PNA triplex. PNA clamps can form at shorter homopurine sequences than those required by triplex-forming oligonucleotides (TFOs) and also do so with greater stability.

Suitable molecules for use in linkers of bis-PNA molecules include, but are not limited to, 8-amino-3,6-dioxaoctanoic acid, referred to as an O-linker, and 6-aminohexanoic acid. Poly(ethylene) glycol monomers can also be used in bis-PNA linkers. A bis-PNA linker can contain multiple linker residues in any combination of two or more of the foregoing.

PNAs can also include other positively charged moieties to increase the solubility of the PNA and increase the affinity of the PNA for duplex DNA. Commonly used positively charged moieties include the amino acids lysine and arginine (e.g., as additional substituents attached to the C- or N-terminus of the PNA oligomer (or a segment thereof) or as a side-chain modification of the backbone (see Huang et al., Arch. Pharm. Res. 35(3): 517-522 (2012) and Jain et al., JOC, 79(20): 9567-9577 (2014)), although other positively charged moieties may also be useful (See for Example: U.S. Pat. No. 6,326,479). In some embodiments, the PNA oligomer can have one or more ‘miniPEG’ side chain modifications of the backbone (see, for example, U.S. Pat. No. 9,193,758 and Sahu et al., JOC, 76: 5614-5627 (2011)).

Peptide nucleic acids are unnatural synthetic polyamides that can beprepared using known methodologies, generally as adapted from peptide synthesis processes.

b. Clamps and Tail Clamps

Some triplex-forming molecules, such as PNA oligomer clamps and tail clamp PNAs (tcPNAs) invade the target nucleic acid duplex, with displacement of the polypyrimidine strand, and induce triplex formation with the polypurine strand of the target duplex by both Watson-Crick and Hoogsteen binding. Preferably, both the Watson-Crick and Hoogsteen binding portions of the triplex forming molecules are substantially complementary to the target sequence. Although, as with triplex-forming oligonucleotides, a homopurine strand is needed to allow formation of a stable PNA/DNA/PNA triplex, PNA clamps can form at shorter homopurine sequences than those required by triplex-forming oligonucleotides and also do so with greater stability.

Preferably, PNAs are between 6 and 50 nucleobase-containing residues in length. The Watson-Crick portion should be 9 or more nucleobase-containing residues in length, optionally including a tail sequence. More preferably, the Watson-Crick binding portion is between about 9 and 30 nucleobase-containing residues in length, optionally including a tail sequence of between 0 and about 15 nucleobase-containing residues. More preferably, the Watson-Crick binding portion is between about 10 and 25 nucleobase-containing residues in length, optionally including a tail sequence of between 0 and about 10 nucleobase-containing residues in length. In a preferred embodiment, the Watson-Crick binding portion is between 15 and 25 nucleobase-containing residues in length, optionally including a tail sequence of between 5 and 10 nucleobase-containing residues in length. The Hoogsteen binding portion should be 6 or more nucleobase residues in length. Most preferably, the Hoogsteen binding portion is between about 6 and 15 nucleobase-containing residues in length, inclusive.

Although polypurine:polypyrimidine stretches do exist in mammalian genomes, it is desirable to target triplex formation in the absence of this requirement. In some embodiments, triplex-forming molecules include a “tail” added to the end of the Watson-Crick binding portion. Adding additional nucleobases, known as a “tail” or “tail clamp” or “tc”, to the Watson-Crick binding portion that bind to the target strand outside the triple helix further reduces the requirement for a polypurine:polypyrimidine stretch and increases the number of potential target sites.

The tail is most typically added to the end of the Watson-Crick binding sequence furthest from the linker. This molecule therefore mediates a mode of binding to DNA that encompasses both triplex and duplex formation (Kaihatsu, et al., Biochemistry, 42(47):13996-4003 (2003); Bentin, et al., Biochemistry, 42(47):13987-95 (2003)). For example, if the triplex-forming molecules are tail clamp PNA (tcPNA), the PNA/DNA/PNA triple helix portion and the PNA/DNA duplex portion both produce displacement of the pyrimidine-rich strand, creating an altered helical structure that strongly provokes the nucleotide excision repair pathway and activating the site for recombination with a donor DNA molecule (Rogers, et al., Proc. Natl. Acad. Sci. U.S.A., 99(26):16695-700 (2002)).

Tails added to clamp PNAs (sometimes referred to as bis-PNAs) form tail-clamp PNAs (referred to as tcPNAs) that have been described by Kaihatsu, et al., Biochemistry, 42(47):13996-4003 (2003); Bentin, et al., Biochemistry, 42(47):13987-95 (2003). tcPNAs are known to bind to DNA more efficiently due to low dissociation constants. The addition of the tail also increases binding specificity and binding stringency of the triplex-forming molecules to the target duplex. It has also been found that the addition of a tail to clamp PNA improves the frequency of recombination of the donor oligonucleotide at the target site compared to PNA without the tail.

Traditional nucleic acid TFOs may need a stretch of at least 15 and preferably 30 or more nucleobase-containing residues. Peptide nucleic acids need fewer purines to a form a triple helix, although typically at least 10 or preferably more may be needed. Peptide nucleic acids including a tail, also referred to tail clamp PNAs, or tcPNAs, require even fewer purines to a form a triple helix. A triple helix may be formed with a target sequence containing fewer than 8 purines. Therefore, PNAs should be designed to target a site on duplex nucleic acid containing between 6-30 polypurine:polypyrimidines, preferably, 6-25 polypurine:polypyrimidines, more preferably 6-20 polypurine:polypyrimidines.

The addition of a “mixed-sequence” tail to the Watson-Crick binding strand of the triplex-forming molecules such as PNAs also increases the length of the triplex-forming molecule and, correspondingly, the length of the binding site. This increases the target specificity and size of the lesion created at the target site and disrupts the helix in the duplex nucleic acid, while maintaining a low requirement for a stretch of polypurine:polypyrimidines. Increasing the length of the target sequence improves specificity for the target, for example, a target of 17 base pairs will statistically be unique in the human genome. Relative to a smaller lesion, it is likely that a larger triplex lesion with greater disruption of the underlying DNA duplex will be detected and processed more quickly and efficiently by the endogenous DNA repair machinery that facilitates recombination of the donor oligonucleotide.

In some embodiments a PNA tail clamp system includes:

a) optionally, a positively charged region having a positively charged amino acid subunit, e.g., a lysine subunit;

b) a first region including a plurality of PNA subunits having Hoogsteen homology with a target sequence;

c) a second region including a plurality of PNA subunits having Watson Crick homology binding with the target sequence;

d) a third region including a plurality of PNA subunits having Watson Crick homology binding with a tail target sequence;

e) optionally, a second positively charged region having a positively charged amino acid subunit, e.g., a lysine subunit.

In some embodiments, a linker is disposed between b) and c). In some embodiments, one or more PNA residues of the tail clamp is modified as disclosed herein.

B. Donor Oligonucleotides

In some embodiments, the composition includes or is administered in combination with a donor oligonucleotide. The donor oligonucleotide can be encapsulated or entrapped in the same or different particles from other active agents such as the triplex forming composition. Generally, in the case of gene therapy, the donor oligonucleotide includes a sequence that can correct a mutation(s) in the host genome, though in some embodiments, the donor introduces a mutation that can, for example, reduce expression of an oncogene or a receptor that facilitates HIV infection. In addition to containing a sequence designed to introduce the desired correction or mutation, the donor oligonucleotide may also contain synonymous (silent) mutations (e.g., 7 to 10). The additional silent mutations can facilitate detection of the corrected target sequence using allele-specific PCR of genomic DNA isolated from treated cells. Triplex-forming composition and other gene editing compositions such as those discussed above can increase the rate of recombination of the donor oligonucleotide in the target cells relative to administering donor alone.

The triplex forming molecules including peptide nucleic acids may be administered in combination with, or tethered to, a donor oligonucleotide via a mixed sequence linker or used in conjunction with a non-tethered donor oligonucleotide that is substantially homologous to the target sequence. Triplex-forming molecules can induce recombination of a donor oligonucleotide sequence up to several hundred base pairs away. It is preferred that the donor oligonucleotide sequence targets a region between 0 to 800 bases from the target binding site of the triplex-forming molecules. In some embodiments, the donor oligonucleotide sequence targets a region between 25 to 75 bases from the target binding site of the triplex-forming molecules. In some embodiments, the donor oligonucleotide sequence targets a region about 50 nucleotides from the target binding site of the triplex-forming molecules.

The donor sequence can contain one or more nucleic acid sequence alterations compared to the sequence of the region targeted for recombination, for example, a substitution, a deletion, or an insertion of one or more nucleotides. Successful recombination of the donor sequence results in a change of the sequence of the target region. Donor oligonucleotides are also referred to herein as donor fragments, donor nucleic acids, donor DNA, or donor DNA fragments. This strategy exploits the ability of a triplex to provoke DNA repair, potentially increasing the probability of recombination with the homologous donor DNA. It is understood in the art that a greater number of homologous positions within the donor fragment will increase the probability that the donor fragment will be recombined into the target sequence, target region, or target site. Tethering of a donor oligonucleotide to a triplex-forming molecule facilitates target site recognition via triple helix formation while at the same time positioning the tethered donor fragment for possible recombination and information transfer. Triplex-forming molecules also effectively induce homologous recombination of non-tethered donor oligonucleotides. The term “recombinagenic” as used herein, is used to define a DNA fragment, oligonucleotide, peptide nucleic acid, or composition as being able to recombine into a target site or sequence or induce recombination of another DNA fragment, oligonucleotide, or composition.

Non-tethered or unlinked fragments may range in length from 20 nucleotides to several thousand. The donor oligonucleotide molecules, whether linked or unlinked, can exist in single stranded or double stranded form. The donor fragment to be recombined can be linked or un-linked to the triplex forming molecules. The linked donor fragment may range in length from 4 nucleotides to 100 nucleotides, preferably from 4 to 80 nucleotides in length. However, the unlinked donor fragments have a much broader range, from 20 nucleotides to several thousand. In one embodiment the oligonucleotide donor is between 25 and 80 nucleobases. In a further embodiment, the non-tethered donor oligonucleotide is about 50 to 60 nucleotides in length.

The donor oligonucleotides may contain at least one mutated, inserted or deleted nucleotide relative to the target DNA sequence. Target sequences can be within the coding DNA sequence of the gene or within introns. Target sequences can also be within DNA sequences which regulate expression of the target gene, including promoter or enhancer sequences or sequences that regulate RNA splicing.

The donor oligonucleotides can contain a variety of mutations relative to the target sequence. Representative types of mutations include, but are not limited to, point mutations, deletions and insertions. Deletions and insertions can result in frameshift mutations or deletions. Point mutations can cause missense or nonsense mutations. These mutations may disrupt, reduce, stop, increase, improve, or otherwise alter the expression of the target gene.

Compositions including triplex-forming molecules such as tcPNA may include one or more than one donor oligonucleotides. More than one donor oligonucleotides may be administered with triplex-forming molecules in a single transfection, or sequential transfections. Use of more than one donor oligonucleotide may be useful, for example, to create a heterozygous target gene where the two alleles contain different modifications.

Donor oligonucleotides are preferably DNA oligonucleotides, composed of the principal naturally-occurring nucleotides (uracil, thymine, cytosine, adenine and guanine) as the heterocyclic nucleobases, deoxyribose as the sugar moiety, and phosphate ester linkages. Donor oligonucleotides may include modifications to nucleobases, sugar moieties, or backbone/linkages, as described above, depending on the desired structure of the replacement sequence at the site of recombination or to provide some resistance to degradation by nucleases. One exemplary modification is a thiophosphate ester linkage. Modifications to the donor oligonucleotide should not prevent the donor oligonucleotide from successfully recombining at the recombination target sequence in the presence of triplex-forming molecules.

C. Nucleobase, Sugar, and Linkage Modifications

Any of the triplex-forming molecules, components thereof, donor oligonucleotides, or other nucleic acids disclosed herein can include one or more modifications or substitutions to the nucleobases or linkages. Although modifications are particularly preferred for use with triplex-forming technologies and typically discussed below with reference thereto, any of the modifications can be utilized in the construction of any of the gene editing compositions, donor, nucleotides, etc. Modifications should not prevent, but preferably enhance the activity, persistence, or function of the gene editing technology. For example, modifications to oligonucleotides for use as triplex-forming molecules should not prevent, but preferably enhance duplex invasion, strand displacement, and/or stabilize triplex formation as described above by increasing specificity or binding affinity of the triplex-forming molecules to the target site. Modified bases and base analogues, modified sugars and sugar analogues and/or various suitable linkages known in the art are also suitable for use in the molecules disclosed herein. Several preferred oligonucleotide compositions including PNA, and modification thereof to include ser at the γ position in the PNA backbone, are discussed above. Additional modifications are discussed in more detail below.

1. Nucleobases

The principal naturally-occurring nucleotides include uracil, thymine, cytosine, adenine and guanine as the heterocyclic nucleobases. Gene editing molecules can include chemical modifications to their nucleotide constituents. For example, target sequences with adjacent cytosines can be problematic. Triplex stability is greatly compromised by runs of cytosines, thought to be due to repulsion between the positive charge resulting from the N³ protonation or perhaps because of competition for protons by the adjacent cytosines. Chemical modification of nucleotides including triplex-forming molecules such as PNAs may be useful to increase binding affinity of triplex-forming molecules and/or triplex stability under physiologic conditions.

Chemical modifications of nucleobases or nucleobase analogs may be effective to increase the binding affinity of a nucleotide or its stability in a triplex. Chemically-modified nucleobases include, but are not limited to, inosine, 5-(1-propynyl) uracil (pU), 2-thio uracil, 5-(1-propynyl) cytosine (pC), 5-methylcytosine, 8-oxo-adenine, 2,6-diaminopurine, pseudocytosine, pseudoisocytosine, 5 and 2-amino-5-(2′-deoxy-β-D-ribofuranosyl)pyridine (2-aminopyridine), and various pyrrolo- and pyrazolopyrimidine derivatives. Substitution of 5-methylcytosine or pseudoisocytosine for cytosine in triplex-forming molecules such as PNAs helps to stabilize triplex formation at neutral and/or physiological pH, especially in triplex-forming molecules with isolated cytosines.

2. Backbone

The nucleotide residues of the triplex-forming molecules are connected by an internucleotide bond that refers to a chemical linkage between two nucleoside moieties. Unmodified peptide nucleic acids (PNAs) are synthetic DNA mimics in which the phosphate backbone of the oligonucleotide is replaced in its entirety by repeating N-(2-aminoethyl)-glycine units that are linked by amide bonds. The various nucleobases are linked to the backbone by methylene carbonyl bonds, which allow them to form PNA-DNA or PNA-RNA duplexes via Watson-Crick base pairing with high affinity and sequence-specificity. PNAs maintain spacing of nucleobases that is similar to conventional DNA oligonucleotides, but are achiral and neutrally charged molecules. Peptide nucleic acids are composed of peptide nucleic acid residues.

Other backbone modifications, particularly those relating to PNAs, include peptide and amino acid variations and modifications. Thus, the backbone constituents of PNAs may be peptide linkages, or alternatively, they may be non-peptide linkages. Examples include acetyl caps, amino spacers such as 8-amino-3,6-dioxaoctanoic acid (referred to herein as O-linkers), amino acids such as lysine are particularly useful if positive charges are desired in the PNA, and the like. Methods for the chemical assembly of PNAs are well known. See, for example, U.S. Pat. Nos. 5,539,082, 5,527,675, 5,623,049, 5,714,331, 5,736,336, 5,773,571 and 5,786,571.

Backbone modifications used to generate triplex-forming molecules should not prevent the molecules from binding with high specificity to the target site and creating a triplex with the target duplex nucleic acid by displacing one strand of the target duplex and forming a clamp around the other strand of the target duplex.

3. Modified Nucleic Acids

Modified nucleic acids in addition to peptide nucleic acids are also useful as triplex-forming molecules. Oligonucleotides are composed a chain of nucleotides which are linked to one another. Canonical nucleotides typically are composed of a nucleobase (nucleic acid base), a sugar moiety attached to the heterocyclic base, and a phosphate moiety which esterifies a hydroxyl function of the sugar moiety. The principal naturally-occurring nucleotides include uracil, thymine, cytosine, adenine and guanine as the heterocyclic nucleobases, and ribose or deoxyribose sugar linked by phosphodiester bonds. As used herein “modified nucleotide” or “chemically modified nucleotide” defines a nucleotide that has a chemical modification of one or more of the nucleobase, sugar moiety or phosphate moiety constituents. Preferably the charge of the modified nucleotide is reduced compared to DNA or RNA oligonucleotides of the same nucleobase sequence. Most preferably the triplex-forming molecules have low negative charge, no charge, or positive charge such that electrostatic repulsion with the nucleotide duplex at the target site is reduced compared to DNA or RNA oligonucleotides with the corresponding nucleobase sequence.

Examples of modified nucleotides with reduced charge include modified internucleotide linkages such as phosphate analogs having achiral and uncharged intersubunit linkages (e.g., Sterchak, E. P. et al., Organic Chem., 52:4202, (1987)), and uncharged morpholino-based polymers having achiral intersubunit linkages (see, e.g., U.S. Pat. No. 5,034,506). Some internucleotide linkage analogs include morpholidate, acetal, and polyamide-linked heterocycles. Locked nucleic acids (LNA) are modified RNA nucleotides (see, for example, Braasch, et al., Chem. Biol., 8(1):1-7 (2001)). LNAs form hybrids with DNA which are more stable than DNA/DNA hybrids, a property similar to that of peptide nucleic acid (PNA)/DNA hybrids. Therefore, LNA can be used just as PNA molecules would be except they have a negatively charged backbone, whereas PNAs generally have a neutrally charged backbone (although certain amino acid side chain modifications can alter the backbone charge). LNA binding efficiency can be increased in some embodiments by adding positive charges to it. Commercial nucleic acid synthesizers and standard phosphoramidite chemistry can be used to make LNAs.

Molecules may also include nucleotides with modified nucleobases, sugar moieties or sugar moiety analogs. Modified nucleotides may include modified nucleobases or base analogs as described above with respect to peptide nucleic acids. Sugar moiety modifications include, but are not limited to, 2′-O-aminoethoxy, 2′-O-amonioethyl (2′-OAE), 2′-O-methoxy, 2′-O-methyl, 2-guanidoethyl (2′-OGE), 2′-0,4′-C-methylene (LNA), 2′-O-(methoxyethyl) (2′-OME) and 2′-O-(N-(methyl)acetamido) (2′-OMA). 2′-O-aminoethyl sugar moiety substitutions are especially preferred because they are protonated at neutral pH and thus suppress the charge repulsion between the triplex-forming molecule and the target duplex. This modification stabilizes the C3′-endo conformation of the ribose or deoxyribose and also forms a bridge with the i-1 phosphate in the purine strand of the duplex.

D. Gene Editing Potentiating Factors

In some embodiments, the compositions and methods include a potentiating factor.

Accordingly, compositions and methods of increasing the efficacy of gene editing technology are provided. As used herein a “gene editing potentiating factor” or “gene editing potentiating agent” or “potentiating factor or “potentiating agent” refers a compound that increases the efficacy of editing (e.g., mutation, including insertion, deletion, substitution, etc.) of a gene, genome, or other nucleic acid) by a gene editing technology relative to use of the gene editing technology in the absence of the compound. Preferred gene editing technologies suitable for use alone or more preferably in combination with the potentiating factors are discussed in more detail below. In some embodiments, the potentiating factor is administered as a nucleic acid encoding the potentiating factor. In certain preferred embodiments, the gene editing technology is a triplex-forming γPNA and donor DNA, optionally, but preferably in a particle composition.

Potentiating factors include, for example, DNA damage or repair-stimulating or -potentiating factors. Preferably the factor is one that engages one or more endogenous high fidelity DNA repair pathways. In some embodiments, the factor is one that modulates expression of Rad51, BRCA2, or a combination thereof.

As discussed in more detail below, the preferred methods typically include contacting cells with an effective amount of a gene editing potentiating factor. The contacting can occur ex vivo, for example isolated cells, or in vivo following, for example, administration of the potentiating factor to a subject. Examplary gene editing potentiating agents include receptor tyrosine kinase C-kit ligands, ATR-Chk1 cell cycle checkpoint pathway inhibitors, a DNA polymerase alpha inhibitors, and heat shock protein 90 inhibitors (HSP90i).

In some embodiments, the C-kit ligand is stem factor protein or fragment thereof sufficient to causes dimerization of C-kit and activates its tyrosine kinase activity. The C-kit ligand can be a nucleic acid encoding a stem cell factor (SCF) protein or fragment thereof sufficient to causes dimerization of C-kit and activates its tyrosine kinase activity. The nucleic acid can be an mRNA or an expression vector. The SCF can be human SCF or a fragment or variant thereof.

In some embodiments, the potentiating agent is another cytokine or growth factor such as, erythropoietin, GM-CSF, EGF (especially for epithelial cells; lung epithelia for cystic fibrosis), hepatocyte growth factor etc., could similarly serve to boost gene editing potential in bone marrow cells or in other tissues. In some embodiments, gene editing is enhanced in specific cell types using cytokines targeted to these cell types.

It will be appreciated that cytokines and growth factors including SCF can be administered to cells or a subject as protein, or as a nucleic acid encoding protein (transcribed RNA, DNA, DNA in an expression vector). For example, a sequence encoding a protein or growth factor such as SCF can be incorporated into an autonomously replicating plasmid, a virus (e.g., a retrovirus, lentivirus, adenovirus, or herpes virus), or into the genomic DNA of a prokaryote or eukaryote.

In some embodiments, the potentiating factor is a replication modulator that can, for example, manipulate replication progression and/or replication forks. For example, the ATR-Chk1 cell cycle checkpoint pathway has numerous roles in protecting cells from DNA damage and stalled replication, one of the most prominent being control of the cell cycle and prevention of premature entry into mitosis (Thompson and Eastman, Br J Clin Pharmacol., 76(3): 358-369 (2013), Smith, et al., Adv Cancer Res., 108:73-112 (2010)). However, Chk1 also contributes to the stabilization of stalled replication forks, the control of replication origin firing and replication fork progression, and homologous recombination. DNA polymerase alpha also known as Pol α is an enzyme complex found in eukaryotes that is involved in initiation of DNA replication. Hsp90 (heat shock protein 90) is a chaperone protein that assists other proteins to fold properly, stabilizes proteins against heat stress, and aids in protein degradation.

Experimental results show that inhibitors of CHK1 and ATR in the DNA damage response pathway, as well as DNA polymerase alpha inhibitors and HSP90 inhibitors, substantially boost gene editing by triplex-forming PNAs and single-stranded donor DNA oligonucleotides. Accordingly, in some embodiments, the potentiating factor is a CHK1 or ATR pathway inhibitor, a DNA polymerase alpha inhibitor, or an HSP90 inhibitor. The inhibitor can be a functional nucleic acid, for example siRNA, miRNA, aptamers, ribozymes, triplex forming molecules, RNAi, or external guide sequences that targets CHK1, ATR, or another molecule in the ATR-Chk1 cell cycle checkpoint pathway; DNA polymerase alpha; or HSP90 and reduces expression or active of ATR, CHK1, DNA polymerase alpha, or HSP90.

Preferably, the inhibitor is a small molecule. For example, the potentiating factor can be a small molecule inhibitor of ATR-Chk1 Cell Cycle Checkpoint Pathway Inhibitor. Such inhibitors are known in the art, and many have been tested in clinical trials for the treatment of cancer. Exemplary CHK1 inhibitors include, but are not limited to, AZD7762, SCH900776/ MK-8776, IC83/ LY2603618, LY2606368, GDC-0425, PF-00477736, XL844, CEP-3891, SAR-020106, CCT-244747, Arry-575 (Thompson and Eastman, Br J Clin Pharmacol., 76(3): 358-369 (2013)), and SB218075. Exemplary ATR pathway inhibitors include, but are not limited to Schisandrin B, NU6027, NVP-BEZ235, VE-821, VE-822 (VX-970), AZ20, AZD6738, MIRIN, KU5593, VE-821, NU7441, LCA, and L189 (Weber and Ryan, Pharmacology & Therapeutics, 149:124-138 (2015)).

In some embodiments, the potentiating factor is a DNA polymerase alpha inhibitor, such as aphidicolin.

In some embodiments, the potentiating factor is a heat shock protein 90 inhibitor (HSP90i) such as STA-9090 (ganetespib). Other HSP90 inhibitors are known in the art and include, but are not limited to, benzoquinone ansamycin antibiotics such as geldanamycin (GA); 17-AAG (17-Allylamino-17-demethoxy-geldanamycin); 17-DMAG (17-dimethylaminoethylamino-17-demethoxy-geldanamycin) (Alvespimycin); IPI-504 (Retaspimycin); and AUY922 (Tatokoro, et al., EXCLI J., 14:48-58 (2015)).

E. Particle Delivery Vehicles

The compositions can include a biodegradable or bioerodible material in which the triplex-forming molecule is embedded or encapsulated.

The particles can be capable of controlled release of the active agent. The particles can be microparticle(s) and/or nanoparticle(s). The particles can include one or more polymers. One or more of the polymers can be a synthetic polymer. The particle or particles can be formed by, for example, single emulsion technique or double emulsion technique or nanoprecipitation.

In some embodiments, some of the compositions are packaged in particles and some are not. For example, a triplex-forming molecule and/or donor oligonucleotide can be incorporated into particles while a co-administered potentiating factor is not. In some embodiments, a triplex-forming molecule and/or donor oligonucleotide and a potentiating factor are both packaged in particles. Different compositions can be packaged in the same particles or different particles. For example, two or more active agents can be mixed and packaged together. In some embodiments, the different compositions are packaged separately into separate particles wherein the particles are similarly or identically composed and/or manufactured. In some embodiments, the different compositions are packaged separately into separate particles wherein the particles are differentially composed and/or manufactured.

The delivery vehicles can be nanoscale compositions, for example, 0.5 nm up to, but not including, about 1 micron. In some embodiments, and for some uses, the particles can be smaller, or larger. Thus, the particles can be microparticles, supraparticles, etc. For example, particle compositions can be between about 1 micron to about 1000 microns. Such compositions can be referred to as microparticulate compositions.

Nanoparticles generally refers to particles in the range of less than 0.5 nm up to, but not including 1,000 nm. In some embodiments, the nanoparticles have a diameter between 500 nm to less than 0.5 nm, or between 50 and 500 nm, or between 50 and 300 nm. Cellular internalization of polymeric particles can highly dependent upon their size, with nanoparticulate polymeric particles being internalized by cells with much higher efficiency than micoparticulate polymeric particles. For example, Desai, et al. have demonstrated that about 2.5 times more nanoparticles that are 100 nm in diameter are taken up by cultured Caco-2 cells as compared to microparticles having a diameter on 1 μM (Desai, et al., Pharm. Res., 14:1568-73 (1997)). Nanoparticles also have a greater ability to diffuse deeper into tissues in vivo.

The particles can have a mean particle size. Mean particle size generally refers to the statistical mean particle size (diameter) of the particles in the composition. Two populations can be said to have a substantially equivalent mean particle size when the statistical mean particle size of the first population of particles is within 20% of the statistical mean particle size of the second population of particles; more preferably within 15%, most preferably within 10%.

The weight average molecular weight can vary for a given polymer but is generally from about 1000 Daltons to 1,000,000 Daltons, 1000 Daltons to 500,000 Dalton, 1000 Daltons to 250,000 Daltons, 1000 Daltons to 100,000 Daltons, 5,000 Daltons to 100,000 Daltons, 5,000 Daltons to 75,000 Daltons, 5,000 Daltons to 50,000 Daltons, or 5,000 Daltons to 25,000 Daltons.

Particles are can be formed of one or more polymers. Exemplary polymers are discussed below. Copolymers such as random, block, or graft copolymers, or blends of the polymers listed below can also be used.

Functional groups on the polymer can be capped to alter the properties of the polymer and/or modify (e.g., decrease or increase) the reactivity of the functional group. For example, the carboxyl termini of carboxylic acid contain polymers, such as lactide- and glycolide-containing polymers, may optionally be capped, e.g., by esterification, and the hydroxyl termini may optionally be capped, e.g. by etherification or esterification.

Copolymers of PEG or derivatives thereof with any of the polymers described below may be used to make the polymeric particles. In certain embodiments, the PEG or derivatives may be located in the interior positions of the copolymer. Alternatively, the PEG or derivatives may locate near or at the terminal positions of the copolymer. For example, one or more of the polymers above can be terminated with a block of polyethylene glycol. In some embodiments, the core polymer is a blend of pegylated polymer and non-pegylated polymer, wherein the base polymer is the same (e.g., PLGA and PLGA-PEG) or different (e.g., PLGA-PEG and PLA). In certain embodiments, the microparticles or nanoparticles are formed under conditions that allow regions of PEG to phase separate or otherwise locate to the surface of the particles. The surface-localized PEG regions alone may perform the function of, or include, the surface-altering agent. In particular embodiments, the particles are prepared from one or more polymers terminated with blocks of polyethylene glycol as the surface-altering material.

In some embodiments, the particles may be used as nucleic acid carriers. In these embodiments, the particles can be formed of one or more cationic polymers which complex with one or more negatively charged nucleic acids.

The cationic polymer can be any synthetic or natural polymer bearing at least two positive charges per molecule and having sufficient charge density and molecular size to bind to nucleic acid under physiological conditions (i.e., pH and salt conditions encountered within the body or within cells). In certain embodiments, the polycationic polymer contains one or more amine residues.

Suitable cationic polymers include, for example, polyethylene imine (PEI), polyallylamine, polyvinylamine, polyvinylpyridine, aminoacetalized poly(vinyl alcohol), acrylic or methacrylic polymers (for example, poly(N,N-dimethylaminoethylmethacrylate)) bearing one or more amine residues, polyamino acids such as polyornithine, polyarginine, and polylysine, protamine, cationic polysaccharides such as chitosan, DEAE-cellulose, and DEAE-dextran, and polyamidoamine dendrimers (cationic dendrimer), as well as copolymers and blends thereof. In some embodiments, the polycationic polymer is poly(amine-co-ester), poly(amine-co-amide) polymer, or poly(amine-co-ester-co-ortho ester).

Cationic polymers can be either linear or branched, can be either homopolymers or copolymers, and when containing amino acids can have either L or D configuration, and can have any mixture of these features. Preferably, the cationic polymer molecule is sufficiently flexible to allow it to form a compact complex with one or more nucleic acid molecules.

In some embodiments, the cationic polymer has a molecular weight of between about 5,000 Daltons and about 100,000 Daltons, more preferably between about 5,000 and about 50,000 Daltons, most preferably between about 10,000 and about 35,000 Daltons.

In particular embodiments, the particles include a hydrophobic polymer, poly(amine-co-ester), poly(amine-co-amide) polymer, or poly(amine-co-ester-co-ortho ester), and optionally, but a shell of, for example, PEG. The core-shell particles can be formed by a co-block polymer. Exemplary polymers are provided below.

1. Exemplary Hydrophobic Polymers

The polymer that forms the core of the particle may be any biodegradable or non-biodegradable synthetic or natural polymer. In a preferred embodiment, the polymer is a biodegradable polymer.

Particles are ideal materials for the fabrication of gene editing delivery vehicles: 1) control over the size range of fabrication, down to 100 nm or less, an important feature for passing through biological barriers; 2) reproducible biodegradability without the addition of enzymes or cofactors; 3) capability for sustained release of encapsulated, protected nucleic acids over a period in the range of days to months by varying factors such as the monomer ratios or polymer size, for example, the ratio of lactide to glycolide monomer units in poly(lactide-co-glycolide) (PLGA); 4) well-understood fabrication methodologies that offer flexibility over the range of parameters that can be used for fabrication, including choices of the polymer material, solvent, stabilizer, and scale of production; and 5) control over surface properties facilitating the introduction of modular functionalities into the surface.

Any number of biocompatible polymers can be used to prepare the particles. In one embodiment, the biocompatible polymer(s) is biodegradable. In another embodiment, the particles are non-degradable. In other embodiments, the particles are a mixture of degradable and non-degradable particles.

Examples of preferred biodegradable polymers include synthetic polymers that degrade by hydrolysis such as poly(hydroxy acids), such as polymers and copolymers of lactic acid and glycolic acid, other degradable polyesters, polyanhydrides, poly(ortho)esters, polyesters, polyurethanes, poly(butyric acid), poly(valeric acid), poly(caprolactone), poly(hydroxyalkanoates), poly(lactide-co-caprolactone), and poly(amine-co-ester) polymers, such as those described in Zhou, et al., Nature Materials, 11(1):82-90 (2011), Tietjen, et al. Nature Communications, 8:191 (2017) doi:10.1038/s41467-017-00297-x, and WO 2013/082529, U.S. Published Application No. 2014/0342003, and PCT/US2015/061375.

Preferred natural polymers include alginate and other polysaccharides, collagen, albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Exemplary polymers include, but are not limited to, cyclodextrin-containing polymers, in particular cationic cyclodextrin-containing polymers, such as those described in U.S. Pat. No. 6,509,323,

In some embodiments, non-biodegradable polymers can be used, especially hydrophobic polymers. Examples of preferred non-biodegradable polymers include ethylene vinyl acetate, poly(meth)acrylic acid, copolymers of maleic anhydride with other unsaturated polymerizable monomers, poly(butadiene maleic anhydride), polyamides, copolymers and mixtures thereof, and dextran, cellulose and derivatives thereof.

Other suitable biodegradable and non-biodegradable polymers include, but are not limited to, polyanhydrides, polyamides, polycarbonates, polyalkylenes, polyalkylenes such as polyethylene and polypropylene, polyalkylene glycols such as poly(ethylene glycol) (PEG), polyalkylene oxides (PEO), polyalkylene terephthalates such as poly(ethylene terephthalate) and ethylene vinyl acetate polymer (EVA), polyvinyl alcohols, polyvinyl ethers, polyvinyl esters such as poly(vinyl acetate), polyethylene, polypropylene, poly(vinyl acetate), poly vinyl chloride, polystyrene, polyvinyl halides such as poly(vinyl chloride) (PVC), polyvinylpyrrolidone, polysiloxanes, polyvinylpyrrolidone, polymers of acrylic and methacrylic esters, polysiloxanes, polyurethanes and copolymers thereof, modified celluloses, alkyl cellulose, hydroxyalkyl celluloses, cellulose ethers, cellulose esters, nitro celluloses, hydroxypropylcellulose, cellulose acetate, cellulose propionate, cellulose acetate butyrate, cellulose acetate phthalate, carboxyethyl cellulose, cellulose triacetate, cellulose sulfate sodium salt, and polyacrylates such as poly(methyl methacrylate), poly(ethylmethacrylate), poly(2-hydroxyethyl methacrylate) (pHEMA), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate). These materials may be used alone, as physical mixtures (blends), or as co-polymers.

The polymer may be a bioadhesive polymer that is hydrophilic or hydrophobic. Hydrophilic polymers include CARBOPOL™ (a high molecular weight, crosslinked, acrylic acid-based polymers such as those manufactured by NOVEON™), polycarbophil, cellulose esters, and dextran. polymers of acrylic acids, include, but are not limited to, poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), and poly(octadecyl acrylate) (jointly referred to herein as “polyacrylic acids”).

Release rate controlling polymers may be included in the polymer matrix or in the coating on the formulation. Examples of rate controlling polymers that may be used are hydroxypropylmethylcellulose (HPMC) with viscosities of either 5, 50, 100 or 4000 cps or blends of the different viscosities, ethylcellulose, methylmethacrylates, such as EUDRAGIT® RS100, EUDRAGIT® RL100, EUDRAGIT® NE 30D (supplied by Rohm America). Gastrosoluble polymers, such as EUDRAGIT® E100 or enteric polymers such as EUDRAGIT® L100-55D, L100 and S100 may be blended with rate controlling polymers to achieve pH dependent release kinetics. Other hydrophilic polymers such as alginate, polyethylene oxide, carboxymethylcellulose, and hydroxyethylcellulose may be used as rate controlling polymers.

These polymers can be obtained from sources such as Sigma Chemical Co., St. Louis, Mo.; Polysciences, Warrenton, Pa.; Aldrich, Milwaukee, Wis.; Fluka, Ronkonkoma, N.Y.; and BioRad, Richmond, Calif., or can be synthesized from monomers obtained from these or other suppliers using standard techniques.

In certain embodiments, the hydrophobic polymer is an aliphatic polyester. In preferred embodiments, the hydrophobic polymer is polyhydroxyester such as poly(lactic acid), poly(glycolic acid), or poly(lactic acid-co-glycolic acid).

Other polymers include, but are not limited to, polyalkyl cyanoacralate, polyamino acids such as poly-L-lysine (PLL), poly(valeric acid), and poly-L-glutamic acid, hydroxypropyl methacrylate (HPMA), polyorthoesters, poly(ester amides), poly(ester ethers), polydioxanone and its copolymers, polyhydroxyalkanoates, polypropylene fumarate, polyoxymethylene, poly(butyric acid), trimethylene carbonate, and polyphosphazenes.

The particles can be designed to release molecules to be encapsulated or attached over a period of days to weeks. Factors that affect the duration of release include pH of the surrounding medium (higher rate of release at pH 5 and below due to acid catalyzed hydrolysis of PLGA) and polymer composition. Aliphatic polyesters differ in hydrophobicity and that in turn affects the degradation rate. The hydrophobic poly (lactic acid) (PLA), more hydrophilic poly (glycolic acid) PGA and their copolymers, poly (lactide-co-glycolide) (PLGA) may have different release rates. The degradation rate of these polymers, and often the corresponding drug release rate, can vary from days (PGA) to months (PLA) and is easily manipulated by varying the ratio of PLA to PGA.

In some preferred embodiments, the particles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide, collectively referred to herein as “PLA”, and caprolactone units, such as poly(8-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker. For example, particles can also contain one or more polymer conjugates containing end-to-end linkages between the polymer and a targeting moiety or a detectable label. For example, a modified polymer can be a PLGA-PEG-peptide block polymer.

The in vivo stability/release of the particles can be adjusted during the production by using polymers such as poly(lactide-co-glycolide) copolymerized with polyethylene glycol (PEG). If PEG is exposed on the external surface, it may increase the time these materials circulate due to the hydrophilicity of PEG.

A shell can also be formed of or contain a hyperbranched polymer (HP) with hydroxyl groups, such as a hyperbranched polyglycerol (HPG), hyperbranched peptides (HPP), hyperbranched oligonucleotides (HON), hyperbranched polysaccharides (HPS), and hyperbranched polyunsaturated or saturated fatty acids (HPF). The HP can be covalently bound to the one or more materials that form the core such that the hydrophilic HP is oriented towards the outside of the particles and the hydrophobic material oriented to form the core.

The HP coating can be modified to adjust the properties of the particles. For example, unmodified HP coatings impart stealth properties to the particles which resist non-specific protein absorption and are referred to as nonbioadhesive nanoparticles (NNPs). Alternatively, the hydroxyl groups on the HP coating can be chemically modified to form functional groups that react with functional groups on tissue or otherwise interact with tissue to adhere the particles to the tissue, cells, or extracellular materials, such as proteins. Such functional groups include, but are not limited to, aldehydes, amines, and O-substituted oximes. Particles with an HP coating chemically modified to form functional groups are referred to as bioadhesive nanoparticles (BNPs). The chemically modified HP coating of BNPs forms a bioadhesive corona of the particle surrounding the hydrophobic material forming the core. See, for example, WO 2015/172149, WO 2015/172153, WO 2016/183209, and U.S. Published Applications 2017/0000737 and 2017/0266119.

Particles can be formed of polymers fabricated from polylactides (PLA) and copolymers of lactide and glycolide (PLGA). These have established commercial use in humans and have a long safety record (Jiang, et al., Adv. Drug Deliv. Rev., 57(3):391-410); Aguado and Lambert, Immunobiology, 184(2-3):113-25 (1992); Bramwell, et al., Adv. Drug Deliv. Rev., 57(9):1247-65 (2005)). These polymers have been used to encapsulate siRNA (Yuan, et al., Jour. Nanosocience and Nanotechnology, 6:2821-8 (2006); Braden, et al., Jour. Biomed. Nanotechnology, 3:148-59 (2007); Khan, et al., Jour. Drug Target, 12:393-404 (2004); Woodrow, et al., Nature Materials, 8:526-533 (2009)). Murata, et al., J. Control. Release, 126(3):246-54 (2008) showed inhibition of tumor growth after intratumoral injection of PLGA microspheres encapsulating siRNA targeted against vascular endothelial growth factor (VEGF). However, these microspheres were too large to be endocytosed (35-45 μm) (Conner and Schmid, Nature, 422(6927):37-44 (2003)) and required release of the anti-VEGF siRNA extracellularly as a polyplex with either polyarginine or PEI before they could be internalized by the cell. These microparticles may have limited applications because of the toxicity of the polycations and the size of the particles. Nanoparticles (100-300 nm) of PLGA can penetrate deep into tissue and are easily internalized by many cells (Conner and Schmid, Nature, 422(6927):37-44 (2003)).

Exemplary particles are described in U.S. Pat. Nos. 4,883,666, 5,114,719, 5,601,835, 7,534,448, 7,534,449, 7,550,154, and 8,889,117, and U.S. Published Application Nos. 2009/0269397, 2009/0239789, 2010/0151436, 2011/0008451, 2011/0268810, 2014/0342003, 2015/0118311, 2015/0125384, 2015/0073041, Hubbell, et al., Science, 337:303-305 (2012), Cheng, et al., Biomaterials, 32:6194-6203 (2011), Rodriguez, et al., Science, 339:971-975 (2013), Hrkach, et al., Sci Transl Med., 4:128ra139 (2012), McNeer, et al., Mol Ther., 19:172-180 (2011), McNeer, et al., Gene Ther., 20:658-659 (2013), Babar, et al., Proc Natl Acad Sci USA, 109:E1695-E1704 (2012), Fields, et al., J Control Release 164:41-48 (2012), and Fields, et al., Advanced Healthcare Materials, 361-366 (2015).

2. Poly(amine-co-esters), Poly(amine-co-amides), and Poly(amine-co-ester-co-ortho esters)

The core of the particles can be formed of or contain one or more poly(amine-co-ester), poly(amine-co-amide), poly(amine-co-ester-co-ortho ester) or a combination thereof. In some embodiments, the particles are polyplexes. In some embodiments, the content of a hydrophobic monomer in the polymer is increased relative the content of the same hydrophobic monomer when used to form polyplexes. Increasing the content of a hydrophobic monomer in the polymer forms a polymer that can form solid core particles in the presence of nucleic acids. Unlike polyplexes, these particles are stable for long periods of time during incubation in buffered water, or serum, or upon administration (e.g., injection) into animals. They also provide for a sustained release of nucleic acids which leads to long term activity. In some aspects, the molecular weight of the polymer is less than 5 kDa, 7.5 kDa, 10 kDa, 20 kDa, or 25 kDa. In some forms the molecular weight of the polymer is between about 1 kDa and about 25 kDa, between about 1 kDa and about 10 kDa, between about 1 kDa and about 7.5 kDa.

The polymers can have the general formula:

((A)ₓ-(B)_{y}-(C)_{q}-(D)_{w}-(E)_{f})ₕ,

wherein A, B, C, D, and E independently include monomeric units derived from lactones (such as pentadecalactone), a polyfunctional molecule (such as N-methyldiethanolamine), a diacid or diester (such as diethylsebacate), an ortho ester, or polyalkylene oxide (such as polyethylene glycol). In some aspects, the polymers include at least a lactone, a polyfunctional molecule, and a diacid or diester monomeric units. In some aspects, the polymers include at least a lactone, a polyfunctional molecule, an ortho ester, and a diacid or diester monomeric units. In general, the polyfunctional molecule contains one or more cations, one or more positively ionizable atoms, or combinations thereof. The one or more cations are formed from the protonation of a basic nitrogen atom, or from quaternary nitrogen atoms.

In general, x, y, q, w, and f are independently integers from 0-1000, with the proviso that the sum (x+y+q+w+f) is greater than one. h is an integer from 1 to 1000.

In some forms, the percent composition of the lactone can be between about 30% and about 100%, calculated as the mole percentage of lactone unit vs. (lactone unit+diester/diacid). Expressed in terms of molar ratio, the lactone unit vs. (lactone unit+diester/diacid) content is between about 0.3 and about 1. Preferably, the number of carbon atoms in the lactone unit is between about 10 and about 24. In some embodiments, the number of carbon atoms in the lactone unit is between about 12 and about 16. In some embodiments, the number of carbon atoms in the lactone unit is 12 (dodecalactone), 15 (pentadecalactone), or 16 (hexadecalactone).

The molecular weight of the lactone unit in the polymer, the lactone unit's content of the polymer, or both, influences the formation of solid core particles.

Suitable polymers as well as particles and polyplexes formed therefrom are disclosed in WO 2013/082529, WO 2016/183217, U.S. Published Application No. 2016/0251477, U.S. Published Application No. 2015/0073041, U.S. Published Application No. 2014/0073041, and U.S. Pat. No. 9,272,043, each of which is specifically incorporated by reference in entirety.

F. Polycations

In some embodiments, the nucleic acids are complexed to polycations to increase the encapsulation efficiency of the nucleic acids into the particles. The term “polycation” refers to a compound having a positive charge, preferably at least 2 positive charges, at a selected pH, preferably physiological pH. Polycationic moieties have between about 2 to about 15 positive charges, preferably between about 2 to about 12 positive charges, and more preferably between about 2 to about 8 positive charges at selected pH values.

Many polycations are known in the art. Suitable constituents of polycations include basic amino acids and their derivatives such as arginine, asparagine, glutamine, lysine and histidine; cationic dendrimers; and amino polysaccharides. Suitable polycations can be linear, such as linear tetralysine, branched or dendrimeric in structure.

Exemplary polycations include, but are not limited to, synthetic polycations based on acrylamide and 2-acrylamido-2-methylpropanetrimethylamine, poly(N-ethyl-4-vinylpyridine) or similar quartemized polypyridine, diethylaminoethyl polymers and dextran conjugates, polymyxin B sulfate, lipopolyamines, poly(allylamines) such as the strong polycation poly(dimethyldiallylammonium chloride), polyethyleneimine, polybrene, and polypeptides such as protamine, the histone polypeptides, polylysine, polyarginine and polyornithine.

In one embodiment, the polycation is a polyamine Polyamines are compounds having two or more primary amine groups. In a preferred embodiment, the polyamine is a naturally occurring polyamine that is produced in prokaryotic or eukaryotic cells. Naturally occurring polyamines represent compounds with cations that are found at regularly-spaced intervals and are therefore particularly suitable for complexing with nucleic acids. Polyamines play a major role in very basic genetic processes such as DNA synthesis and gene expression. Polyamines are integral to cell migration, proliferation and differentiation in plants and animals. The metabolic levels of polyamines and amino acid precursors are critical and hence biosynthesis and degradation are tightly regulated. Suitable naturally occurring polyamines include, but are not limited to, spermine, spermidine, cadaverine and putrescine. In a preferred embodiment, the polyamine is spermidine.

In another embodiment, the polycation is a cyclic polyamine Cyclic polyamines are known in the art and are described, for example, in U.S. Pat. No. 5,698,546, WO 1993/012096 and WO 2002/010142. Exemplary cyclic polyamines include, but are not limited to, cyclen.

Spermine and spermidine are derivatives of putrescine (1,4-diaminobutane), which is produced from L-ornithine by action of ODC (ornithine decarboxylase). L-ornithine is the product of L-arginine degradation by arginase. Spermidine is a triamine structure that is produced by spermidine synthase (SpdS) which catalyzes monoalkylation of putrescine (1,4-diaminobutane) with decarboxylated S-adenosylmethionine (dcAdoMet) 3-aminopropyl donor. The formal alkylation of both amino groups of putrescine with the 3-aminopropyl donor yields the symmetrical tetraamine spermine. The biosynthesis of spermine proceeds to spermidine by the effect of spermine synthase (SpmS) in the presence of dcAdoMet. The 3-aminopropyl donor (dcAdoMet) is derived from S-adenosylmethionine by sequential transformation of L-methionine by methionine adenosyltransferase followed by decarboxylation by AdoMetDC (S-adenosylmethionine decarboxylase). Hence, putrescine, spermidine and spermine are metabolites derived from the amino acids L-arginine (L-ornithine, putrescine) and L-methionine (dcAdoMet, aminopropyl donor).

In some embodiments, the particles themselves are a polycation (e.g., a blend of PLGA and poly(beta amino ester).

G. Functional Molecules

Functional molecules can be associated with, linked, conjugated, or otherwise attached directly or indirectly triplex-forming molecules, potentiating agents, or particles utilized for delivery thereof. For example, the composition can include a targeting agent, a cell penetrating peptide, or a combination thereof. In some embodiments, two or more targeting molecules are used. Target agents can be bound or conjugated to particles (e.g., a polymer of the particle).

1. Targeting Molecules

One class of functional elements is targeting molecules. Targeting molecules can be associated with, linked, conjugated, or otherwise attached directly or indirectly to the gene editing molecule, or to a particle or other delivery vehicle thereof.

Targeting molecules can be proteins, peptides, nucleic acid molecules, saccharides or polysaccharides that bind to a receptor or other molecule on the surface of a targeted cell. The degree of specificity and the avidity of binding to the target cells can be modulated through the selection of the targeting molecule. For example, antibodies are very specific. These can be polyclonal, monoclonal, fragments, recombinant, or single chain, many of which are commercially available or readily obtained using standard techniques.

Examples of moieties include, for example, targeting moieties which provide for the delivery of molecules to specific cells, e.g., antibodies to hematopoietic stem cells, CD34+ cells, epithelial cells, T cells or any other preferred cell type, as well as receptor and ligands expressed on the preferred cell type. In some embodiments, the moieties target hematopoietic stem cells.

In some embodiments, the targeting molecule targets a cell surface protein.

The choice of targeting molecule will depend on the method of administration of the particle composition and the cells or tissues to be targeted. The targeting molecule may generally increase the binding affinity of the particles for cell or tissues or may target the particle to a particular tissue in an organ or a particular cell type in a tissue.

2. Protein Transduction Domains and Fusogenic Peptides

Other functional elements that can be associated with, linked, conjugated, or otherwise attached directly or indirectly to the triplex-forming molecule, potentiating agent, or to a particle or other delivery vehicle thereof, include protein transduction domains and fusogenic peptides.

For example, the efficiency of particle delivery systems can also be improved by the attachment of functional ligands to the particle surface. Potential ligands include, but are not limited to, small molecules, cell-penetrating peptides (CPPs), targeting peptides, antibodies or aptamers (Yu, et al., PLoS One., 6:e24077 (2011), Cu, et al., J Control Release, 156:258-264 (2011), Nie, et al., J Control Release, 138:64-70 (2009), Cruz, et al., J Control Release, 144:118-126 (2010)). Attachment of these moieties serves a variety of different functions; such as inducing intracellular uptake, endosome disruption, and delivery of the plasmid payload to the nucleus.

There have been numerous methods employed to tether ligands to the particle surface. One approach is direct covalent attachment to the functional groups on PLGA NPs (Bertram, Acta Biomater. 5:2860-2871 (2009)). Another approach utilizes amphiphilic conjugates like avidin palmitate to secure biotinylated ligands to the NP surface (Fahmy, et al., Biomaterials, 26:5727-5736 (2005), Cu, et al., Nanomedicine, 6:334-343 (2010)). This approach produces particles with enhanced uptake into cells, but reduced pDNA release and gene transfection, which is likely due to the surface modification occluding pDNA release. In a similar approach, lipid-conjugated polyethylene glycol (PEG) is used as a multivalent linker of penetratin, a CPP, or folate (Cheng, et al., Biomaterials, 32:6194-6203 (2011)).

These methods, as well as other methods discussed herein, and others methods known in the art, can be combined to tune particle function and efficacy. In some preferred embodiments, PEG is used as a linker for linking functional molecules to particles. For example, DSPE-PEG(2000)-maleimide is commercially available and can be used utilized for covalently attaching functional molecules such as CPP.

“Protein Transduction Domain” or PTD refers to a polypeptide, polynucleotide, or organic or inorganic compounds that facilitates traversing a lipid bilayer, micelle, cell membrane, organelle membrane, or vesicle membrane. A PTD attached to another molecule facilitates the molecule traversing membranes, for example going from extracellular space to intracellular space, or cytosol to within an organelle. PTA can be short basic peptide sequences such as those present in many cellular and viral proteins. Exemplary protein transduction domains that are well-known in the art include, but are not limited to, the Antennapedia PTD and the TAT (transactivator of transcription) PTD, poly-arginine, poly-lysine or mixtures of arginine and lysine, HIV TAT (YGRKKRRQRRR (SEQ ID NO:1) or RKKRRQRRR (SEQ ID NO:2), 11 arginine residues, VP22 peptide, and an ANTp peptide (RQIKIWFQNRRMKWKK) (SEQ ID NO:3) or positively charged polypeptides or polynucleotides having 8-15 residues, preferably 9-11 residues. Short, non-peptide polymers that are rich in amines or guanidinium groups are also capable of carrying molecules crossing biological membranes. Penetratin and other derivatives of peptides derived from antennapedia (Cheng, et al., Biomaterials, 32(26):6194-203 (2011) can also be used. Results show that penetratin in which additional Args are added, further enhances uptake and endosomal escape, and IKK NBD, which has an antennapedia domain for permeation as well as a domain that blocks activation of NFkB and has been used safely in the lung for other purposes (von Bismarck, et al., Pulmonary Pharmacology & Therapeutics, 25(3):228-35 (2012), Kamei, et al., Journal Of Pharmaceutical Sciences, 102(11):3998-4008 (2013)).

A “fusogenic peptide” is any peptide with membrane destabilizing abilities. In general, fusogenic peptides have the propensity to form an amphiphilic alpha-helical structure when in the presence of a hydrophobic surface such as a membrane. The presence of a fusogenic peptide induces formation of pores in the cell membrane by disruption of the ordered packing of the membrane phospholipids. Some fusogenic peptides act to promote lipid disorder and in this way enhance the chance of merging or fusing of proximally positioned membranes of two membrane enveloped particles of various nature (e.g. cells, enveloped viruses, liposomes). Other fusogenic peptides may simultaneously attach to two membranes, causing merging of the membranes and promoting their fusion into one. Examples of fusogenic peptides include a fusion peptide from a viral envelope protein ectodomain, a membrane-destabilizing peptide of a viral envelope protein membrane-proximal domain from the cytoplasmic tails.

Other fusogenic peptides often also contain an amphiphilic-region. Examples of amphiphilic-region containing peptides include: melittin, magainins, the cytoplasmic tail of HIV1 gp41, microbial and reptilian cytotoxic peptides such as bomolitin 1, pardaxin, mastoparan, crabrolin, cecropin, entamoeba, and staphylococcal .alpha.-toxin; viral fusion peptides from (1) regions at the N terminus of the transmembrane (TM) domains of viral envelope proteins, e.g. HIV-1, SIV, influenza, polio, rhinovirus, and coxsackie virus; (2) regions internal to the TM ectodomain, e.g. semliki forest virus, sindbis virus, rota virus, rubella virus and the fusion peptide from sperm protein PH-30: (3) regions membrane-proximal to the cytoplasmic side of viral envelope proteins e.g. in viruses of avian leukosis (ALV), Feline immunodeficiency (FIV), Rous Sarcoma (RSV), Moloney murine leukemia virus (MoMuLV), and spleen necrosis (SNV).

In particular embodiments, a functional molecule such as a CPP is covalently linked to DSPE-PEG-maleimide functionalized particles such as PBAE/PLGA blended particles using known methods such as those described in Fields, et al., J Control Release, 164(1):41-48 (2012). For example, DSPE-PEG-function molecule can be added to the 5.0% PVA solution during formation of the second emulsion. In some embodiments, the loading ratio is about 5 nmol/mg ligand-to-polymer ratio.

In some embodiments, the functional molecule is a CPP such as those above, or mTAT (HIV-1 (with histidine modification) HHHHRKKRRQRRRRHHHHH (SEQ ID NO:4) (Yamano, et al., J Control Release, 152:278-285 (2011)); or bPrPp (Bovine prion) MVKSKIGSWILVLFVAMWS DVGLCKKRPKP (SEQ ID NO:5) (Magzoub, et al., Biochem Biophys Res Commun., 348:379-385 (2006)); or MPG (Synthetic chimera: SV40 Lg T. Ant.+HIV gb41 coat) GALFLGFLGAAGSTMGAWSQPKKKRKV (SEQ ID NO:6) (Endoh, et al., Adv Drug Deliv Rev., 61:704-709 (2009)).

### III. Methods of Use

A. Methods of Treatment

The disclosed compositions can be used for ex vivo or in vivo gene editing. The methods typically include contacting a cell with an effective amount of triplex forming molecules, preferably in combination with a donor oligonucleotide, optionally in combination with a potentiating agent, to modify the cell's genome. As discussed in more detail below, the contacting can occur ex vivo or in vivo. In preferred embodiments, the method includes contacting a population of target cells with an effective amount of the composition, to modify the genomes of a sufficient number of cells to achieve a therapeutic result.

For example, the effective amount or therapeutically effective amount can be a dosage sufficient to treat, inhibit, or alleviate one or more symptoms of a disease or disorder, or to otherwise provide a desired pharmacologic and/or physiologic effect, for example, reducing, inhibiting, or reversing one or more of the underlying pathophysiological mechanisms underlying a disease or disorder.

The molecules can be administered in an effective amount to induce formation of a triple helix at the target site. An effective amount of triplex-forming molecules may also be an amount effective to increase the rate of recombination of a donor fragment relative to administration of the donor fragment in the absence of the gene editing technology.

The formulation is made to suit the mode of administration. Pharmaceutically acceptable carriers are determined in part by the particular composition being administered, as well as by the particular method used to administer the composition. Accordingly, there is a wide variety of suitable formulations of pharmaceutical compositions containing the nucleic acids. The precise dosage will vary according to a variety of factors such as subject-dependent variables (e.g., age, immune system health, clinical symptoms etc.). Exemplary symptoms, pharmacologic, and physiologic effects are discussed in more detail below.

The disclosed compositions can be administered to or otherwise contacted with target cells once, twice, or three time daily; one, two, three, four, five, six, seven times a week, one, two, three, four, five, six, seven or eight times a month. For example, in some embodiments, the composition is administered every two or three days, or on average about 2 to about 4 times about week.

In some embodiments, the potentiating agent is administered to the subject prior to administration of the gene editing technology to the subject.

The potentiating agent can be administered to the subject, for example, 1, 2, 3, 4, 5, 6, 8, 10, 12, 18, or 24 hours, or 1, 2, 3, 4, 5, 6, or 7 days, or any combination thereof prior to administration of the gene editing technology to the subject.

In some embodiments, the gene editing technology is administered to the subject prior to administration of the potentiating agent to the subject. The gene editing technology can be administered to the subject, for example, 1, 2, 3, 4, 5, 6, 8, 10, 12, 18, or 24 hours, or 1, 2, 3, 4, 5, 6, or 7 days, or any combination thereof prior to administration of the potentiating agent to the subject.

In preferred embodiments, the compositions are administered in an amount effective to induce gene modification in at least one target allele to occur at frequency of at least 0.1, 0.2. 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, or 25% of target cells. In some embodiments, particularly ex vivo applications, gene modification occurs in at least one target allele at a frequency of about 0.1-25%, or 0.5-25%, or 1-25% 2-25%, or 3-25%, or 4-25% or 5-25% or 6-25%, or 7-25%, or 8-25%, or 9-25%, or 10-25%, 11-25%, or 12-25%, or 13%-25% or 14%-25% or 15-25%, or 2-20%, or 3-20%, or 4-20% or 5-20% or 6-20%, or 7-20%, or 8-20%, or 9-20%, or 10-20%, 11-20%, or 12-20%, or 13%-20% or 14%-20% or 15-20%, 2-15%, or 3-15%, or 4-15% or 5-15% or 6-15%, or 7-15%, or 8-15%, or 9-15%, or 10-15%, 11-15%, or 12-15%, or 13%-15% or 14%-15%.

In some embodiments, particularly in vivo applications, gene modification occurs in at least one target allele at a frequency of about 0.1% to about 10%, or about 0.2% to about 10%, or about 0.3% to about 10%, or about 0.4% to about 10%, or about 0.5% to about 10%, or about 0.6% to about 10%, or about 0.7% to about 10%, or about 0.8% to about 10%, or about 0.9% to about 10%, or about 1.0% to about 10% , or about 1.1% to about 10%, or about 1.1% to about 10%, 1.2% to about 10%, or about 1.3% to about 10%, or about 1.4% to about 10%, or about 1.5% to about 10%, or about 1.6% to about 10%, or about 1.7% to about 10%, or about 1.8% to about 10%, or about 1.9% to about 10%, or about 2.0% to about 10%, or about 2.5% to about 10% , or about 3.0% to about 10%, or about 3.5% to about 10%, or about 4.0% to about 10%, or about 4.5% to about 10%, or about 5.0% to about 10%.

In some embodiments, gene modification occurs with low off-target effects. In some embodiments, off-target modification is undetectable using routine analysis such as those described in the Examples below. In some embodiments, off-target incidents occur at a frequency of 0-1%, or 0-0.1%, or 0-0.01%, or 0-0.001%, or 0-0.0001%, or 0-0000.1%, or 0-0.000001%. In some embodiments, off-target modification occurs at a frequency that is about 10², 10³, 10⁴, or 10⁵-fold lower than at the target site.

In general, by way of example only, dosage forms useful in the disclosed methods can include doses in the range of about 10² to about 10⁵⁰, or about 10⁵ to about 10⁴⁰, or about 10¹⁰ to about 10³⁰, or about 10¹² to about 10²⁰ copies of triplex-forming molecules and optionally donor oligonucleotide per dose. In particular embodiments, about 10¹³, 10¹⁴, 10¹⁵, 10¹⁶, or 10¹⁷ copies of triplex-forming molecules and optionally donor oligonucleotide are administered to a subject in need thereof.

In other embodiments, dosages are expressed in moles. For example, in some embodiments, the dose of triplex-forming molecules and optionally donor oligonucleotide is about 0.1 nmol to about 100 nmol, or about 0.25 nmol to about 50 nmol, or about 0.5 nmol to about 25 nmol, or about 0.75 nmol to about 7.5 nmol.

In other embodiments, dosages are expressed in molecules per target cell. For example, in some embodiments, the dose of triplex-forming molecules and optionally donor oligonucleotide is about 10² to about 10⁵⁰, or about 10⁵ to about 10¹⁵, or about 10⁷ to about 10¹², or about 10⁸ to about 10¹¹ copies of the triplex-forming molecules and optionally donor oligonucleotide per target cell.

In other embodiments, dosages are expressed in mg/kg, particularly when the expressed as an in vivo dosage of triplex-forming molecules and optionally donor oligonucleotide packaged in a nanoparticle with or without functional molecules. Dosages can be, for example 0.1 mg/kg to about 1,000 mg/kg, or 0.5 mg/kg to about 1,000 mg/kg, or 1 mg/kg to about 1,000 mg/kg, or about 10 mg/kg to about 500 mg/kg, or about 20 mg/kg to about 500 mg/kg per dose, or 20 mg/kg to about 100 mg/kg per dose, or 25 mg/kg to about 75 mg/kg per dose, or about 25, 30, 35, 40, 45, 50, 55, 60, 65, 70, or 75 mg/kg per dose.

In other embodiments, dosages are expressed in mg/ml, particularly when the expressed as an ex vivo dosage of triplex-forming molecules and optionally donor oligonucleotide packaged in a nanoparticle with or without functional molecules. Dosages can be, for example 0.01 mg/ml to about 100 mg/ml, or about 0.5 mg/ml to about 50 mg/ml, or about 1 mg/ml to about 10 mg/ml per dose to a cell population of 10⁶ cells.

As discussed above, triplex-forming molecules can be administered without, but is preferably administered with at least one donor oligonucleotide. Such donors can be administered at similar dosages as the triplex-forming molecules. Compositions should include an amount of donor fragment effective to recombine at the target site in the presence of a triplex forming molecule.

Potentiating Agents

The methods can include contacting cells with an effective amount potentiating agents. Preferably the amount of potentiating agent is effective to increase gene modification when used in combination with a triplex-forming molecule and optionally donor oligonucleotide, compared to using the gene modifying technology in the absence of the potentiating agent.

Exemplary dosages for SCF include, about 0.01 mg/kg to about 250 mg/kg, or about 0.1 mg/kg to about 100 mg/kg, or about 0.5 mg/kg to about 50 mg/kg, or about 0.75 mg/kg to about 10 mg/kg.

Dosages for CHK1 inhibitors are known in the art, and many of these are in clinical trial. Accordingly, the dosage can be selected by the practitioner based on known, preferred human dosages. In preferred embodiments, the dosage is below the lowest-observed-adverse-effect level (LOAEL), and is preferably a no observed adverse effect level (NOAEL) dosage.

1. Ex Vivo Gene Therapy

In some embodiments, ex vivo gene therapy of cells is used for the treatment of a genetic disorder in a subject. For ex vivo gene therapy, cells are isolated from a subject and contacted ex vivo with the compositions to produce cells containing mutations in or adjacent to genes. In a preferred embodiment, the cells are isolated from the subject to be treated or from a syngeneic host. Target cells are removed from a subject prior to contacting with a gene editing composition and preferably a potentiating factor. The cells can be hematopoietic progenitor or stem cells. In a preferred embodiment, the target cells are CD34⁺ hematopoietic stem cells. Hematopoietic stem cells (HSCs), such as CD34+ cells are multipotent stem cells that give rise to all the blood cell types including erythrocytes. Therefore, CD34+ cells can be isolated from a patient with, for example, thalassemia, sickle cell disease, or a lysosomal storage disease, the mutant gene altered or repaired ex-vivo using the disclosed compositions and methods, and the cells reintroduced back into the patient as a treatment or a cure.

Stem cells can be isolated and enriched by one of skill in the art. Methods for such isolation and enrichment of CD34⁺ and other cells are known in the art and disclosed for example in U.S. Pat. Nos. 4,965,204; 4,714,680; 5,061,620; 5,643,741; 5,677,136; 5,716,827; 5,750,397 and 5,759,793. As used herein in the context of compositions enriched in hematopoietic progenitor and stem cells, “enriched” indicates a proportion of a desirable element (e.g. hematopoietic progenitor and stem cells) which is higher than that found in the natural source of the cells. A composition of cells may be enriched over a natural source of the cells by at least one order of magnitude, preferably two or three orders, and more preferably 10, 100, 200 or 1000 orders of magnitude.

In humans, CD34⁺ cells can be recovered from cord blood, bone marrow or from blood after cytokine mobilization effected by injecting the donor with hematopoietic growth factors such as granulocyte colony stimulating factor (G-CSF), granulocyte-monocyte colony stimulating factor (GM-CSF), stem cell factor (SCF) subcutaneously or intravenously in amounts sufficient to cause movement of hematopoietic stem cells from the bone marrow space into the peripheral circulation. Initially, bone marrow cells may be obtained from any suitable source of bone marrow, e.g. tibiae, femora, spine, and other bone cavities. For isolation of bone marrow, an appropriate solution may be used to flush the bone, which solution will be a balanced salt solution, conveniently supplemented with fetal calf serum or other naturally occurring factors, in conjunction with an acceptable buffer at low concentration, generally from about 5 to 25 mM. Convenient buffers include Hepes, phosphate buffers, lactate buffers, etc.

Cells can be selected by positive and negative selection techniques. Cells can be selected using commercially available antibodies which bind to hematopoietic progenitor or stem cell surface antigens, e.g. CD34, using methods known to those of skill in the art. For example, the antibodies may be conjugated to magnetic beads and immunogenic procedures utilized to recover the desired cell type. Other techniques involve the use of fluorescence activated cell sorting (FACS). The CD34 antigen, which is found on progenitor cells within the hematopoietic system of non-leukemic individuals, is expressed on a population of cells recognized by the monoclonal antibody My-10 (i.e., express the CD34 antigen) and can be used to isolate stem cell for bone marrow transplantation. My-10 deposited with the American Type Culture Collection (Rockville, Md.) as HB-8483 is commercially available as anti-HPCA 1. Additionally, negative selection of differentiated and “dedicated” cells from human bone marrow can be utilized, to select against substantially any desired cell marker. For example, progenitor or stem cells, most preferably CD34⁺ cells, can be characterized as being any of CD3⁻, CD7⁻, CD8⁻, CD10⁻, CD14⁻, CD15⁻, CD19⁻, CD20⁻, CD33⁻, Class II HLA⁺ and Thy-1⁺.

Once progenitor or stem cells have been isolated, they may be propagated by growing in any suitable medium. For example, progenitor or stem cells can be grown in conditioned medium from stromal cells, such as those that can be obtained from bone marrow or liver associated with the secretion of factors, or in medium including cell surface factors supporting the proliferation of stem cells. Stromal cells may be freed of hematopoietic cells employing appropriate monoclonal antibodies for removal of the undesired cells.

The isolated cells are contacted ex vivo with a combination of triplex-forming molecules and donor oligonucleotides in amounts effective to cause the desired mutations in or adjacent to genes in need of repair or alteration, for example the human beta-globin or α-L-iduronidase gene. These cells are referred to herein as modified cells. Methods for transfection of cells with oligonucleotides and peptide nucleic acids are well known in the art (Koppelhus, et al., Adv. Drug Deliv. Rev., 55(2): 267-280 (2003)). It may be desirable to synchronize the cells in S-phase to further increase the frequency of gene correction. Methods for synchronizing cultured cells, for example, by double thymidine block, are known in the art (Zielke, et al., Methods Cell Biol., 8:107-121 (1974)).

The modified cells can be maintained or expanded in culture prior to administration to a subject. Culture conditions are generally known in the art depending on the cell type. Conditions for the maintenance of CD34⁺ in particular have been well studied, and several suitable methods are available.

A common approach to ex vivo multi-potential hematopoietic cell expansion is to culture purified progenitor or stem cells in the presence of early-acting cytokines such as interleukin-3. It has also been shown that inclusion, in a nutritive medium for maintaining hematopoietic progenitor cells ex vivo, of a combination of thrombopoietin (TPO), stem cell factor (SCF), and flt3 ligand (Flt-3L; i.e., the ligand of the flt3 gene product) was useful for expanding primitive (i.e., relatively non-differentiated) human hematopoietic progenitor cells in vitro, and that those cells were capable of engraftment in SCID-hu mice (Luens et al., 1998, Blood 91:1206-1215). In other known methods, cells can be maintained ex vivo in a nutritive medium (e.g., for minutes, hours, or 3, 6, 9, 13, or more days) including murine prolactin-like protein E (mPLP-E) or murine prolactin-like protein F (mPIP-F; collectively mPLP-E/IF) (U.S. Pat. No. 6,261,841). It will be appreciated that other suitable cell culture and expansion method can be used in accordance with the invention as well. Cells can also be grown in serum-free medium, as described in U.S. Pat. No. 5,945,337.

In another embodiment, the modified hematopoietic stem cells are differentiated ex vivo into CD4⁺ cells culture using specific combinations of interleukins and growth factors prior to administration to a subject using methods well known in the art. The cells may be expanded ex vivo in large numbers, preferably at least a 5-fold, more preferably at least a 10-fold and even more preferably at least a 20-fold expansion of cells compared to the original population of isolated hematopoietic stem cells.

In another embodiment cells for ex vivo gene therapy, the cells to be used can be dedifferentiated somatic cells. Somatic cells can be reprogrammed to become pluripotent stem-like cells that can be induced to become hematopoietic progenitor cells. The hematopoietic progenitor cells can then be treated with triplex-forming molecules and donor oligonucleotides as described above with respect to CD34⁺ cells to produce recombinant cells having one or more modified genes. Representative somatic cells that can be reprogrammed include, but are not limited to fibroblasts, adipocytes, and muscles cells. Hematopoietic progenitor cells from induced stem-like cells have been successfully developed in the mouse (Hanna, J. et al. Science, 318:1920-1923 (2007)).

To produce hematopoietic progenitor cells from induced stem-like cells, somatic cells are harvested from a host. In a preferred embodiment, the somatic cells are autologous fibroblasts. The cells are cultured and transduced with vectors encoding Oct4, Sox2, Klf4, and c-Myc transcription factors. The transduced cells are cultured and screened for embryonic stem cell (ES) morphology and ES cell markers including, but not limited to AP, SSEA1, and Nanog. The transduced ES cells are cultured and induced to produce induced stem-like cells. Cells are then screened for CD41 and c-kit markers (early hematopoietic progenitor markers) as well as markers for myeloid and erythroid differentiation.

The modified hematopoietic stem cells or modified induced hematopoietic progenitor cells are then introduced into a subject. Delivery of the cells may be effected using various methods and includes most preferably intravenous administration by infusion as well as direct depot injection into periosteal, bone marrow and/or subcutaneous sites.

The subject receiving the modified cells may be treated for bone marrow conditioning to enhance engraftment of the cells. The recipient may be treated to enhance engraftment, using a radiation or chemotherapeutic treatment prior to the administration of the cells. Upon administration, the cells will generally require a period of time to engraft. Achieving significant engraftment of hematopoietic stem or progenitor cells typically takes weeks to months.

A high percentage of engraftment of modified hematopoietic stem cells is not envisioned to be necessary to achieve significant prophylactic or therapeutic effect. It is expected that the engrafted cells will expand over time following engraftment to increase the percentage of modified cells. It is expected that engraftment of only a small number or small percentage of modified hematopoietic stem cells will be required to provide a prophylactic or therapeutic effect.

In preferred embodiments, the cells to be administered to a subject will be autologous, e.g. derived from the subject, or syngenic.

In some embodiments, the compositions and methods can be used to edit embryonic genomes in vitro. The methods typically include contacting an embryo in vitro with an effective amount of potentiating agent and gene editing technology to induce at least one alteration in the genome of the embryo. Most preferably the embryo is a single cell zygote, however, treatment of male and female gametes prior to and during fertilization, and embryos having 2, 4, 8, or 16 cells and including not only zygotes, but also morulas and blastocytes, are also provided. Typically, the embryo is contacted with the compositions on culture days 0-6 during or following in vitro fertilization.

The contacting can be adding the compositions to liquid media bathing the embryo. For example, the compositions can be pipetted directly into the embryo culture media, whereupon they are taken up by the embryo.

2. In Vivo Gene Therapy

The disclosed compositions can be administered directly to a subject for in vivo gene therapy.

a. Pharmaceutical Formulations

The disclosed compositions are preferably employed for therapeutic uses in combination with a suitable pharmaceutical carrier. Such compositions include an effective amount of the composition, and a pharmaceutically acceptable carrier or excipient.

It is understood by one of ordinary skill in the art that nucleotides administered in vivo are taken up and distributed to cells and tissues (Huang, et al., FEBS Lett., 558(1-3):69-73 (2004)). For example, Nyce, et al. have shown that antisense oligodeoxynucleotides (ODNs) when inhaled bind to endogenous surfactant (a lipid produced by lung cells) and are taken up by lung cells without a need for additional carrier lipids (Nyce, et al., Nature, 385:721-725 (1997)). Small nucleic acids are readily taken up into T24 bladder carcinoma tissue culture cells (Ma, et al., Antisense Nucleic Acid Drug Dev., 8:415-426 (1998)).

The disclosed compositions including triplex-forming molecules, such as TFOs and PNAs, and donor fragments may be in a formulation for administration topically, locally or systemically in a suitable pharmaceutical carrier. Remington's Pharmaceutical Sciences, 15th Edition by E. W. Martin (Mark Publishing Company, 1975), discloses typical carriers and methods of preparation. The compound may also be encapsulated in suitable biocompatible microcapsules, microparticles, nanoparticles, or microspheres formed of biodegradable or non-biodegradable polymers or proteins or liposomes for targeting to cells. Such systems are well known to those skilled in the art and may be optimized for use with the appropriate nucleic acid.

Various methods for nucleic acid delivery are described, for example, in Sambrook et al., Molecular Cloning: A Laboratory Manual, Cold Spring Harbor Laboratory, New York (1989); and Ausubel, et al., Current Protocols in Molecular Biology, John Wiley & Sons, New York (1994). Such nucleic acid delivery systems include the desired nucleic acid, by way of example and not by limitation, in either “naked” form as a “naked” nucleic acid, or formulated in a vehicle suitable for delivery, such as in a complex with a cationic molecule or a liposome forming lipid, or as a component of a vector, or a component of a pharmaceutical composition. The nucleic acid delivery system can be provided to the cell either directly, such as by contacting it with the cell, or indirectly, such as through the action of any biological process. The nucleic acid delivery system can be provided to the cell by endocytosis, receptor targeting, coupling with native or synthetic cell membrane fragments, physical means such as electroporation, combining the nucleic acid delivery system with a polymeric carrier such as a controlled release film or nanoparticle or microparticle, using a vector, injecting the nucleic acid delivery system into a tissue or fluid surrounding the cell, simple diffusion of the nucleic acid delivery system across the cell membrane, or by any active or passive transport mechanism across the cell membrane. Additionally, the nucleic acid delivery system can be provided to the cell using techniques such as antibody-related targeting and antibody-mediated immobilization of a viral vector.

Formulations for topical administration may include ointments, lotions, creams, gels, drops, suppositories, sprays, liquids and powders. Conventional pharmaceutical carriers, aqueous, powder or oily bases, or thickeners can be used as desired.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions, solutions or emulsions that can include suspending agents, solubilizers, thickening agents, dispersing agents, stabilizers, and preservatives. Formulations for injection may be presented in unit dosage form, e.g., in ampules or in multi-dose containers, optionally with an added preservative. The compositions may take such forms as sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, and electrolyte replenishers (such as those based on Ringer's dextrose). Preservatives and other additives may also be present such as, for example, antimicrobials, antioxidants, chelating agents and inert gases. In addition, sterile, fixed oils are conventionally employed as a solvent or suspending medium. For this purpose any bland fixed oil including synthetic mono- or di-glycerides may be employed. In addition, fatty acids such as oleic acid may be used in the preparation of injectables. Carrier formulation can be found in Remington's Pharmaceutical Sciences, Mack Publishing Co., Easton, Pa. Those of skill in the art can readily determine the various parameters for preparing and formulating the compositions without resort to undue experimentation.

The disclosed compositions alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and air. For administration by inhalation, the compounds are delivered in the form of an aerosol spray presentation from pressurized packs or a nebulizer, with the use of a suitable propellant.

In some embodiments, the compositions include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers. In one embodiment, the triplex-forming molecules and/or donor oligonucleotides are conjugated to lipophilic groups like cholesterol and lauric and lithocholic acid derivatives with C32 functionality to improve cellular uptake. For example, cholesterol has been demonstrated to enhance uptake and serum stability of siRNA in vitro (Lorenz, et al., Bioorg. Med. Chem. Lett., 14(19):4975-4977 (2004)) and in vivo (Soutschek, et al., Nature, 432(7014):173-178 (2004)). In addition, it has been shown that binding of steroid conjugated oligonucleotides to different lipoproteins in the bloodstream, such as LDL, protect integrity and facilitate biodistribution (Rump, et al., Biochem. Pharmacol., 59(11):1407-1416 (2000)). Other groups that can be attached or conjugated to the compound described above to increase cellular uptake, include acridine derivatives; cross-linkers such as psoralen derivatives, azidophenacyl, proflavin, and azidoproflavin; artificial endonucleases; metal complexes such as EDTA-Fe(II) and porphyrin-Fe(II); alkylating moieties; nucleases such as alkaline phosphatase; terminal transferases; abzymes; cholesteryl moieties; lipophilic carriers; peptide conjugates; long chain alcohols; phosphate esters; radioactive markers; non-radioactive markers; carbohydrates; and polylysine or other polyamines U.S. Pat. No. 6,919,208 to Levy, et al., also describes methods for enhanced delivery. These pharmaceutical formulations may be manufactured in a manner that is itself known, e.g., by means of conventional mixing, dissolving, granulating, levigating, emulsifying, encapsulating, entrapping or lyophilizing processes.

b. Methods of Administration

In general, methods of administering compounds, including oligonucleotides and related molecules, are well known in the art. In particular, the routes of administration already in use for nucleic acid therapeutics, along with formulations in current use, provide preferred routes of administration and formulation for the triplex-forming molecules described above. Preferably the compositions are injected into the organism undergoing genetic manipulation, such as an animal requiring gene therapy.

The disclosed compositions can be administered by a number of routes including, but not limited to, oral, intravenous, intraperitoneal, intramuscular, transdermal, subcutaneous, topical, sublingual, rectal, intranasal, pulmonary, and other suitable means. The compositions can also be administered via liposomes. Such administration routes and appropriate formulations are generally known to those of skill in the art.

Administration of the formulations may be accomplished by any acceptable method which allows the gene editing compositions to reach their targets.

Any acceptable method known to one of ordinary skill in the art may be used to administer a formulation to the subject. The administration may be localized (i.e., to a particular region, physiological system, tissue, organ, or cell type) or systemic, depending on the condition being treated.

Injections can be e.g., intravenous, intradermal, subcutaneous, intramuscular, or intraperitoneal. In some embodiments, the injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the composition with an aerosol in an inhaler, either alone or attached to a carrier that can be absorbed. For systemic administration, it may be preferred that the composition is encapsulated in liposomes.

The compositions may be delivered in a manner which enables tissue-specific uptake of the agent and/or nucleotide delivery system. Techniques include using tissue or organ localizing devices, such as wound dressings or transdermal delivery systems, using invasive devices such as vascular or urinary catheters, and using interventional devices such as stents having drug delivery capability and configured as expansive devices or stent grafts.

The formulations may be delivered using a bioerodible implant by way of diffusion or by degradation of the polymeric matrix. In certain embodiments, the administration of the formulation may be designed so as to result in sequential exposures to the composition, over a certain time period, for example, hours, days, weeks, months or years. This may be accomplished, for example, by repeated administrations of a formulation or by a sustained or controlled release delivery system in which the compositions are delivered over a prolonged period without repeated administrations. Administration of the formulations using such a delivery system may be, for example, by oral dosage forms, bolus injections, transdermal patches or subcutaneous implants. Maintaining a substantially constant concentration of the composition may be preferred in some cases.

Other delivery systems suitable include time-release, delayed release, sustained release, or controlled release delivery systems. Such systems may avoid repeated administrations in many cases, increasing convenience to the subject and the physician. Many types of release delivery systems are available and known to those of ordinary skill in the art. They include, for example, polymer-based systems such as polylactic and/or polyglycolic acids, polyanhydrides, polycaprolactones, copolyoxalates, polyesteramides, polyorthoesters, polyhydroxybutyric acid, and/or combinations of these. Microcapsules of the foregoing polymers containing nucleic acids are described in, for example, U.S. Pat. No. 5,075,109. Other examples include non-polymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. Specific examples include erosional systems in which the oligonucleotides are contained in a formulation within a matrix (for example, as described in U.S. Pat. Nos. 4,452,775, 4,675,189, 5,736,152, 4,667,013, 4,748,034 and 5,239,660), or diffusional systems in which an active component controls the release rate (for example, as described in U.S. Pat. Nos. 3,832,253, 3,854,480, 5,133,974 and 5,407,686). The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the triplex-forming molecules and donor oligonucleotides. In addition, a pump-based hardware delivery system may be used to deliver one or more embodiments.

Examples of systems in which release occurs in bursts include systems in which the composition is entrapped in liposomes which are encapsulated in a polymer matrix, the liposomes being sensitive to specific stimuli, e.g., temperature, pH, light or a degrading enzyme and systems in which the composition is encapsulated by an ionically-coated microcapsule with a microcapsule core degrading enzyme. Examples of systems in which release of the inhibitor is gradual and continuous include, e.g., erosional systems in which the composition is contained in a form within a matrix and effusional systems in which the composition permeates at a controlled rate, e.g., through a polymer. Such sustained release systems can be in the form of pellets, or capsules.

Use of a long-term release implant may be particularly suitable in some embodiments. “Long-term release,” as used herein, means that the implant containing the composition is constructed and arranged to deliver therapeutically effective levels of the composition for at least 30 or 45 days, and preferably at least 60 or 90 days, or even longer in some cases. Long-term release implants are well known to those of ordinary skill in the art, and include some of the release systems described above.

Suitable subjects include, but are not limited to mammals such as a human or other primate, a rodent such as a mouse or rat, or an agricultural or domesticated animal such as a dog, cat, cow, horse, pig, or sheep. The subject can be an adult, child, infant, or a multi-cell or single-cell embryo. The methods can include in utero delivery of the composition to an embryo or fetus in need thereof.

The in utero methods typically include in utero administration to an embryo or fetus of an effective amount of gene editing composition. Routes of administration include traditional routes such as to intramuscular, intraperitoneal, spinal canal, lumina, lateral cerebral ventricles, puncture of the fetal heart, placental cord insertion, the intrahepatic umbilical vein, intraplacental, yolk sac vessels, intra-organ (e.g., other organs and tissues, including brain, muscle, heart, etc.) and other disclosed herein and in Waddington, et al., “In Utero gene therapy: current challenges and perspectives,” Molecular Therapy, Volume 11, Issue 5, May 2005, Pages 661-676.

In some embodiments the route of administration is via an intravenous or intra-amniotic injection or infusion. The compositions can be administered during in utero surgery. Thus, the methods can used to deliver effective amounts of compositions to the embryo or fetus, or cells thereof, without delivering an effective amount of the composition of the mother of the embryo or fetus, or her cells. For example, in some gene editing embodiments, the target embryo or fetus is contacted with an effective amount of the composition to alter the genomes of a sufficient number of its cells to reduce or prevent one or more symptoms of a target genetic disease. At the same time, the amount, route of delivery, or combination thereof may not be effective to alter genome of a sufficient number of the mother's cells to change her phenotype.

In some methods the compositions can be administered by injection or infusion intravascularly into the vitelline vein, or umbilical vein, or an artery such as the vitelline artery of an embryo or fetus. Additionally (to injection into the vitelline vein) or alternatively, the same or different compositions can be administered by injection or infusion into the amniotic cavity. During physiologic mammalian fetal development, the fetus breaths amniotic fluid into and out of the developing lungs, providing the necessary forces to direct lung development and growth. Developing fetuses additionally swallow amniotic fluid, which aids the formation of the gastrointestinal tract. Introduction of a nanoparticulate composition into the amniotic fluid at gestational ages after the onset of fetal breathing and swallowing resulted in delivery to the lung and gut, respectively, with increased intensity of accumulation at the later gestational ages, while administration before the onset of fetal breathing and swallowing did not lead to any detectable particle accumulation within the fetus.

The methods can be carried out at any time it is technically feasible to do so and the method are efficacious.

In a human, the process of injection can be performed in a manner similar to amniocentesis, during which an ultrasound-guided needle is inserted into the amniotic sac to withdraw a small amount of amniotic fluid for genetic testing. A glass pipette is an exemplary needle-like tool amenable for shape and size modification for piercing through the amniotic membrane via a tiny puncture, and dispensing formulation into the uterus.

The composition can be administered to a fetus, embryo, or to the mother or other subject when the fetus or embryo is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 weeks of age.

In some embodiments, the methods are carried out at a gestational time point during which agents can be safely delivered via the umbilical vessels. In some methods in utero administration is carried out on or after the gestational equivalent of E1S, E15.5, or E16 of a mouse (e.g., a human or mammal's gestational age equivalent to murine gestational age E15, E15.5, or E16). Typically intraamniotic injection is carried out on or after the gestational equivalent of E16 or E16.5, or on or after fetal breathing and/or swallowing has begun.

In other embodiments, intraamniotic injection is carried out on or after the gestational equivaltent of E14, E15, E16, E17, E18, E19, E20, or E21 of a rat (e.g., a human or other mammal's gestational age equivalent to rat gestational age E14, E15, E16, E17, E18, E19, E20, or E21).

c. Preferred Formulations for Mucosal and Pulmonary Administration

Active agent(s) and compositions thereof can be formulated for pulmonary or mucosal administration. The administration can include delivery of the composition to the lungs, nasal, oral (sublingual, buccal), vaginal, or rectal mucosa.

In one embodiment, the compounds are formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorption occurs. The alveoli are covered by a thin epithelium without cilia or a mucus blanket and secrete surfactant phospholipids. The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchiole, which then lead to the ultimate respiratory zone, the alveoli, or deep lung. The deep lung, or alveoli, is the primary target of inhaled therapeutic aerosols for systemic drug delivery.

Pulmonary administration of therapeutic compositions comprised of low molecular weight drugs has been observed, for example, beta-androgenic antagonists to treat asthma. Other therapeutic agents that are active in the lungs have been administered systemically and targeted via pulmonary absorption. Nasal delivery is considered to be a promising technique for administration of therapeutics for the following reasons: the nose has a large surface area available for drug absorption due to the coverage of the epithelial surface by numerous microvilli, the subepithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm³, porous endothelial basement membrane, and it is easily accessible.

The term aerosol as used herein refers to any preparation of a fine mist of particles, which can be in solution or a suspension, whether or not it is produced using a propellant. Aerosols can be produced using standard techniques, such as ultrasonication or high-pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of the compounds in the lungs and that the excipients that are present are present in amount that do not adversely affect uptake of compounds in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, Calif.).

B. Subjects to be Treated

1. Target Diseases

The disclosed compositions can be used for gene therapy. Gene therapy includes, but is not limited to, human genetic diseases, for example, cystic fibrosis, hemophilia, globinopathies such as sickle cell anemia and beta-thalassemia, xeroderma pigmentosum, and lysosomal storage diseases, though the strategies are also useful for treating diseases such as HIV that are not classically considered genetic diseases, in the context of ex vivo-based cell modification and also for in vivo cell modification. The compositions are especially useful to treat genetic deficiencies, disorders and diseases caused by mutations in single genes, for example, to correct genetic deficiencies, disorders and diseases caused by point mutations. If the target gene contains a mutation that is the cause of a genetic disorder, then the compositions can be used for mutagenic repair that may restore the DNA sequence of the target gene to normal. The target sequence can be within the coding DNA sequence of the gene or within an intron. The target sequence can also be within DNA sequences that regulate expression of the target gene, including promoter or enhancer sequences.

If the target gene is an oncogene causing unregulated proliferation, such as in a cancer cell, then the oligonucleotide is useful for causing a mutation that inactivates the gene and terminates or reduces the uncontrolled proliferation of the cell. The oligonucleotide is also a useful anti-cancer agent for activating a repressor gene that has lost its ability to repress proliferation. The target gene can also be a gene that encodes an immune regulatory factor, such as PD-1, in order to enhance the host's immune response to a cancer.

Programmed cell death protein 1, also known as PD-1 and CD279 (cluster of differentiation 279), is a protein encoded by the PDCD1 gene. PD-1 has two ligands: PD-L1 and PD-L2. PD-1 is expressed on a subset of thymocytes and up-regulated on T, B, and myeloid cells after activation (Agata, et al., Int. Immunol., 8:765-772 (1996)). PD-1 acts to antagonize signal transduction downstream of the TCR after it binds a peptide antigen presented by the major histocompatibility complex (MHC). It can function as an immune checkpoint, by preventing the activation of T-cells, which in turn reduces autoimmunity and promotes self-tolerance, but can also reduce the body's ability to combat cancer. The inhibitory effect of PD-1 to act through twofold mechanism of promoting apoptosis (programmed cell death) in antigen specific T-cells in lymph nodes while simultaneously reducing apoptosis in regulatory T cells (suppressor T cells). Compositions that block PD-1, the PD-1 inhibitors, activate the immune system to attack tumors and are therefore used with varying success to treat some types of cancer.

Therefore, in some embodiments, compositions are used to treat cancer. The gene modification technology can be designed to reduce or prevent expression of PD-1, and administered in an effective amount to do so.

The compositions can be used as antiviral agents, for example, when designed to modify a specific a portion of a viral genome necessary for proper proliferation or function of the virus.

Candidates for in utero gene therapy include diseases corrected by replacement of an inactive or absent protein. Monogenic diseases that pose the risk of serious fetal, neonatal, and pediatric morbidity or mortality are particularly attractive targets for in utero gene editing. Exemplary disease targets include, but are not limited to, cystic fibrosis, Tay-Sachs disease, hematopoietic stem cell disorders (e.g., sickle cell, thalassemia), and others disclosed herein. Attractive targets for in utero gene therapy also include those discussed in Schneider & Coutelle, Nature Medicine, 5, 256-257 (1999).

2. Variants, Substitutions, and Exemplary PNAs

Preferred diseases and sequences of exemplary targeting sites, triplex forming molecules, and donor oligonucleotides are discussed in more detail below. Any of the sequences can also be modified as disclosed herein or otherwise known in the art. For example, in some embodiments, any of the triplex-forming molecules herein can have one or more mutations (e.g., substitutions, deletions, or insertions), such that the triplex-forming molecules still bind to the target sequence.

Any of the triplex-forming molecules herein can be manufactured using canonical nucleic acids or other suitable substitutes including those disclosed herein (e.g., PNAs), without or without any of the base, sugar, or backbone modifications discussed herein or in WO 1996/040271, WO/2010/123983, U.S. Pat. No. 8,658,608, WO 2017/143042, and WO 2018/187493.

The triplex-forming molecules herein are typically peptide nucleic acids. In some embodiments, one or more of the cytosines of any of triplex-forming molecules herein is substituted with a pseudoisocytosine. In some embodiments, all of the cytosines in the Hoogsteen binding portion of a triplex forming molecule are substituted with pseudoisocytosine. In some embodiments, any of the triplex-forming molecules herein, includes one or more of peptide nucleic acid residues substituted with a side chain (for example: amino acid side chain or serine side chain) at the alpha, beta and/or gamma position of the backbone. For example, the PNA oligomer can comprise at least one residue comprising a gamma modification/substitution of a backbone carbon atom. In some embodiments all of the peptide nucleic acid residues in the Hoogsteen binding portion only, the Watson-Crick binding portion only, or across the entire PNA are substituted with γPNA residues. In particular embodiments, alternating residues are PNA and γPNA in the Hoogsteen binding portion only, the Watson-Crick binding portion only, or across the entire PNA are substituted. The compositions typically include one ^{ser}γPNA residues, and optionally one or more additional or alternative modifications, for example, miniPEG γPNA residues, methyl γPNA residues, or another y substitution discussed above. In some embodiments, the PNA oligomer includes two or more different modifications of the backbone (e.g. two different types of gamma side chains).

In some embodiments, (1) some or all of the residues in the Watson-Crick binding portion are γPNA residues (e.g., hydroxymethyl-containing γPNA residues); (2) some or all of the residues in the Hoogsteen binding portion are γPNA residues (e.g., hydroxymethyl-containing γPNA residues); or (3) some or all of the residues (in the Watson-Crick and/or Hoogsteen binding portions) are γPNA residues (e.g., hydroxymethyl-containing γPNA residues). Therefore, in some embodiments any of the triplex forming molecules herein is a peptide nucleic acid wherein (1) all of the residues in the Watson-Crick binding portion are γPNA residues (e.g., hydroxymethyl-containing γPNA residues) and none of the residues is in Hoogsteen binding portion are γPNA residues; (2) all of the residues in the Hoogsteen binding portion are γPNA residues (e.g., hydroxymethyl-containing γPNA residues) and none of the residues is in Watson-Crick binding portion are γPNA residues; or (3) all of the residues (in the Watson-Crick and Hoogsteen binding portions) are γPNA residues (e.g., hydroxymethyl-containing γPNA residues). In some embodiments, an integer number between 1 and 50 inclusive PNA residues are hydroxymethyl -containing γPNA residues.

In some embodiments, the triplex-forming molecules are bis-peptide nucleic acids or tail-clamp PNAs with pseudoisocytosine substituted for one or more cytosines, particularly in the Hoogsteen binding portion, and wherein some or all of the PNA residues are γPNA residues.

Any of the triplex-forming molecules herein can have one or more G-clamp-containing residues. For example, one or more cytosines or variant thereof such as pseudoisocytosine in any of the triplex-forming molecules herein can be substituted or otherwise modified to be a clamp-G (9-(2-guanidinoethoxy) phenoxazine).

Any of the triplex-forming molecules herein can include a flexible linker, linking, for example, a Hoogsteen binding domain and a Watson-Crick binding domain to form a bis-PNA or tcPNA. The sequences can be linked with a flexible linker. For example, in some embodiments the flexible linker includes about 1-10, more preferably 2-5, most preferably about 3 units such as 8-amino-2, 6, 10-trioxaoctanoic acid residues. Some molecules include N-terminal or C-terminal non-binding residues, preferably positively charged residues. For example, some molecules include 1-10, preferably 2-5, most preferably about 3 lysines at the N-terminus, the C-terminus, or at both the N-terminus and the C-terminus.

For the disclosed sequences, “J” is pseudoisocytosine, “0” can be a flexible 8-amino-3,6-dioxaoctanoic acid, 6-aminohexanoic acid, 8-amino-2, 6, 10-trioxaoctanoic acid moiety, or 11-amino-3,6,9-trioxaundecanoic acid, and “K” and “lys” (or “Lys”) are lysine.

PNA oligomer sequences are generally presented in N-terminal-to-C-terminal orientation.

In some embodiments, PNA oligomer sequences can be presented in the form: H-“nucleobase sequence”-NH₂ orientation, wherein the H represents the N-terminal hydrogen atom of an unmodified PNA oligomer and the —NH₂ represents the C-terminal amide of the polymer. For bis-PNA and tcPNA, the Hoosten-binding portion can be oriented up stream (e.g., at the “H” or N-terminal end of the polyamide) of the linker, while the Watson-Crick binding portion can be oriented downstream (e.g., at the NH₂ (C-terminal) end) of the polymer/linker.

Any of the donor oligonucleotides can include optional phosphorothioate internucleoside linkages, particular between the two, three or four terminal 5′ and two, three or four terminal 3′ nucleotides. In some embodiments, the phosphorothioate internucleotide linkages need not be sequential and can be dispersed within the donor oligonucleotide. Nevertheless, the phosphorothioate internucleotide linkages can be oriented primarily near each termini of the donor oligonucleotide. Thus, each of the donor oligonucleotide sequences disclosed herein is expressly disclosed without any phosphorothioate internucleoside linkages, and with phosphorothioate internucleoside linkages, preferably between the two, three or four terminal 5′ and two, three or four terminal 3′ nucleotides.

3. Globinopathies

Worldwide, globinopathies account for significant morbidity and mortality. Over 1,200 different known genetic mutations affect the DNA sequence of the human alpha-like (HBZ, HBA2, HBA1, and HBQ1) and beta-like (HBE1, HBG1, HBD, and HBB) globin genes. Two of the more prevalent and well-studied globinopathies are sickle cell anemia and β-thalassemia. Substitution of valine for glutamic acid at position 6 of the β-globin chain in patients with sickle cell anemia predisposes to hemoglobin polymerization, leading to sickle cell rigidity and vasoocclusion with resulting tissue and organ damage. In patients with β-thalassemia, a variety of mutational mechanisms results in reduced synthesis of β-globin leading to accumulation of aggregates of unpaired, insoluble α-chains that cause ineffective erythropoiesis, accelerated red cell destruction, and severe anemia.

Together, globinopathies represent the most common single-gene disorders in man. Triplex forming molecules are particularly well suited to treat globinopathies, as they are single gene disorders caused by point mutations. Triplex forming molecules are effective at binding to the human β-globin both in vitro and in living cells, both ex vivo and in vivo (including by in utero application) in animals. Experimental results also demonstrate correction of a thalassemia-associated mutation in vivo in a transgenic mouse carrying a human beta globin gene with the IVS2-654 thalassemia mutation (in place of the endogenous mouse beta globin) with correction of the mutation in 4% of the total bone marrow cells, cure of the anemia with blood hemoglobin levels showing a sustained elevation into the normal range, reversal of extramedullary hematopoiesis and reversal of splenomegaly, and reduction in reticulocyte counts, following systemic administration of PNA and DNA containing nanoparticles.

β-thalassemia is an unstable hemoglobinopathy leading to the precipitation of α-hemoglobin within RBCs resulting in a severe hemolytic anemia. Patients experience jaundice and splenomegaly, with substantially decreased blood hemoglobin concentrations necessitating repeated transfusions, typically resulting in severe iron overload with time. Cardiac failure due to myocardial siderosis is a major cause of death from β-thalassemia by the end of the third decade. Reduction of repeated blood transfusions in these patients is therefore of primary importance to improve patient outcomes.

a. Exemplary β-globin Gene Target Sites

In the β-globin gene sequence, particularly in the introns, there are many good third-strand binding sites that may be utilized in the methods disclosed herein. A portion of the GenBank sequence of the chromosome-11 human-native hemoglobin-gene cluster (GenBank: U01317.1—Human beta globin region on chromosome 11—LOCUS HUMHBB, 73308 bp ds-DNA) from base 60001 to base 66060 is presented below. Exemplary triplex forming molecule binding sites, are provided in, for example, WO 1996/040271, WO/2010/123983, U.S. Pat. No. 8,658,608, WO 2017/143042, and WO 2018/187493.

b. Exemplary Triplex Forming Sequences

i. Beta Thalassemia

Gene editing molecules can be designed based on the guidance provided herein and otherwise known in the art. Exemplary triplex forming molecule and donor sequences, are provided in, for example, WO 1996/040271, WO/2010/123983, U.S. Pat. No. 8,658,608, WO 2017/143042, and WO 2018/187493 and in the working Examples below, and can be altered to include one or more of the modifications disclosed herein.

In some embodiments, the triplex-forming molecules can form a triple-stranded molecule with the sequence including GAAAGAAAGAGA (SEQ ID NO:7) or TGCCCTGAAAGAAAGAGA (SEQ ID NO:8) or GGAGAAA or AGAATGGTGCAAAGAGG (SEQ ID NO:9) or AAAAGGG or ACATGATTAGCAAAAGGG (SEQ ID NO:10).

Accordingly, in some embodiments, the triplex-forming molecule is a peptide nucleic acid oligomer including the nucleic acid sequence CTTTCTTTCTCT (SEQ ID NO:11), and preferably includes the sequence CTTTCTTTCTCT (SEQ ID NO:11) linked to the sequence TCTCTTTCTTTC (SEQ ID NO:12), or more preferably includes the sequence CTTTCTTTCTCT (SEQ ID NO:11) linked to the sequence TCTCTTTCTTTCAGGGCA (SEQ ID NO:13), and one or more of the peptide nucleic acid residues is a ^{ser}γPNA residue.

In some embodiments, the triplex-forming molecule is peptide nucleic acid oligomer that includes the nucleic acid sequence TTTCCC, preferably includes the sequence TTTCCC linked to the sequence CCCTTTT, or more preferably includes the sequence TTTCCC linked to the sequence CCCTTTTGCTAATCATGT (SEQ ID NO:14),

and one or more of the peptide nucleic acid residues is a ^{ser}γPNA residue.

In some embodiments, the triplex-forming molecule is a peptide nucleic acid oligomer that includes the nucleic acid sequence TTTCTCC, preferably includes the sequence TTTCTCC linked to the sequence CCTCTTT, or more preferably includes the sequence TTTCTCC linked to the sequence CCTCTTTGCACCATTCT (SEQ ID NO:15),

and one or more of the peptide nucleic acid residues is a ^{ser}γPNA residue.

In some preferred embodiments, the triplex forming nucleic acid is a peptide nucleic acid oligomer including the sequence JTTTJTTTJTJT (SEQ ID NO:85) linked to the sequence TCTCTTTCTTTC (SEQ ID NO:12) or TCTCTTTCTTTCAGGGCA (SEQ ID NO:13); or

a peptide nucleic acid oligomer including the sequence TTTTJJJ linked to the sequence CCCTTTT or CCCTTTTGCTAATCATGT (SEQ ID NO:14);

or a peptide nucleic acid oligomer including the sequence TTTJTJJ linked to the sequence CCTCTTT or CCTCTTTGCACCATTCT (SEQ ID NO:15);

wherein one or more of the PNA residues is a ^{ser}γPNA.

In specific embodiments, the triplex forming molecule is a peptide nucleic acid oligomer including the sequence lys-lys-lys-JTTTJTTTJTJT-OOO-TCTCTTTCTTTCAGGGCA-lys-lys-lys (SEQ ID NO:16), or

lys-lys-lys-TTTTJJJ-OOO-CCCTTTTGCTAATCATGT-lys-lys-lys (SEQ ID NO:17), or

lys-lys-lys-TTTJTJJ-OOO-CCTCTTTGCACCATTCT-lys-lys-lys (SEQ ID NO:18);

wherein one or more of the PNA residues is a ^{ser}γPNA. In even more specific embodiments, the bolded and underlined residues are ^{ser}γPNA residues.

In other embodiments, the triplex forming nucleic acid is a peptide nucleic acid oligomer including the sequence TJTTTTJTTJ (SEQ ID NO:19) linked to the sequence CTTCTTTTCT (SEQ ID NO:20); or

TTJTTJTTTJ (SEQ ID NO:21) linked to the sequence CTTTCTTCTT (SEQ ID NO:22); or

JJJTJJTTJT (SEQ ID NO:23) linked to the sequence TCTTCCTCCC (SEQ ID NO:24); or

wherein one or more of the PNA residues is a ^{ser}γPNA.

In specific embodiments, the triplex forming nucleic acid is a peptide nucleic acid oligomer including the sequence lys-lys-lys-TJTTTTJTTJ-OOO-CTTCTTTTCT-lys-lys-lys (SEQ ID NO:25) (IVS2-24); or

lys-lys-lys-TTJTTJTTTJ-OOO-CTTTCTTCTT-lys-lys-lys (SEQ ID NO:26) (IVS2-512); or

lys-lys-lys-JJJTJJTTJT-OOO-TCTTCCTCCC-lys-lys-lys (SEQ ID NO:27) (IVS2-830);

wherein one or more of the PNA residues is a ^{ser}γPNA. In even more specific embodiments, the bolded and underlined residues are ^{ser}γPNA residues.

ii. Sickle Cell Disease

Preferred sequences that target the sickle cell disease mutation (20) in the beta globin gene are also provided. In some embodiments, the triplex-forming molecule is a peptide nucleic acid oligomer that includes the nucleic acid sequence CCTCTTC, preferably includes the sequence CCTCTTC linked to the sequence CTTCTCC, or more preferably includes the sequence CCTCTTC linked to the sequence CTTCTCCAAAGGAGT (SEQ ID NO:28) or CTTCTCCACAGGAGTCAG (SEQ ID NO:29) or CTTCTCCACAGGAGTCAGGTGC (SEQ ID NO:30),

and one or more of the peptide nucleic acid residues is a ^{ser}γPNA residue.

In some embodiments, the triplex-forming molecule is a peptide nucleic acid oligomer that includes the nucleic acid sequence TTCCTCT, preferably includes the sequence TTCCTCT linked to the sequence TCTCCTT, or more preferably includes the sequence TTCCTCT linked to the sequence TCTCCTTAAACCTGT (SEQ ID NO:31) or TCTCCTTAAACCTGTCTT (SEQ ID NO:32),

and one or more of the peptide nucleic acid residues is a ser_{y}PNA residue.

In some embodiments, the triplex-forming molecule is a peptide nucleic acid oligomer that includes the nucleic acid sequence TCTCTTCT, preferably includes the sequence TCTCTTCT linked to the sequence TCTTCTCT, or more preferably includes the sequence TCTCTTCT linked to the sequence TCTTCTCTGTCTCCAC (SEQ ID NO:33) or TCTTCTCTGTCTCCACAT (SEQ ID NO:34),

and one or more of the peptide nucleic acid residues is a ser_{y}PNA residue.

In some preferred embodiments for correction of Sickle Cell Disease Mutation, the triplex forming nucleic acid is a peptide nucleic acid oligomer including the sequence JJTJTTJ linked to the sequence CTTCTCC or CTTCTCCAAAGGAGT (SEQ ID NO:28) or CTTCTCCACAGGAGTCAG (SEQ ID NO:29) or CTTCTCCACAGGAGTCAGGTGC (SEQ ID NO:30);

or a peptide nucleic acid oligomer including the sequence TTJJTJT linked to the sequence TCTCCTT or TCTCCTTAAACCTGT (SEQ ID NO:31) or TCTCCTTAAACCTGTCTT (SEQ ID NO:32);

or a peptide nucleic acid including the sequence TJTJTTJT linked to the sequence TCTTCTCT or TCTTCTCTGTCTCCAC (SEQ ID NO:33) or TCTTCTCTGTCTCCACAT (SEQ ID NO:34);

wherein one or more of the PNA residues is a ^{ser}γPNA.

In specific embodiments for correction of Sickle Cell Disease Mutation, the triplex forming nucleic acid is a peptide nucleic acid oligomer including the sequence

(SEQ ID NO: 35)
lys-lys-lys-JJTJTTJ-OOO-C [symbol] T [symbol] T [symbol] C [symbol] A [symbol] G [symbol] A [symbol] T-
lys-lys-lys;
or
(SEQ ID NO: 36)
lys-lys-lys-TTJJTJT-OOO-T [symbol] T [symbol] C [symbol] T [symbol] A [symbol] C [symbol] T [symbol] T-
lys-lys-lys;
or
(SEQ ID NO: 37)
lys-lys-lys-TTJJTJT-OOO-T [symbol] T [symbol] C [symbol] T [symbol] A [symbol] C [symbol] T [symbol] T [symbol]
T [symbol] -lys-lys-lys
(SEQ ID NO: 38)
lys-lys-lys-TJTJTTJT-OOO-T [symbol] T [symbol] C [symbol] C [symbol] G [symbol] C [symbol] C [symbol] A [symbol]
-lys-lys-lys (tc816);
or
(SEQ ID NO: 39)
lys-lys-lys-JJTJTTJ-OOO-C [symbol] T [symbol] T [symbol] C [symbol] C [symbol] G [symbol] A [symbol] T [symbol]
A [symbol] -lys-lys-lys;
or
(SEQ ID NO: 39)
lys-lys-lys-JJTJTTJ-OOO- [symbol] T [symbol] C [symbol] C [symbol] A [symbol] A [symbol] G [symbol] G [symbol]
C [symbol] G-lys-lys-lys (SCD-tcPNA 1A);
or
(SEQ ID NO: 39)
lys-lys-lys-JJTJTTJ-OOO- [symbol] [symbol] -lys-
lys-lys (SCD-tcPNA IB);
or
(SEQ ID NO: 39)
lys-lys-lys-JJ [symbol] J [symbol] J-OOO- [symbol] [symbol] -
lys-lys-lys (SCD-tcPNA 1C);
or
(SEQ ID NO: 40)
lys-lys-lys-JJTJTTJ-OOO-CTTCTCCACAGGAGTCAGGTGC-
lys-lys-lys (SCD-tcPNA ID);
or
(SEQ ID NO: 40)
Iys-lys-lys-JJTJTTJ-OOO- [symbol] [symbol] -
lys-lys-lys (SCD-tcPNA IE);
or
(SEQ ID NO: 40)
lys-lys-lys-JJ [symbol] J [symbol] J-OOO- [symbol] [symbol] -
lys-lys-lys (SCD-tcPNA IF);
or
(SEQ ID NO: 41)
lys-lys-lys-TJTJTTJT-OOO-T [symbol] T [symbol] C [symbol] C [symbol] G [symbol] C [symbol] C [symbol] A [symbol]
A [symbol] -lys-lys-lys;

wherein one or more of the PNA residues is a ^{ser}γPNA. In even more specific embodiments, the bolded and underlined residues are ^{ser}γPNA residues.

c. Exemplary Donors

In some embodiments, the triplex forming molecules are used in combination with a donor oligonucleotide for correction of IVS2-654 mutation that includes the sequence 5′AAAGAATAACAGTGATAATTTCTGGGTTAAGGCAATAGCAATA TCTCTGCATATAAATAT 3′ (SEQ ID NO:86) with the correcting IVS2-654 nucleotide underlined, or a functional fragment thereof that is suitable and sufficient to correct the IVS2-654 mutation.

Other exemplary donor sequences include, but are not limited to, DonorGFP-IVS2-1 (Sense) 5′-GTTCAGCGTGTCCGGCGAGGGCG AGGTGAGTCTATGGGACCCTTGATGTTT-3′ (SEQ ID NO:42), DonorGFP-IVS2-1 (Antisense) 5′-AAACATCAAGGGTCCCATA GACTCACCTCGCCCTCGCCGGACACGCTGAAC-3′ (SEQ ID NO:43), and, or a functional fragment thereof that is suitable and sufficient to correct a mutation.

In some embodiments, a Sickle Cell Disease mutation can be corrected using a donor having the sequence

5′CTTGCCCCACAGGGCAGTAACGGCAGATTTTTC [symbol] CGG CGTTAAATGCACCATGGTGTCTGTTTGAGGT 3′ (SEQ ID NO:44), or a functional fragment thereof that is suitable and sufficient to correct a mutation, wherein the three boxed nucleotides represent the corrected codon 6 which reverts the mutant Valine (associated with human sickle cell disease) back to the wildtype Glutamic acid and nucleotides in bold font (without underlining) represent changes to the genomic DNA but not to the encoded amino acid; or

5′ACAGACACCATGGTGCACCTGACTCCTGAGGAGAAGTCT GCCGTTACTGCC 3′ (SEQ ID NO:45), or a functional fragment thereof that is suitable and sufficient to correct a mutation, wherein the bolded and underlined residue is the correction, or

5′T(s)T(s)G(s)CCCCACAGGGCAGTAACGGCAGACTTCTCCTC AGGAGTCAGGTGCACCATGGTGTCTGTT(s)T(s)G(s)3′ (SEQ ID NO:46), or a functional fragment thereof that is suitable and sufficient to correct a mutation, wherein the bolded and underlined residue is the correction and “(s)” indicates an optional phosphorothioate internucleoside linkage.

4. Cystic Fibrosis

The disclosed compositions and methods can be used to treat cystic fibrosis. Cystic fibrosis (CF) is a lethal autosomal recessive disease caused by defects in the cystic fibrosis transmembrane conductance regulator (CFTR), an ion channel that mediates Cl— transport. Lack of CFTR function results in chronic obstructive lung disease and premature death due to respiratory failure, intestinal obstruction syndromes, exocrine and endocrine pancreatic dysfunction, and infertility (Davis, et al., Pediatr Rev., 22(8):257-64 (2001)). The most common mutation in CF is a three base-pair deletion (F508del) resulting in the loss of a phenylalanine residue, causing intracellular degradation of the CFTR protein and lack of cell surface expression (Davis, et al., Am J Respir Crit Care Med., 173(5):475-82 (2006)). In addition to this common mutation there are many other mutations that occur and lead to disease including a class of mutations due to premature stop codons, nonsense mutations. In fact nonsense mutations account for approximately 10% of disease causing mutations. Of the nonsense mutations G542X and W1282X are the most common with frequencies of 2.6% and 1.6% respectfully.

Although CF is one of the most rigorously characterized genetic diseases, current treatment of patients with CF focuses on symptomatic management rather than primary correction of the genetic defect. Gene therapy has remained an elusive target in CF, because of challenges of in vivo delivery to the lung and other organ systems (Armstrong, et al., Archives of disease in childhood (2014) doi: 10.1136/archdischild-2012-302158. PubMed PMID: 24464978). In recent years, there have been many advances in gene therapy for treatment of diseases involving the hematolymphoid system, where harvest and ex vivo manipulation of cells for autologous transplantation is possible: some examples include the use of zinc finger nucleases targeting CCRS to produce HIV-1 resistant cells (Holt, et al., Nature biotechnology, 28(8):839-47 (2010)) correction of the ABCD1 gene by lentiviral vectors for treatment of adrenoleukodystrophy (Cartier, et al., Science, 326(5954):818-23 (2009)) and correction of SCID due to ADA deficiency using retroviral gene transfer (Aiuti, et al., The New England Journal Of Medicine, 360(5):447-58 (2009).

Harvest and autologous transplant is not an option in CF, due to the involvement of the lung and other internal organs. As one approach, the UK Cystic Fibrosis Gene Therapy Consortium has tested liposomes to deliver plasmids containing cDNA encoding CFTR to the lung (Alton, et al., Thorax, 68(11):1075-7 (2013)), Alton, et al., The Lancet Respiratory Medicine, (2015). doi: 10.1016/S2213-2600(15)00245-3. PubMed PMID: 26149841.) other clinical trials have used viral vectors for delivery of the CFTR gene or CFTR expression plasmids that are compacted by polyethylene glycol-substituted lysine 30-mer peptides with limited success (Konstan, et al., Human Gene Therapy, 15(12):1255-69 (2004)). Moreover, delivery of plasmid DNA for gene addition without targeted insertion does not result in correction of the endogenous gene and is not subject to normal CFTR gene regulation, and virus-mediated integration of the CFTR cDNA could introduce the risk of non-specific integration into important genomic sites.

However, it has been discovered that triplex-forming PNA molecules and donor DNA can be used to correct mutations leading to cystic fibrosis. In preferred embodiments, the compositions are administered by intranasal or pulmonary delivery. In some embodiments, the triplex-forming molecules can be administered in utero; for example by amniotic sac injection and/or injection into the vitelline vein. In utero approaches offer several advantages including, for example, the large number of somatic stem cells available for gene correction and a reduced inflammatory response due to the immune-privileged status of the fetus (see, e.g., Larson and Cohen, In Utero Gene Therapy, Ochsner J., 2(2):107-110 (2000)). Other exemplary advantages include stem cells are rapidly dividing, relatively smaller size of the organism compared to mature, adult organisms, a smaller dosage can be effective, therapies can be delivered before or during the pathogenesis of irreversible organ damage, etc.

In CF, for example, there is evidence of significant multisystem organ damage at birth

The compositions can be administered in an effective amount to induce or enhance gene correction in an amount effective to reduce one or more symptoms of cystic fibrosis. For example, in some embodiments, the gene correction occurs at an amount effective to improve impaired response to cyclic AMP stimulation, improve hyperpolarization in response to forskolin, reduction in the large lumen negative nasal potential, reduction in inflammatory cells in the bronchoalveolar lavage (BAL), improve lung histology, or a combination thereof. In some embodiments, the target cells are cells, particularly epithelial cells, that make up the sweat glands in the skin, that line passageways inside the lungs, liver, pancreas, or digestive or reproductive systems. In particular embodiments, the target cells are bronchial epithelial cells. While permanent genomic change using PNA/DNA is less transient than plasmid-based approaches and the changes will be passed on to daughter cells, some modified cells may be lost over time with regular turnover of the respiratory epithelium. In some embodiments, the target cells are lung epithelial progenitor cells. Modification of lung epithelial progenitors can induce more long-term correction of phenotype.

Sequences for the human cystic fibrosis transmembrane conductance regulator (CFTR) are known in the art, see, for example, GenBank Accession number: AH006034.1, and compositions and methods of targeted correction of CFTR are described in McNeer, et al., Nature Communications, 6:6952, (DOI 10.1038/ncomms7952), 11 pages.

a. Exemplary F508del Target Sites

In some embodiments, the triplex-forming molecules are designed to target the CFTR gene at nucleotides 9,152-9,159 (TTTCCTCT) or 9,159-9,168 (TTTCCTCTATGGGTAAG (SEQ ID NO:47) of accession number AH006034.1, or the non-coding strand (e.g., 3′-5′ complementary sequence) corresponding to nucleotides 9,152-9,159 or 9,152-9,168 (e.g., 5′-AGAGGAAA-3′, or 5′-CTTACCCATAGAGGAAA-3′ (SEQ ID NO:48)).

In some embodiments, the triplex-forming molecules are designed to target the CFTR gene at nucleotides 9,039-9,046 (5′-AGAAGAGG-3′), or 9,030-9,046 (5′-ATGCCAACTAGAAGAGG-3′ (SEQ ID NO:49)) of accession number AH006034.1, or the non-coding strand (e.g., 3′-5′ complementary sequence) corresponding to nucleotides (5′ CCTCTTCT 3′) or (5′ CCTCTTCTAGTTGGCAT 3′ (SEQ ID NO:50).

In some embodiments, the triplex-forming molecules are designed to target the CFTR gene at nucleotides 8,665-8,683 (CTTTCCCTT) or 8,665-8,682 (CTTTCCCTTGTATCTTTT (SEQ ID NO:51) of accession number AH006034.1, or the non-coding strand (e.g., 3′-5′ complementary sequence) corresponding to nucleotides 8,665-8,683 or 8,665-8,682 (e.g., 5′-AAGGGAAAG-3′, or 5′-AAAAGATACAAGGGAAAG -3′ (SEQ ID NO:52)).

In some embodiments, the triplex-forming molecules are designed to target the W1282X mutation in CFTR gene at the sequence GAAGGAGAAA (SEQ ID NO:53), AAAAGGAA, or AGAAAAAAGG (SEQ ID NO:55), or the inverse complement thereof.

In some embodiments, the triplex-forming molecules are designed to target the G542X mutation in CFTR gene at the sequence AGAAAAA, AGAGAAAGA, or AAAGAAA, or the inverse complement thereof.

b. Exemplary Triplex Forming Sequences and Donors

i. F508del

In some embodiments, the triplex-forming molecule is a peptide nucleic acid oligomer that includes the nucleic acid sequence includes TCTCCTTT, preferably linked to the sequence TTTCCTCT or more preferably includes TCTCCTTT linked to the sequence TTTCCTCTATGGGTAAG (SEQ ID NO:47); or

includes TCTTCTCC preferably linked to the sequence CCTCTTCT, or more preferably includes TCTTCTCC linked to CCTCTTCTAGTTGGCAT (SEQ ID NO:50); or

includes TTCCCTTTC, preferably includes the sequence TTCCCTTTC linked to the sequence CTTTCCCTT, or more preferably includes the sequence TTCCCTTTC linked to the sequence CTTTCCCTTGTATCTTTT (SEQ ID NO:51);

and one or more of the peptide nucleic acid residues is a ^{ser}γPNA residue.

In some preferred embodiments, the triplex forming nucleic acid is a peptide nucleic acid oligomer including the sequence TJTJJTTT, linked to the sequence TTTCCTCT or TTTCCTCTATGGGTAAG (SEQ ID NO:47); or

TJTTJTJJ linked to the sequence CCTCTTCT, or CCTCTTCTAGTTGGCAT (SEQ ID NO:50); or

TTJJJTTTJ linked to the sequence CTTTCCCTT, or CTTTCCCTTGTATCTTTT (SEQ ID NO:51);

wherein one or more of the PNA residues is a ^{ser}γPNA.

In specific embodiments the triplex forming nucleic acid is a peptide nucleic acid oligomer including the sequence is lys-lys-lys-TJTJJTTT-OOO-TTTCCTCTATGGGTAAG-lys-lys-lys (SEQ ID NO:89) (hCFPNA2); or

lys-lys-lys-TJTJJTTT-OOO-TTTCCTCTATGGGTAAG-lys-lys-lys (SEQ ID NO:89); or

lys-lys-lys-TJTTJTJJ-OOO-CCTCTTCTAGTTGGCAT-lys-lys-lys (SEQ ID NO:90) (hCFPNA1); or

lys-lys-lys-TTJJJTTTJ-OOO-CTTTCCCTTGTATCTTTT-lys-lys-lys (SEQ ID NO:54) (hCFPNA3);

wherein one or more of the PNA residues is a ^{ser}γPNA. In even more specific embodiments, the bolded and underlined residues are ^{ser}γPNA residues.

In some embodiments, a donor that can be used for CFTR gene correction, particularly in combination with the foregoing triplex forming molecules, includes the sequence 5′TTCTGTATCTATATTCATCATAGGAAACACCAAAGATAATGTTCT CCTTAATGGTGCCAGG3′ (SEQ ID NO:91), or a functional fragment thereof that is suitable and sufficient to correct the F508del mutation in the cystic fibrosis transmembrane conductance regulator (CFTR) gene.

ii. W1282 Mutation Site

In some embodiments, the triplex-forming molecule is a peptide nucleic acid oligomer that includes the nucleic acid sequence CTTCCTCTTT (SEQ ID NO:56), preferably includes the sequence CTTCCTCTTT (SEQ ID NO:56) linked to the sequence TTTCTCCTTC (SEQ ID NO:57), or more preferably includes the sequence CTTCCTCTTT (SEQ ID NO:56) linked to the sequence TTTCTCCTTCAGTGTTCA (SEQ ID NO:58); or

includes the nucleic acid sequence TTTTCCT, preferably includes the sequence TTTTCCT linked to the sequence TCCTTTT, or more preferably includes the sequence TTTTCCT linked to the sequence TCCTTTTGCTCACCTGTGGT (SEQ ID NO:59); or

includes the nucleic acid sequence TCTTTTTTCC (SEQ ID NO:60), preferably includes the sequence TCTTTTTTCC (SEQ ID NO:60) linked to the sequence CCTTTTTTCT (SEQ ID NO:61), or more preferably includes the sequence TCTTTTTTCC (SEQ ID NO:60) linked to the sequence CCTTTTTTCTGGCTAAGT (SEQ ID NO:62);

and one or more of the peptide nucleic acid residues is a ser_{y}PNA residue.

In preferred embodiments, the triple forming nucleic acid is a peptide nucleic acid oligomer including the sequence JTTJJTJTTT (SEQ ID NO:63) linked to the sequence TTTCTCCTTC (SEQ ID NO:57) or TTTCTCCTTCAGTGTTCA (SEQ ID NO:58); or

a peptide nucleic acid oligomer including the sequence TTTTJJT linked to the sequence TCCTTTT or linked to the sequence TCCTTTTGCTCACCTGTGGT (SEQ ID NO:59); or

a peptide nucleic acid oligomer including the sequence TJTTTTTTJJ (SEQ ID NO:64) linked to the sequence CCTTTTTTCT (SEQ ID NO:61) or linked to the sequence CCTTTTTTCTGGCTAAGT (SEQ ID NO:62);

wherein one or more of the PNA residues is a ^{ser}γPNA.

In specific embodiments, the triplex forming nucleic acid is a peptide nucleic acid oligomer including the sequence lys-lys-lys-JTTJJTJTTT-OOO-TTTCTCCTTCAGTGTTCA-lys-lys-lys (SEQ ID NO:65) (tcPNA-1236); or

lys-lys-lys-TTTTJJT-OOO-TCCTTTTGCTCACCTGTGGT-lys-lys-lys (SEQ ID NO:66) (tcPNA-1314); or

lys-lys-lys-TJTTTTTTJJ-OOO-CCTTTTTTCTGGCTAAGT-lys-lys-lys (SEQ ID NO:67) (tcPNA-1329);

wherein one or more of the PNA residues is a ^{ser}γPNA. In even more specific embodiments, the bolded and underlined residues are ^{ser}γPNA residues.

In some embodiments, a donor that can be used for CFTR gene correction, particularly in combination with the foregoing triplex forming molecules, includes the sequence T(s)C(s)T(s)-TGGGATTCAATAACCTTGCAGACAGTGGAGGAAGGCCTTTGGCG TGATACCACAGG-(s)T(s)G(s) (SEQ ID NO:68) or a functional fragment thereof that is suitable and sufficient to correct a mutation in CFTR, wherein the bolded and underlined nucleotides are inserted mutations for gene correction, and “(s)” indicates an optional phosphorothioate internucleoside linkage.

iii. G542X Mutation Site

In some embodiments, the triplex-forming molecule is a peptide nucleic acid oligomer that includes the nucleic acid sequence TCTTTTT, preferably includes the sequence TCTTTTT linked to the sequence TTTTTCT, or more preferably includes the sequence TCTTTTT linked to the sequence TTTTTCTGTAATTTTTAA (SEQ ID NO:69); or

includes the nucleic acid sequence TCTCTTTCT, preferably includes the sequence TCTCTTTCT linked to the sequence TCTTTCTCT, or more preferably includes the sequence TCTCTTTCT linked to the sequence TCTTTCTCTGCAAACTT (SEQ ID NO:70); or

includes the nucleic acid sequence TTTCTTT, preferably includes the sequence TTTCTTT linked to the sequence TTTCTTT, or more preferably includes the sequence TTTCTTT linked to the sequence TTTCTTTAAGAACGAGCA (SEQ ID NO:71);

and one or more of the peptide nucleic acid residues is a ^{ser}γPNA residue.

In preferred embodiments, the triple forming nucleic acid is a peptide nucleic acid oligomer including the sequence TJTTTTT linked to the sequence TTTTTCT or TTTTTCTGTAATTTTTAA (SEQ ID NO:69); or

a peptide nucleic acid oligomer including the sequence TJTJTTTJT linked to the sequence TCTTTCTCT or linked to the sequence TCTTTCTCTGCAAACTT (SEQ ID NO:70); or

a peptide nucleic acid oligomer including the sequence TTTJTTT linked to the sequence TTTCTTT or linked to the sequence TTTCTTTAAGAACGAGCA (SEQ ID NO:71);

wherein one or more of the PNA residues is a ^{ser}γPNA.

In specific embodiments, the triplex forming nucleic acid is a peptide nucleic acid oligomer including the sequence lys-lys-lys-TJTTTTT-OOO-TTTTTCTGTAATTTTTAA-lys-lys-lys (SEQ ID NO:72) (tcPNA-302); or

lys-lys-lys-TJTJTTTJT-OOO-TCTTTCTCTGCAAACTT-lys-lys-lys (SEQ ID NO:73) (tcPNA-529); or

lys-lys-lys-TTTJTTT-OOO-TTTCTTTAAGAACGAGCA-lys-lys-lys (SEQ ID NO:74) (tcPNA-586);

wherein one or more of the PNA residues is a ^{ser}γPNA. In even more specific embodiments, the bolded and underlined residues are ^{ser}γPNA residues.

In some embodiments, a donor that can be used for CFTR gene correction, particularly in combination with the foregoing triplex forming molecules, includes the sequence T(s)C(s)C(s)-AAGTTTGCAGAGAAAGATAATATAGTCCTTGGAGAAGGAGGAAT CACCCTGAGTGGA-G(s)G(s)T(s) (SEQ ID NO:75), or a functional fragment thereof that is suitable and sufficient to correct a mutation in CFTR, wherein the bolded and underlined nucleotides are inserted mutations for gene correction, and “(s)” indicates an optional phosphorothioate internucleoside linkage. 5. HIV

The gene editing compositions can be used to treat infections, for example those caused by HIV.

a. Exemplary Target Sites

The target sequence for the triplex-forming molecules is within or adjacent to a human gene that encodes a cell surface receptor for human immunodeficiency virus (HIV). Preferably, the target sequence of the triplex-forming molecules is within or is adjacent to a portion of a HIV receptor gene important to its function in HIV entry into cells, such as sequences that are involved in efficient expression of the receptor, transport of the receptor to the cell surface, stability of the receptor, viral binding by the receptor, or endocytosis of the receptor. Target sequences can be within the coding DNA sequence of the gene or within introns. Target sequences can also be within DNA sequences that regulate expression of the target gene, including promoter or enhancer sequences.

The target sequence can be within or adjacent to any gene encoding a cell surface receptor that facilitates entry of HIV into cells. The molecular mechanism of HIV entry into cells involves specific interactions between the viral envelope glycoproteins (env) and two target cell proteins, CD4 and the chemokine receptors. HIV cell tropism is determined by the specificity of the env for a particular chemokine receptor, a 7 transmembrane-spanning, G protein-coupled receptor (Steinberger, et al., Proc. Natl. Acad. Sci. USA. 97: 805-10 (2000)). The two major families of chemokine receptors are the CXC chemokine receptors and the CC chemokine receptors (CCR) so named for their binding of CXC and CC chemokines, respectively. While CXC chemokine receptors traditionally have been associated with acute inflammatory responses, the CCRs are mostly expressed on cell types found in connection with chronic inflammation and T-cell-mediated inflammatory reactions: eosinophils, basophils, monocytes, macrophages, dendritic cells, and T cells (Nansen, et al. 2002, Blood 99:4). In one embodiment, the target sequence is within or adjacent to the human genes encoding chemokine receptors, including, but not limited to, CXCR4, CCR5, CCR2b, CCR3, and CCR1.

In a preferred embodiment, the target sequence is within or adjacent to the human CCR5 gene. The CCR5 chemokine receptor is the major co-receptor for RS-tropic HIV strains, which are responsible for most cases of initial, acute HIV infection. Individuals who possess a homozygous inactivating mutation, referred to as the 432 mutation, in the CCR5 gene are almost completely resistant to infection by RS-tropic HIV-1 strains. The 432 mutation produces a 32 base pair deletion in the CCR5 coding region.

Another naturally occurring mutation in the CCR5 gene is the m303 mutation, characterized by an open reading frame single T to A base pair transversion at nucleotide 303 which indicates a cysteine to stop codon change in the first extracellular loop of the chemokine receptor protein at amino acid 101 (C101X) (Carrington et al. 1997). Mutagenesis assays have not detected the expression of the m303 co-receptor on the surface of CCR5 null transfected cells which were found to be non-susceptible to HIV-1 R5-isolates in infection assays (Blanpain, et al. (2000).

Compositions and methods for targeted gene therapy using triplex-forming oligonucleotides and peptide nucleic acids for treating infectious diseases such as HIV are described in U.S. Application No. 2008/050920 and WO 2011/133803. Each provides sequences of triplex forming molecules, target sequences, and donor oligonucleotides that can be utilized in the compositions and methods provided herein.

For example, individuals having the homozygous M2 inactivating mutation in the CCR5 gene display no significant adverse phenotypes, suggesting that this gene is largely dispensable for normal human health. This makes the CCR5 gene a particularly attractive target for targeted mutagenesis using the triplex-forming molecules disclosed herein. The gene for human CCR5 is known in the art and is provided at GENBANK accession number NM_000579. The coding region of the human CCR5 gene is provided by nucleotides 358 to 1416 of GENBANK accession number NM_000579.

In some embodiments, the target region is a polypurine site within or adjacent to a gene encoding a chemokine receptor including CXCR4, CCR5, CCR2b, CCR3, and CCR1. In a preferred embodiment, the target region is a polypurine or homopurine site within the coding region of the human CCR5 gene. Three homopurine sites in the coding region of the CCR5 gene that are especially useful as target sites for triplex-forming molecules are from positions 509-518, 679-690 and 900-908 relative to the ATG start codon.

The homopurine site from 679-690 partially encompasses the site of the nonsense mutation created by the Δ32 mutation. Triplex-forming molecules that bind to this target site are particularly useful.

b. Exemplary Triplex Forming Sequences

In some embodiments, the triplex-forming molecule is a peptide nucleic acid oligomer that includes the nucleic acid sequence CTCTTCTTCT (SEQ ID NO:76), preferably includes the sequence CTCTTCTTCT (SEQ ID NO:76) linked to the sequence TCTTCTTCTC (SEQ ID NO:77), or more preferably includes the sequence CTCTTCTTCT (SEQ ID NO:76) linked to the sequence TCTTCTTCTCATTTC (SEQ ID NO:78),

and one or more of the peptide nucleic acid residues is a ^{ser}γPNA residue.

In some embodiments, the triplex-forming molecule is a peptide nucleic acid oligomer that includes the nucleic acid sequence CTTCT, preferably includes the sequence CTTCT linked to the sequence TCTTC or TCTTCTTCTC (SEQ ID NO:77), or more preferably includes the sequence CTTCT linked to the sequence TCTTCTTCTCATTTC (SEQ ID NO:78),

and one or more of the peptide nucleic acid residues is a ^{ser}γPNA residue.

In preferred embodiments, the triplex forming nucleic acid is a peptide nucleic acid oligomer including the sequence JTJTTJTTJT (SEQ ID NO:84) linked to the sequence TCTTCTTCTC (SEQ ID NO:77) or TCTTCTTCTCATTTC (SEQ ID NO:78);

or JTTJT linked to the sequence TCTTC or TCTTCTTCTC (SEQ ID NO:77) or more preferably TCTTCTTCTCATTTC (SEQ ID NO:78);

wherein one or more of the PNA residues is a ^{ser}γPNA.

In specific embodiments, the triplex forming nucleic acid is a peptide nucleic acid oligomer including the sequence Lys-Lys-Lys-JTJTTJTTJT-OOO-TCTTCTTCTCATTTC-Lys-Lys-Lys (SEQ ID NO:79) (PNA-679);

or Lys-Lys-Lys-JTTJT-OOO-TCTTCTTCTCATTTC-Lys-Lys-Lys (SEQ ID NO:80) (tcPNA-684)

wherein one or more of the PNA residues is a ^{ser}γPNA. In even more specific embodiments, the bolded and underlined residues are ^{ser}γPNA residues.

c. Exemplary Donor Sequences

In some embodiments, the triplex forming molecules are used in combination with one or more donor oligonucleotides such as donor 591 having the sequence: 5′ AT TCC CGA GTA GCA GAT GAC CAT GAC AGC TTA GGG CAG GAC CAG CCC CAA GAT GAC TAT C 3′ (SEQ ID NO:81), or donor 597 having the sequence 5′ TT TAG GAT TCC CGA GTA GCA GAT GAC CCC TCA GAG CAG CGG CAG GAC CAG CCC CAA GAT G 3′ (SEQ ID NO:82), which can be used in combination to induce two different non-sense mutations, one in each allele of the CCR5 gene, in the vicinity of the 432 deletion (mutation sites are bolded); or a functional fragment thereof that is suitable and sufficient to introduce a non-sense mutation in at least one allele of the CCR5 gene.

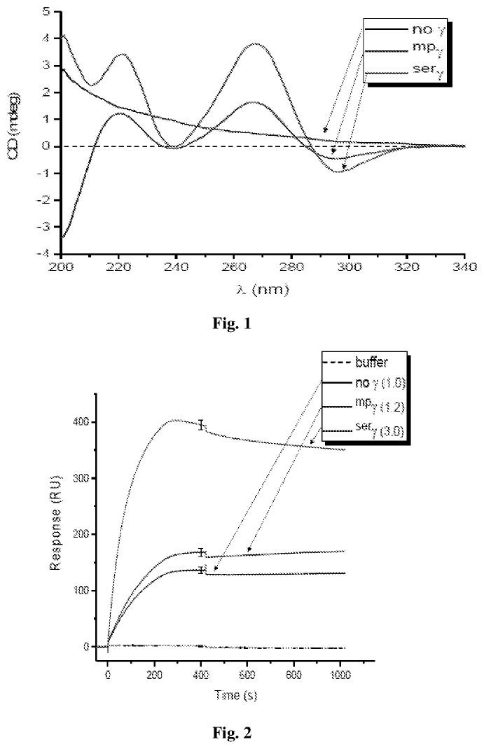

In another preferred embodiment, donor oligonucleotides are designed to span the Δ32 deletion site (see, e.g., FIG. 1 of WO 2011/133803) and induce changes into a wildtype CCR5 allele that mimic the Δ32 deletion. Donor sequences designed to target the Δ32 deletion site may be particularly usefully to facilitate knockout of the single wildtype CCR5 allele in heterozygous cells.

Preferred donor sequences designed to target the Δ32 deletion site include, but are not limited to,

Donor DELTA32JDC:
(SEQ ID NO: 92)
5′GATGACTATCTTTAATGTCTGGAAATTCTTCCAGAATTAA
TTAAGACTGTATGGAAAATGAGAGC 3′;
Donor DELTAJDC2:
(SEQ ID NO: 93)
5′CCCCAAGATGACTATCTTTAATGTCTGGAACGATCATCAG
AATTGATACTGACTGTATGGAAAATG 3′;
and
Donor DELTA32RSB:
(SEQ ID NO: 94)
5′GATGACTATCTTTAATGTCTGGAAATTCTACTAGAATTGA
TACTGACTGTATGGAAAATGAGAGC 3’,

or a functional fragment of SEQ ID NO:92, 93, or 94 that is suitable and sufficient to introduce mutation CCR5 gene.

6. Lysosomal Storage Diseases

The compositions and methods can also be used to treat lysosomal storage diseases. Lysosomal storage diseases (LSDs) are a group of more than 50 clinically-recognized, rare inherited metabolic disorders that result from defects in lysosomal function (Walkley, J. Inherit. Metab. Dis., 32(2):181-9 (2009)). Lysosomal storage disorders are caused by dysfunction of the cell's lysosome orangelle, which is part of the larger endosomal/lysosomal system. Together with the ubiquitin-proteosomal and autophagosomal systems, the lysosome is essential to substrate degradation and recycling, homeostatic control, and signaling within the cell. Lysosomal dysfunction is usually the result of a deficiency of a single enzyme necessary for the metabolism of lipids, glycoproteins (sugar containing proteins) or mucopolysaccharides (long unbranched polysaccharides consisting of a repeating disaccharide unit; also known as glycosaminoglycans, or GAGs) which are fated for breakdown or recycling. Enzyme deficiency reduces or prevents break down or recycling of the unwanted lipids, glycoproteins, and GAGs, and results in buildup or “storage” of these materials within the cell. Most lysosomal diseases show widespread tissue and organ involvement, with brain, viscera, bone and connective tissues often being affected. More than two-thirds of lysosomal diseases affect the brain. Neurons appear particularly vulnerable to lysosomal dysfunction, exhibiting a range of defects from specific axonal and dendritic abnormalities to neuron death.

Individually, LSDs occur with incidences of less than 1:100,000, however, as a group the incidence is as high as 1 in 1,500 to 7,000 live births (Staretz-Chacham, et al., Pediatrics, 123(4):1191-207 (2009)). LSDs are typically the result of inborn genetic errors. Most of these disorders are autosomal recessively inherited, however a few are X-linked recessively inherited, such as Fabry disease and Hunter syndrome (MPS II). Affected individuals generally appear normal at birth, however the diseases are progressive. Develop of clinical disease may not occur until years or decades later, but is typically fatal. Lysosomal storage diseases affect mostly children and they often die at a young and unpredictable age, many within a few months or years of birth. This makes these types of lysosomal storage diseases attractive for pre-natal intervention. Many other children die of this disease following years of suffering from various symptoms of their particular disorder. Clinical disease may be manifest as mental retardation and/or dementia, sensory loss including blindness or deafness, motor system dysfunction, seizures, sleep and behavioral disturbances, and so forth. Some people with Lysosomal storage disease have enlarged livers (hepatomegaly) and enlarged spleens (splenomegaly), pulmonary and cardiac problems, and bones that grow abnormally.

Treatment for many LSDs is enzyme replacement therapy (ERT) and/or substrate reduction therapy (SRT), as wells as treatment or management of symptoms. The average annual cost of ERT in the United

States ranges from 90,000 to 565,000. While ERT has significant systemic clinical efficacy for a variety of LSDs, little or no effects are seen on central nervous system (CNS) disease symptoms, because the recombinant proteins cannot penetrate the blood-brain barrier. Allogeneic hematopoietic stem cell transplantation (HSCT) represents a highly effective treatment for selected LSDs. It is currently the only means to prevent the progression of associated neurologic sequelae. However, HSCT is expensive, requires an HLA-matched donor and is associated with significant morbidity and mortality. Recent gene therapy studies suggest that LSDs are good targets for this type of treatment.

Compositions and methods for targeted gene therapy using triplex-forming oligonucleotides and peptide nucleic acids for treating lysosomal storage diseases are described in WO 2011/133802, which provides sequences of triplex forming molecules, target sequences, and donor oligonucleotides that can be utilized in the compositions and methods provided herein.

For example, the compositions and methods can be are employed to treat Gaucher's disease (GD). Gaucher's disease, also known as Gaucher syndrome, is the most common lysosomal storage disease. Gaucher's disease is an inherited genetic disease in which lipid accumulates in cells and certain organs due to deficiency of the enzyme glucocerebrosidase (also known as acid β-glucosidase) in lysosomes. Glucocerebrosidase enzyme contributes to the degradation of the fatty substance glucocerebroside (also known as glucosylceramide) by cleaving b-glycoside into b-glucose and ceramide residues (Scriver C R, Beaudet A L, Valle D, Sly W S. The metabolic and molecular basis of inherited disease. 8th ed. New York: McGraw-Hill Pub, 2001: 3635-3668). When the enzyme is defective, the substance accumulates, particularly in cells of the mononuclear cell lineage, and organs and tissues including the spleen, liver, kidneys, lungs, brain and bone marrow.

There are two major forms: non-neuropathic (type 1, most commonly observed type in adulthood) and neuropathic (type 2 and 3). GBA (GBA glucosidase, beta, acid), the only known human gene responsible for glucosidase-mediated GD, is located on chromosome 1, location 1q21. More than 200 mutations have been defined within the known genomic sequence of this single gene (NCBI Reference Sequence: NG_009783.1). The most commonly observed mutations are N370S, L444P, RecNcil, 84GG, R463C, recTL and 84 GG is a null mutation in which there is no capacity to synthesize enzyme. However, N370S mutation is almost always related with type 1 disease and milder forms of disease. Very rarely, deficiency of sphingolipid activator protein (Gaucher factor, SAP-2, saposin C) may result in GD. In some embodiments, triplex-forming molecules are used to induce recombination of donor oligonucleotides designed to correct mutations in GBA.

In another embodiment, compositions and the methods herein are used to treat Fabry disease (also known as Fabry's disease, Anderson-Fabry disease, angiokeratoma corporis diffusum and alpha-galactosidase A deficiency), a rare X-linked recessive disordered, resulting from a deficiency of the enzyme alpha galactosidase A (a-GAL A, encoded by GLA). The human gene encoding GLA has a known genomic sequence (NCBI Reference Sequence: NG_007119.1) and is located at Xp22 of the X chromosome. Mutations in GLA result in accumulation of the glycolipid globotriaosylceramide (abbreviated as Gb3, GL-3, or ceramide trihexoside) within the blood vessels, other tissues, and organs, resulting in impairment of their proper function (Karen, et al., Dermatol. Online J., 11 (4): 8 (2005)). The condition affects hemizygous males (i.e. all males), as well as homozygous, and potentially heterozygous (carrier), females. Males typically experience severe symptoms, while women can range from being asymptomatic to having severe symptoms. This variability is thought to be due to X-inactivation patterns during embryonic development of the female. In some embodiments, triplex-forming molecules are used to induce recombination of donor oligonucleotides designed to correct mutations in GLA.

In preferred embodiments, the compositions and methods are used to treat Hurler syndrome (HS). Hurler syndrome, also known as mucopolysaccharidosis type I (MPS I), α-L-iduronidase deficiency, and Hurler's disease, is a genetic disorder that results in the buildup of mucopolysaccharides due to a deficiency of α-Liduronidase, an enzyme responsible for the degradation of mucopolysaccharides in lysosomes (Dib and Pastories, Genet. Mol. Res., 6(3):667-74 (2007)). MPS I is divided into three subtypes based on severity of symptoms. All three types result from an absence of, or insufficient levels of, the enzyme a-L-iduronidase. MPS I H or Hurler syndrome is the most severe of the MPS I subtypes. The other two types are MPS I S or Scheie syndrome and MPS I H-S or Hurler-Scheie syndrome. Without a-L-iduronidase, heparan sulfate and dermatan sulfate, the main components of connective tissues, build-up in the body. Excessive amounts of glycosaminoglycans (GAGs) pass into the blood circulation and are stored throughout the body, with some excreted in the urine. Symptoms appear during childhood, and can include developmental delay as early as the first year of age. Patients usually reach a plateau in their development between the ages of two and four years, followed by progressive mental decline and loss of physical skills (Scott et al., Hum. Mutat. 6: 288-302 (1995)). Language may be limited due to hearing loss and an enlarged tongue, and eventually site impairment can result from clouding of cornea and retinal degeneration. Carpal tunnel syndrome (or similar compression of nerves elsewhere in the body) and restricted joint movement are also common.

a. Exemplary Target Sites

The human gene encoding alpha-L-iduronidase (α-L-iduronidase; IDUA) is found on chromosome 4, location 4p16.3, and has a known genomic sequence (NCBI Reference Sequence: NG_008103.1). Two of the most common mutations in IDUA contributing to Hurler syndrome are the Q70X and the W420X, non-sense point mutations found in exon 2 (nucleotide 774 of genomic DNA relative to first nucleotide of start codon) and exon 9 (nucleotide 15663 of genomic DNA relative to first nucleotide of start codon) of IDUA respectively. These mutations cause dysfunction alpha-L-iduronidase enzyme. Two triplex-forming molecule target sequences including a polypurine:polypyrimidine stretches have been identified within the IDUA gene. One target site with the polypurine sequence 5′ CTGCTCGGAAGA 3′ (SEQ ID NO:87) and the complementary polypyrimidine sequence 5′ TCTTCCGAGCAG 3′ (SEQ ID NO:98) is located 170 base pairs downstream of the Q70X mutation. A second target site with the polypurine sequence 5′ CCTTCACCAAGGGGA 3′ (SEQ ID NO:88) and the complementary polypyrimidine sequence 5′ TCCCCTTGGTGAAGG 3′ (SEQ ID NO:95) is located 100 base pairs upstream of the W402X mutation. In preferred embodiments, triplex-forming molecules are designed to bind/hybridize in or near these target locations.

b. Exemplary Triplex Forming Sequences and Donors

i. W402X mutation

In some embodiments, a triplex-forming molecule is a peptide nucleic acid oligomer that binds to the target sequence upstream of the W402X mutation and include the nucleic acid sequence TTCCCCT, preferably includes the sequence TTCCCCT linked to the sequence TCCCCTT, or more preferably includes the sequence TTCCCCT linked to the sequence TCCCCTTGGTGAAGG (SEQ ID NO:95),

and one or more of the peptide nucleic acid residues is a ^{ser}γPNA residue.

In some preferred embodiments, the triplex forming nucleic acid is a peptide nucleic acid oligomer that binds to the target sequence upstream of the W402X mutation including the sequence TTJJJJT, linked to the sequence TCCCCTT or TCCCCTTGGTGAAGG (SEQ ID NO:95),

wherein one or more of the PNA residues is a γPNA.

In specific embodiments, the triplex forming nucleic acid is a peptide nucleic acid oligomer having the sequence Lys-Lys-Lys-TTJJJJT-OOO-TCCCCTTGGTGAAGG-Lys-Lys-Lys (SEQ ID NO:96) (IDUA402tc715) optionally, but preferably wherein one or more of the PNA residues is a ^{ser}γPNA. In even more specific embodiments, the bolded and underlined residues are ^{ser}γPNA residues.

In the most preferred embodiments, triplex-forming molecules are administered according to the methods in combination with one or more donor oligonucleotides designed to correct the point mutations at Q70X or W402X mutations sites. In some embodiments, in addition to containing sequence designed to correct the point mutation at Q70X or W402X mutation, the donor oligonuclotides may also contain 7 to 10 additional, synonymous (silent) mutations. The additional silent mutations can facilitate detection of the corrected target sequence using allele-specific PCR of genomic DNA isolated from treated cells.

In some embodiments, the donor oligonucleotide with the sequence 5′ AGGACGGTCCCGGCCTGCGACACTTCCGCCCATAATTGTTCTTCAT

CTGCGGGGCGGGGGGGGG 3′ (SEQ ID NO:97), or a functional fragment thereof that is suitable and sufficient to correct the W402X mutation is administered with triplex-forming molecules designed to target the binding site upstream of W402X to correct the W402X mutation in cells.

ii. Q70X Mutation

In some embodiments, a triplex-forming molecule is a peptide nucleic acid oligomer that binds to the target sequence downstream of the Q70X mutation and includes the nucleic acid sequence CCTTCT, preferably includes the sequence CCTTCT linked to the sequence TCTTCC, or more preferably includes the sequence CCTTCT linked to the sequence TCTTCCGAGCAG (SEQ ID NO:98),

and one or more of the peptide nucleic acid residues is a serγPNA residue.

In preferred embodiments, the triplex forming nucleic acid is a peptide nucleic acid oligomer that binds to the target sequence downstream of the Q70X mutation including the sequence JJTTJT linked to the sequence TCTTCC or TCTTCCGAGCAG (SEQ ID NO:98);

wherein one or more of the PNA residues is a γPNA.

In a specific embodiment, a tcPNA with a sequence of Lys-Lys-Lys-JJTTJT-OOO-TCTTCCGAGCAG-Lys-Lys-Lys (SEQ ID NO:99) (IDUA402tc715), wherein one or more of the PNA residues is a ^{ser}γPNA. In even more specific embodiments, the bolded and underlined residues ^{ser}γPNA residues.

A donor oligonucleotide can have the sequence 5′GGGACGGCGCCCACATAGGCCAAATTCAATTGCTGATCCCAGCT TAAGACGTACTGGTCAGCCTGGC 3′ (SEQ ID NO:83), or a functional fragment thereof that is suitable and sufficient to correct the Q70X mutation is administered with triplex-forming molecules designed to target the binding site downstream of Q70X to correct the of Q70X mutation in cells.

### IV. Combination Therapies

Each of the different active agents including components of gene editing and potentiation here can be administered alone or in any combination and further in combination with one or more additional active agents. In all cases, the combination of agents can be part of the same admixture, or administered as separate compositions. In some embodiments, the separate compositions are administered through the same route of administration. In other embodiments, the separate compositions are administered through different routes of administration.

A. Conventional Therapeutic Agents

Examples of preferred additional active agents include other conventional therapies known in the art for treating the desired disease or condition. For example, in the treatment of sickle cell disease, the additional therapy may be hydroxurea.

In the treatment of cystic fibrosis, the additional therapy may include mucolytics, antibiotics, nutritional agents, etc. Specific drugs are outlined in the Cystic Fibrosis Foundation drug pipeline and include, but are not limited to, CFTR modulators such as KALYDECO® (invascaftor), ORKAMBI™ (lumacaftor +ivacaftor), ataluren (PTC124), VX-661+invacaftor, riociguat, QBW251, N91115, and QR-010; agents that improve airway surface liquid such as hypertonic saline, bronchitol, and P-1037; mucus alteration agents such as PULMOZYME® (dornase alfa); anti-inflammatories such as ibuprofen, alpha 1 anti-trypsin, CTX-4430, and JBT-101; anti-infective such as inhaled tobramycin, azithromycin, CAYSTON® (aztreonam for inhalation solution), TOBI inhaled powder, levofloxacin, ARIKACE® (nebulized liposomal amikacin), AEROVANC® (vancomycin hydrochloride inhalation powder), and gallium; and nutritional supplements such as aquADEKs, pancrelipase enzyme products, liprotamase, and burlulipase.

In the treatment of HIV, the additional therapy maybe an antiretroviral agents including, but not limited to, a non-nucleoside reverse transcriptase inhibitor (NNRTIs), a nucleoside reverse transcriptase inhibitor (NRTIs), a protease inhibitors (PIs), a fusion inhibitors, a CCR5 antagonists (CCR5s) (also called entry inhibitors), an integrase strand transfer inhibitors (INSTIs), or a combination thereof.

In the treatment of lysosomal storage disease, the additional therapy could include, for example, enzyme replacement therapy, bone marrow transplantation, or a combination thereof.

B. Additional Mutagenic Agents

The compositions can be used in combination with other mutagenic agents. In a preferred embodiment, the additional mutagenic agents are conjugated or linked to gene editing technology or a delivery vehicle (such as a nanoparticle or microparticle) thereof. Additional mutagenic agents that can be used in combination with gene editing technology, particularly triplex forming molecules, include agents that are capable of directing mutagenesis, nucleic acid crosslinkers, radioactive agents, or alkylating groups, or molecules that can recruit DNA-damaging cellular enzymes. Other suitable mutagenic agents include, but are not limited to, chemical mutagenic agents such as alkylating, bialkylating or intercalating agents. A preferred agent for co-administration is psoralen-linked molecules as described in PCT/US/94/07234 by Yale University.

It may also be desirable to administer gene editing compositions in combination with agents that further enhance the frequency of gene modification in cells. For example, the compositions can be administered in combination with a histone deacetylase (HDAC) inhibitor, such as suberoylanilide hydroxamic acid (SAHA), which has been found to promote increased levels of gene targeting in asynchronous cells.

The nucleotide excision repair pathway is also known to facilitate triplex-forming molecule-mediated recombination. Therefore, the compositions can be administered in combination with an agent that enhances or increases the nucleotide excision repair pathway, for example an agent that increases the expression, or activity, or localization to the target site, of the endogenous damage recognition factor XPA.

Compositions may also be administered in combination with a second active agent that enhances uptake or delivery of the gene editing technology. For example, the lysosomotropic agent chloroquine has been shown to enhance delivery of PNAs into cells (Abes, et al., J. Controll. Rel., 110:595-604 (2006). Agents that improve the frequency of gene modification are particularly useful for in vitro and ex vivo application, for example ex vivo modification of hematopoietic stem cells for therapeutic use.

### V. Methods for Determining Triplex Formation and Gene Modification

A. Methods for Determining Triplex Formation

A useful measure of triple helix formation is the equilibrium dissociation constant, K_{d}, of the triplex, which can be estimated as the concentration of triplex-forming molecules at which triplex formation is half-maximal. Preferably, the molecules have a binding affinity for the target sequence in the range of physiologic interactions. Preferred triplex-forming molecules have a K_{d} less than or equal to approximately 10⁻⁷ M. Most preferably, the K_{d} is less than or equal to 2×10⁻⁸ M in order to achieve significant intramolecular interactions. A variety of methods are available to determine the K_{d} of triplex-forming molecules with the target duplex. In the examples which follow, the K_{d} was estimated using a gel mobility shift assay (R. H. Durland et al., Biochemistry 30, 9246 (1991)). The dissociation constant (K_{d}) can be determined as the concentration of triplex-forming molecules in which half was bound to the target sequence and half was unbound.

B. Methods for Determining Gene Modification

Sequencing and allele-specific PCR are preferred methods for determining if gene modification has occurred. PCR primers are designed to distinguish between the original allele, and the new predicted sequence following recombination. Other methods of determining if a recombination event has occurred are known in the art and may be selected based on the type of modification made. Methods include, but are not limited to, analysis of genomic DNA, for example by sequencing, allele-specific PCR, or restriction endonuclease selective PCR (REMS-PCR); analysis of mRNA transcribed from the target gene for example by Northern blot, in situ hybridization, real-time or quantitative reverse transcriptase (RT) PCT; and analysis of the polypeptide encoded by the target gene, for example, by immunostaining, ELISA, or FACS. In some cases, modified cells will be compared to parental controls. Other methods may include testing for changes in the function of the RNA transcribed by, or the polypeptide encoded by the target gene. For example, if the target gene encodes an enzyme, an assay designed to test enzyme function may be used.

### VI. Kits

Medical kits are also disclosed. The medical kits can include, for example, a dosage supply of gene editing technology or a potentiating agent thereof, or a combination thereof in separately or together in the same admixture. The active agents can be supplied alone (e.g., lyophilized), or in a pharmaceutical composition. The active agents can be in a unit dosage, or in a stock that should be diluted prior to administration. In some embodiments, the kit includes a supply of pharmaceutically acceptable carrier. The kit can also include devices for administration of the active agents or compositions, for example, syringes. The kits can include printed instructions for administering the compound in a use as described above.

The disclosed compositions and methods can be further understood through the following numbered paragraphs.

1. A peptide nucleic acid oligomer comprising a Hoogsteen binding peptide nucleic acid (PNA) segment and a Watson-Crick binding PNA segment collectively totaling no more than 50 PNA residues in length, wherein the two segments can bind or hybridize to a target region comprising a polypurine stretch in a cell's genome to induce strand invasion, displacement, and formation of a triple-stranded molecule among the two PNA segments and the polypurine stretch of the cell's genome, wherein the Hoogsteen binding segment binds to the target duplex by Hoogsteen binding for a length of least five nucleobases, wherein the Watson-Crick binding segment binds to the target duplex by Watson-Crick binding for a length of least five nucleobases, and wherein one or more (e.g., any integer number between 1 and 50, inclusive) of the PNA residues have a hydroxymethyl-modification at the gamma position (“^{Ser}γPNA”), optionally, wherein alternating residues in the Hoogsteen binding segment, Watson-Crick binding segment, or combination thereof have a hydroxymethyl-modification at the gamma position (“^{ser}γPNA”) and are unmodified at the gamma position, respectively.

2. The peptide nucleic acid oligomer of paragraph 1, wherein the Hoogsteen binding segment comprises one or more chemically modified cytosines selected from the group consisting of pseudocytosine, pseudoisocytosine, and 5-methylcytosine.

3. The peptide nucleic acid oligomer of paragraphs 1 or 2, wherein the Watson-Crick binding segment comprises a tail sequence of up to fifteen nucleobases that binds to the target duplex by Watson-Crick binding outside of the triplex.

4. The peptide nucleic acid oligomer of any one of paragraphs 1-3 wherein the two segments are linked by a linker.

5. The peptide nucleic acid oligomer of paragraph 4, wherein the linker is between 1 and 10 units of 8-amino-3,6-dioxaoctanoic acid, 6-aminohexanoic acid, 8-amino-2, 6, 10-trioxaoctanoic acid, or 11-amino-3,6,9-trioxaundecanoic acid.

6. The peptide nucleic acid oligomer of any one of paragraphs 1-5, wherein all of the peptide nucleic acid residues in the Hoogsteen binding segment only, in the Watson-Crick binding segment only, or across the entire PNA oligomer are ^{ser}γPNA residues. 7. The peptide nucleic acid oligomer of any one of paragraphs 1-5, wherein one or more of the peptide nucleic acid residues in the Hoogsteen binding segment only or in the Watson-Crick binding segment only of the PNA oligomer or across the entire PNA oligomer are ^{ser}γPNA residues.

8. The peptide nucleic acid oligomer of any one of paragraphs 1-5, wherein alternating residues in the Hoogsteen binding portion only, in the Watson-Crick binding portion only, or across the entire PNA oligomer are PNA and ^{ser}γPNA.

9. The peptide nucleic acid oligomer of any one of paragraphs 1-8, wherein one or more of the cytosines is replaced with a G-clamp (9-(2-guanidinoethoxy) phenoxazine).

10. The peptide nucleic acid oligomer of any one of paragraphs 1-9 wherein the N-terminus, the C-terminus, or both comprise 1, 2, 3 or more lysines.

11. A pharmaceutical composition comprising an effective amount of the peptide nucleic acid oligomer of any one of paragraphs 1-10.

12. The pharmaceutical composition of paragraph 11 further comprising a donor oligonucleotide comprising a sequence that can correct a mutation(s) in a cell's genome by recombination induced or enhanced by the peptide nucleic acid oligomer.

13. The pharmaceutical composition of paragraphs 11 or 12 further comprising nanoparticles, wherein the PNA oligomer, donor oligonucleotide, or a combination thereof are packaged in the same or separate nanoparticles.

14. The pharmaceutical composition of paragraph 13, wherein the nanoparticle comprises poly(lactic-co-glycolic acid) (PLGA).

15. The pharmaceutical composition of paragraphs 13 or 14, wherein the nanoparticles comprise poly(beta-amino) esters (PBAEs).

16. The pharmaceutical composition of paragraph 15, wherein the nanoparticles comprise a blend of PLGA and PBAE comprising about between about 5 and about 25 percent PBAE (wt %).

17. The pharmaceutical composition of any one of paragraphs 11-16 further comprising a targeting moiety, a cell penetrating peptide, or a combination thereof associated with, linked, conjugated, or otherwise attached directly or indirectly to the PNA oligomer or the nanoparticles.

18. A method of modifying the genome of a cell comprising contacting the cell with the pharmaceutical composition of any one of paragraphs 11-17.

19. The method of paragraph 18 wherein the contacting occurs in vitro, ex vivo, or in vivo.

20. The method of paragraph 19, wherein the contacting occurs in vivo, the subject has a genetic disease or disorder caused by a genetic mutation, and the pharmaceutical composition is administered to the subject in an effective amount to correct the mutation in an effective number of cells to reduce one or more symptoms of the disease or disorder.

21. The method of paragraph 20 further comprising administering to the subject an effective amount of a potentiating agent to increase the frequency of recombination of the donor oligonucleotide at a target site in the genome of a population of cells.

22. The method of any one of paragraphs 18-21, wherein the peptide nucleic acid oligomer can induce a higher frequency of recombination in a population of target cells as a corresponding peptide nucleic acid oligomer wherein the ^{ser}γPNA residues are replaced with mini-PEGγPNA residues or are unmodified.

23. The method of any one of paragraphs 20-22 wherein the genetic disease or disorder is selected from the group consisting of cystic fibrosis, hemophilia, globinopathies, xeroderma pigmentosum, lysosomal storage diseases, HIV, or cancer.

24. The method of paragraph 23, wherein the genetics disease or disorder is a globinopathy selected from sickle cell anemia and beta-thalassemia.

25. The method of paragraph 23, wherein the genetic disease or disorder is cystic fibrosis.

26. The peptide nucleic acid, or pharmaceutical composition or method of use thereof, of any of the preceding paragraphs comprising a peptide nucleic acid sequence disclosed herein.

27. The peptide nucleic acid oligomer of any of the preceding paragraphs comprising the sequence JTTTJTTTJTJT-OOO-TCTCTTTCTTTCAGGGCA (SEQ ID NO:16), wherein the ^{ser}γPNA residues are underlined, unmodified residues have no underlining, “J” is pseudoisocytosine, and each “O” is an 8-amino-3,6-dioxaoctanoic acid, 6-aminohexanoic acid, 8-amino-2, 6, 10-trioxaoctanoic acid, or 11-amino-3,6,9-trioxaundecanoic acid moiety.

The present invention will be further understood by reference to the following non-limiting examples.

### EXAMPLES

### Example 1

### PNA Oligomer Synthesis, Isolation, and Characterization.

### Materials and Methods

PNA Synthesis, Purification, and Characterization

PNA oligomers possessing the gamma (γ)-hydroxymethyl substitution (^{ser}γPNA) were synthesized using established protocols for standard solid phase peptide syntheses that have been previously adapted for PNA synthesis (Manna, et al., Methods in Molecular Biology, 1050:1-12 (2014). doi: 10.1007/978-1-62703-553-8_1). All hydroxymethyl PNA monomers were purchased as boc-protected amino acids from ASM Research Chemicals GmbH (Hannover, Germany) and used directly for synthesis. Oligomer synthesis was initiated on an MBHA solid support (resin) labelled in-house with a lysine residue. The entire synthesis consisted of iterative cycles of two major steps: (1) amine deprotection with a solution of trifluoroacetic acid/m-cresol (95/5); and (2) monomer coupling with a cocktail of monomer (A,G,T,or C), DIEA, and HBTU. After completion of the cycle for the last monomer, the oligomer was cleaved (released) by submerging the resin in a solution of m-cresol/thioanisole/trifluoroacetic acid/trifluroromethane sulfonic acid (1/1/2/6). The pure product was isolated by reverse phase (RP)-HPLC, using a solvent gradient of acetonitrile and water, and validated using MALDI mass spectrometry.

### Results

Iterative cycles of amine deprotection and monomer coupling were used for continuous oligomer extension to obtain ^{ser}γPNA. Once released from the resin, a crude solid was isolated by precipitation in diethyl ether, followed by isolation of the pure oligomer by RP-HPLC. Confirmatory analyses, including mass spectrometry and chromatography, were used to verify the identity and purity, respectively, of the synthesized compound.

In brief, the chromatogram for the isolated product shows a single major peak adjacent to a very minor shoulder. Fractional abundance measurements on both peaks show that the major component (^{ser}γPNA) accounts for 98% of the total isolated mixture. Further, the expected mass for ^{ser}γPNA (9198 g/mol) was observed in the MALDI spectrum of this isolated product, even though lower masses—likely attributable to truncations arising during synthesis or spectrometry itself—are present. This level of purity and validation was determined to be sufficient for subsequent experiments, which themselves further confirmed the compound identity as well as its utility for gene editing applications (see below).

The nucleobase sequence for ^{ser}γPNA, along with those for isosequential PNA/γPNA oligomers used as control or comparative samples, are provided below in Table 1.

TABLE 1
Sequences of ^{ser}γPNA, ^{mp}γPNA, and PNA oligomers.

| Oligomer | Sequence |
|---|---|
| ^{ser}γPNA | JTTTJTTTJTJT-OOO- |
| | TCTCTTTCTTTCAGGGCA (SEQ ID |
| | NO: 16), wherein the ^{ser}γPNA |
| | residues are underlined. |
| ^{mp}γPNA (γtcPNA4) | JTTTJTTTJTJT-OOO- |
| (Bahal, Nature | TCTCTTTCTTTCAGGGCA (SEQ ID |
| Communications. | NO: 16), wherein the ^{mp}γPNA |
| 2016; 7: 13304. doi: | residues are underlined. |
| 10.1038/ncomms13304. | |
| PubMed PMID: | |
| 27782131; PMCID: | |
| PMC5095181) | |
| PNA (no gamma | JTTTJTTTJTJT-OOO- |
| modification) | TCTCTTTCTTTCAGGGCA (SEQ ID |
| | NO: 16) |

All γPNA/PNAs in Table 1 are presented from N- to C-termini, and have three consecutive lysine residues on each terminus. J=pseudoisocytidine, a cytosine isomer that mimics the protonated form of cytosine, and can thus form Hoogsteen H-bonds under neutral pH conditions; OOO=an 11-Amino-3,6,9-trioxaundecanoic acid linker between the tail and clamp PNA domains, which, respectively, bind to the Watson-Crick and Hoogsteen faces of the target DNA. Underlined sequences denote positions of γ-modification.

### Example 2

### ^{ser}γPNA Exhibit Helical Preorganization

### Materials and Methods

Circular Dichroism (CD) Spectropolarimetry

Equimolar amounts (5 μM) of each of the respective PNA/γPNA samples were prepared in a buffer containing 10 mM Na₃PO₄ (NaPi, pH 7.4). A ‘blank’ sample containing the buffer alone was used as a negative control. All samples were annealed by heating (95° C., 5 min) and slow cooling on a heat block to allow for formation of the most stable conformations or secondary structures within/by each PNA/γPNA oligomer. CD spectra were recorded on a Chirascan CD spectropolarimeter (Applied Photophysics). All spectra were collected from 200-350 nm, baseline corrected, and recorded as the average of three consecutive scans.

### Result

γ-substitutions in PNA monomers have been reported to confer, among other properties, helical pre-organization in the corresponding oligomers (Dragulescu-Andrasi, et al., Journal of the American Chemical Society, 128(31):10258-67 (2006)). CD experiments to evaluate the helicity of ^{ser}γPNA relative to an isosequential PNA compound (γtcPNA4, referred to here as ^{mp}γPNA) possessing the ethylene glycol substitution at the same position of the PNA backbone (formula above), and with the same periodicity on the oligomer (Table 1). The CD spectrum of ^{ser}γPNA displays characteristic signatures of right-handed helical PNA oligomers (Dragulescu-Andrasi, et al., Journal of the American Chemical Society, 128(31):10258-67 (2006). doi: 10.1021/j a0625576; Sahu, et al., Journal of Organic Chemistry, 76(14):5614-27 (2011) doi: 10.1021/jo200482d), such as local minimum and maximum at 240- and 267 nm, respectively (FIG. 1). Interestingly, ^{ser}γPNA appears more strongly preorganized than ^{mp}γPNA, whose diagnostic CD signatures, although occurring at identical positions, present at weaker intensities (FIG. 1).

### Example 3

### ^{ser}γPNA Binds DNA

### Materials and Methods

Electrophoretic Mobility (Gel) Shift Assays (EMSA) Complementary 60-nucleotide synthetic DNA oligomers containing the PNAs' binding site were prepared in a 10 mM NaPi buffer (pH 7.4) and annealed by heating and subsequent slow cooling (95-37° C.) on a heat block to form the target duplex. The respective γPNAs were then mixed with the pre-formed DNA duplex target in 10 mM NaPi buffer (pH 7.4), and the resulting solutions were either incubated (24 h) at 37° C., or annealed (1 h) on a heating block. The PNA-bound and intact DNA fragments were separated electrophoretically on a nondenaturing 8% PAGE gel by running at 120 V for 45 min in 1× tris-borate EDTA buffer. The resolved complexes were visualized by staining the gel with lx SYBR gold nucleic acid gel stain (Invitrogen, #S11494), washing the gel for 10 min in 1× TBE, and imaging on a gel documentation system (ChemiDoc™ XRS+, Biorad).

Surface Plasmon Resonance (SPR)

All experiments were run on a Biacore T100 system (GE Healthcare Life Sciences) within the Biophysical core of the Keck Foundation Biotechnology Resource Laboratory at Yale University. In preparation for the experiments, synthetic 5′-biotinylated DNA oligos, purchased from Integrated DNA Technologies, were deposited on designated channels of a four-channel SA Series S sensor chip (GE Healthcare Life Sciences) according to published protocols (Armitage, et al., Methods Mol Biol. 1050:159-65 (2014). doi: 10.1007/978-1-62703-553-8_13. PubMed PMID: 24297358.). The SA sensor chips are functionalized by the commercial vendor with recombinant streptavidin, and allow for high-capacity immobilization of biotinylated analytes by exploiting the specific, high-affinity biotin/streptavidin non-covalent interaction. For hybridization experiments, a low density (150 response units, RU) of the target DNA oligomer was immobilized on the chip by injecting a 200 nM solution of the DNA oligomer in immobilization buffer [100 mM NaPi, pH 7.4, 0.002% surfactant P-20 (GE Healthcare Life Sciences)]. For all experiments, a reference channel devoid of any immobilized substrate was used to isolate and subtract non-specific ligand-surface interactions from specific probe binding. Each hybridization experiment was performed by injecting a 10 nM solution of the respective PNA/γPNAs [dissolved in running buffer (10/100 mM NaPi, pH 7.4, 0.002% P-20)] over both the active and reference flow cells. The binding sensorgrams for all analytes were recorded as the differentials of the binding responses between the active and reference channels. A sensorgram for the buffer was also collected (by running a blank sample), and used to correct for any bulk refractive index effects in the sample sensorgrams.

### Results

The interaction of ^{ser}γPNA with its cognate DNA target sequence was evaluated using two assays: EMSA and SPR experiments. For the former, two parameters were tested: annealing—short heating, slow cooling—of the PNA oligomer mixed with a 60-mer DNA duplex containing the PNA binding site in 10 mM NaPi (pH 7.4) buffer; and incubation (at 37° C., for 24 h) of the two binding components in the same buffer. The emergence of a significantly retarded band, corresponding to the PNA/DNA complex, was observed in the presence of the ^{ser}γPNA under both annealing (95-37° C.) and incubation (37° C.) conditions. Importantly, the results indicate that the interaction of ^{ser}γPNA with the DNA target is similar to the corresponding interaction involving the isosequential mPγPNA, even though the latter compound shows, qualitatively, greater (if marginal) complex formation with the target DNA under conditions of incubation.

SPR experiments (Sahu, et al., Journal of Organic Chemistry, 76(14):5614-27 (2011). doi: 10.1021/jo200482d. PubMed PMID: 21619025; PMCID: PMC3175361) were utilized for a more quantitative evaluation of the PNA/γPNA-DNA interactions. An 80mer DNA oligomer containing the PNA binding site was immobilized on an SPR chip, which was in turn exposed to a flowing solution of the respective PNA oligomer. PNA exposure (the association phase) is continued at a constant flow rate for 420 s, and is followed by buffer exposure (dissociation phase) for 600 s. Significantly higher DNA was observed binding when ^{ser}γPNA is injected over the chip (FIG. 2) than for the respective exposures to the isosequential oligomers possessing either ethylene glycol γ-substitutions (^{mp}γ, FIG. 2) or none at all (^{no}γ, FIG. 2). Further, the initial association rate for ^{ser}γPNA—defined here as the time required for a response change of 30 units, 10 s after the injection start—is 2.5- and 3-fold greater than those for ^{mp}γPNA and the unmodified (^{no}γ) PNA, respectively (FIG. 2).

### Example 4

### ^{ser}γPNA Can Edit Bone Marrow Cells from β-thalassemia Transgenic Mice

### Materials and Methods

Nanoparticle Synthesis

Polymeric nanoparticles used to deliver gene editing agents were synthesized by a double-emulsion solvent evaporation protocol as described in Bahal, et al., Nature Communications, 7:13304 (2016). doi: 10.1038/ncomms13304. PubMed PMID: 27782131. Briefly, poly(lactic-co-glycolic) acid (PLGA) was dissolved in dichloromethane at a concentration of 40 mg/ml. Prior to encapsulation, PNA and donor DNA were mixed at a 2:1 molar ratio and added dropwise to the PLGA solution under vortex. The resulting mixture was sonicated three times for 10 seconds using an amplitude of 38%. The water-in-oil emulsion was subsequently added dropwise to a surfactant solution containing polyvinyl alcohol (5% w/v). Following the second emulsion, the sonication step was repeated as described. The resulting nanoparticle solution was added to 25 ml of a 0.3% PVA solution and allowed to stir for 3 hours at room temperature. After stirring and particle ‘hardening,’ the nanoparticles were washed 3 times via centrifugation (16,100 g, 15 min, 4° C.) before being flash frozen and lyophilized in cryoprotectant (trehalose, mg:mg). Dry nanoparticles were stored at −20° C. until later use.

Nanoparticle Characterization

Nanoparticles were characterized using the Malvern Zetasizer. Dynamic light scattering (DLS) and zeta potential were obtained to study particle size and surface charge, respectively. Total nucleic acid loading of PNA and donor DNA was measured by optical density (OD) at 260 nm using a NanoDrop (ThermoFisher). Briefly, ˜2 mg of NPs was dissolved in 50 μl of DMSO. Dilutions were made into water prior to measuring absorbance. All values were normalized to total nanoparticle mass (OD/mg/ml).

Ex Vivo Gene Editing

To test the gene editing potential of ser_{y}PNA, a transgenic mouse model of β-thalassemia with a β-globin transgene carrying a thalassemia-associated IVS2-654 (C→T) mutation was used. This splice site mutation leads to expression of abnormal β-globin. Briefly, mice were euthanized using standard protocols approved by the Institutional Animal Care and Use Committees (IACUC). Femurs and tibias were subsequently harvested. and flushed using 1×PBS and strained through a 70 μm filter. Following centrifugation, primary bone marrow cells were seeded in 12 well plates at 500,000 cells per well, using RPMI media supplemented with 1% penicillin-streptomycin (Pen-Strep) and 20% fetal bovine serum (FBS). Cells were treated immediately with 2 mg of nanoparticles containing either ^{ser}γPNA or ^{mp}γPNA along with a correcting donor DNA. After 72 hours, gDNA was extracted from treated primary bone marrow cells using the ReliaPrep gDNA Tissue Miniprep System (Promega). Editing frequencies were subsequently quantified using Digital Droplet PCR (BioRad).

### Results

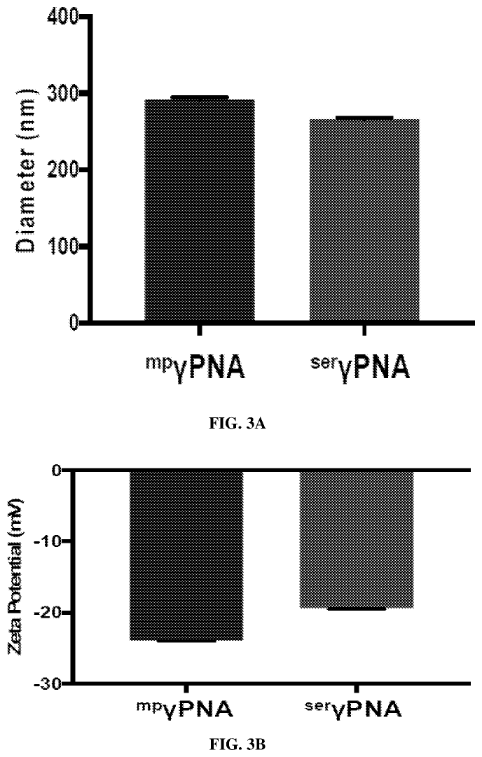

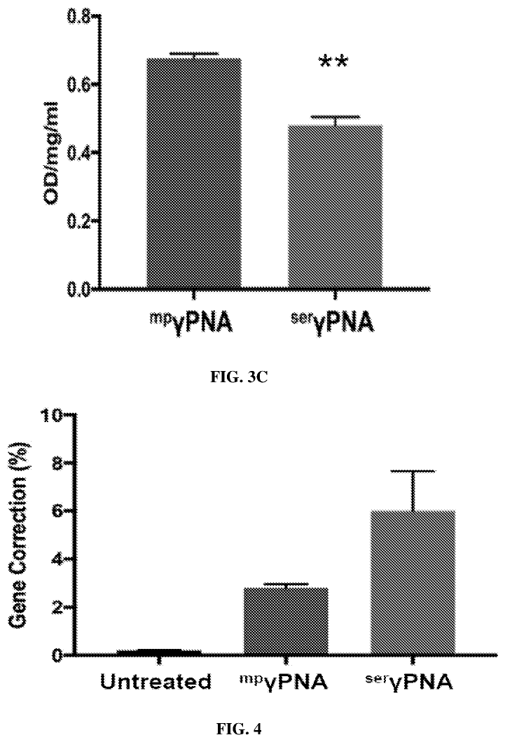

Spherical nanoparticles were synthesized containing ^{mp}γPNA or ^{ser}γPNA and a correcting donor DNA. ^{mp}γPNA NPs were approximately 291.9±2.9 nm (n=3), while ^{ser}γPNA NPs were 266.6±1.5 nm (n=3) (FIG. 3A). Nanoparticle surfaces were negatively charged in both cases (−23.9±0.1 and −19.3±0.2 (n=3) (FIG. 3B)). Loading of total nucleic acids was significantly higher when encapsulating ^{mp}γPNA, with a total OD/mg/ml of 0.68 (n=3) (FIG. 3C).

After nanoparticle synthesis, ^{mp}γPNA and ^{ser}γPNA were tested for their gene editing potential in primary bone marrow cells derived from a mouse model of β-thalassemia. Following 72 h of nanoparticle treatment, gene correction was assessed. Cells treated with nanoparticles containing ^{ser}γPNA and correcting donor DNA resulted in higher levels of gene correction (6.0±1.6% vs. 2.8±0.2%) (n=3) (FIG. 4).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A peptide nucleic acid oligomer comprising a Hoogsteen binding peptide nucleic acid (PNA) segment and a Watson-Crick binding PNA segment collectively totaling no more than 50 PNA residues in length, wherein the two segments can bind or hybridize to a target region comprising a polypurine stretch in a cell's genome to induce strand invasion, displacement, and formation of a triple-stranded molecule among the two PNA segments and the polypurine stretch of the cell's genome,
wherein the Hoogsteen binding segment binds to the target duplex by Hoogsteen binding for a length of least five nucleobases,
wherein the Watson-Crick binding segment binds to the target duplex by Watson-Crick binding for a length of least five nucleobases, and
wherein two or more of the PNA residues have a hydroxymethyl-modification at the gamma position (“^{ser}γPNA”).

2. The peptide nucleic acid oligomer of claim 1, wherein alternating residues in the Hoogsteen binding segment, Watson-Crick binding segment, or a combination thereof have a hydroxymethyl-modification at the gamma position (“^{ser}γPNA”) and are unmodified at the gamma position, respectively.

3. The peptide nucleic acid oligomer of claim 2, wherein alternating residues in the Hoogsteen binding portion only or in the Watson-Crick binding portion only have the hydroxymethyl-modification at the gamma position (“^{ser}γPNA”) and unmodified at the gamma position, respectively.

4. The peptide nucleic acid oligomer of claim 3, wherein all of the peptide nucleic acid residues in the Hoogsteen binding segment only or in the Watson-Crick binding segment only are unmodified at the gamma position.

5. The peptide nucleic acid oligomer of claim 1, wherein the Hoogsteen binding segment comprises one or more chemically modified cytosines selected from the group consisting of pseudocytosine, pseudoisocytosine, and 5-methylcytosine.

6. The peptide nucleic acid oligomer of claim 1, wherein the Watson-Crick binding segment comprises a tail sequence of up to fifteen nucleobases that binds to the target duplex by Watson-Crick binding outside of the triplex.

7. The peptide nucleic acid oligomer of claim 1 wherein the two segments are linked by a linker.

8. The peptide nucleic acid oligomer of claim 7, wherein the linker is between 1 and 10 units of 8-amino-3,6-dioxaoctanoic acid, 6-aminohexanoic acid, 8-amino-2, 6, 10-trioxaoctanoic acid, or 11-amino-3,6,9-trioxaundecanoic acid.

9. The peptide nucleic acid oligomer of claim 1, wherein one or more of the cytosines is replaced with a G-clamp (9-(2-guanidinoethoxy) phenoxazine).

10. The peptide nucleic acid oligomer of claim 1 wherein the N-terminus, the C-terminus, or both comprise 1, 2, 3 or more lysines.

11. The peptide nucleic acid oligomer of claim 1 comprising the sequence JTTTJTTTJTJT-OOO-TCTCTTTCTTCAGGGCA (SEQ ID NO:16), wherein underlined bases are ^{ser}γPNA residues, unmodified residues have no underlining, “J” is pseudoisocytosine, and each “O” is an 8-amino-3,6-dioxaoctanoic acid, 6-aminohexanoic acid, 8-amino-2,6,10-trioxaoctanoic acid, or 11-amino-3,6,9-trioxaundecanoic acid moiety.

12. A pharmaceutical composition comprising an effective amount of the peptide nucleic acid oligomer of claim 1.

13. The pharmaceutical composition of claim 12 further comprising a donor oligonucleotide comprising a sequence that can correct a mutation(s) in a cell's genome by recombination induced or enhanced by the peptide nucleic acid oligomer.

14. The pharmaceutical composition of claim 12 further comprising nanoparticles, wherein the PNA oligomer, donor oligonucleotide, or a combination thereof are packaged in the same or separate nanoparticles.

15. The pharmaceutical composition of claim 14, wherein the nanoparticle comprises poly(lactic-co-glycolic acid) (PLGA).

16. The pharmaceutical composition of claim 14, wherein the nanoparticles comprise poly(beta-amino) esters (PBAEs).

17. The pharmaceutical composition of claim 16, wherein the nanoparticles comprise a blend of PLGA and PBAE comprising about between about 5 and about 25 percent PBAE (wt %).

18. The pharmaceutical composition of claim 12 further comprising a targeting moiety, a cell penetrating peptide, or a combination thereof associated with, linked, conjugated, or otherwise attached directly or indirectly to the PNA oligomer or the nanoparticles.

19. A method of modifying the genome of a cell comprising contacting the cell with the pharmaceutical composition of claim 12.

20. The method of claim 19 wherein the contacting occurs in vitro, ex vivo, or in vivo.

21. The method of claim 20, wherein the contacting occurs in vivo, the subject has a genetic disease or disorder caused by a genetic mutation, and the pharmaceutical composition is administered to the subject in an effective amount to correct the mutation in an effective number of cells to reduce one or more symptoms of the disease or disorder.

22. The method of claim 21 further comprising administering to the subject an effective amount of a potentiating agent to increase the frequency of recombination of the donor oligonucleotide at a target site in the genome of a population of cells.

23. The method of claim 19, wherein the peptide nucleic acid oligomer can induce a higher frequency of recombination in a population of target cells as a corresponding peptide nucleic acid oligomer wherein the ^{ser}γPNA residues are replaced with mini-PEGγ PNA residues or are unmodified.

24. The method of claim 21 wherein the genetic disease or disorder is selected from the group consisting of cystic fibrosis, hemophilia, globinopathies, xeroderma pigmentosum, lysosomal storage diseases, HIV, or cancer.

25. The method of claim 24, wherein the genetics disease or disorder is a globinopathy selected from sickle cell anemia and beta-thalassemia.

26. The method of claim 24, wherein the genetic disease or disorder is cystic fibrosis.

27. (canceled)


SEQUENCE LISTING (99 sequences)

SEQ ID NO: 1 - protein, 11 aa, Artificial Sequence
  Synthetic peptide
YGRKKRRQRRR

SEQ ID NO: 2 - protein, 9 aa, Artificial Sequence
  Synthetic Peptide
RKKRRQRRR

SEQ ID NO: 3 - protein, 16 aa, Artificial Sequence
  Synthetic Peptide
RQIKIWFQNRRMKWKK

SEQ ID NO: 4 - protein, 19 aa, Artificial Sequence
  Synthetic Peptide
HHHHRKKRRQRRRRHHHHH

SEQ ID NO: 5 - protein, 30 aa, Artificial Sequence
  Synthetic Peptide
MVKSKIGSWILVLFVAMWSDVGLCKKRPKP

SEQ ID NO: 6 - protein, 27 aa, Artificial Sequence
  Synthetic Peptide
GALFLGFLGAAGSTMGAWSQPKKKRKV

SEQ ID NO: 7 - DNA, 12 bp, Artificial Sequence
  Synthetic Primer
gaaagaaagaga

SEQ ID NO: 8 - DNA, 18 bp, Artificial Sequence
  Synthetic Primer
tgccctgaaagaaagaga

SEQ ID NO: 9 - DNA, 17 bp, Artificial Sequence
  Synthetic Primer
agaatggtgcaaagagg

SEQ ID NO: 10 - DNA, 18 bp, Artificial Sequence
  Synthetic Primer
acatgattagcaaaaggg

SEQ ID NO: 11 - DNA, 12 bp, Artificial Sequence
  Synthetic Primer
ctttctttctct

SEQ ID NO: 12 - DNA, 12 bp, Artificial Sequence
  Synthetic Primer
tctctttctttc

SEQ ID NO: 13 - DNA, 18 bp, Artificial Sequence
  Synthetic Primer
tctctttctttcagggca

SEQ ID NO: 14 - DNA, 18 bp, Artificial Sequence
  Synthetic Primer
cccttttgctaatcatgt

SEQ ID NO: 15 - DNA, 17 bp, Artificial Sequence
  Synthetic Primer
cctctttgcaccattct

SEQ ID NO: 16 - DNA, 30 bp, Artificial Sequence
  Synthetic Peptide Nucleic Acid
  misc_feature (1)..(1): n = Pseudoisocytosine
  misc_feature (1)..(1): Linked to lys-lys-lys
  misc_feature (5)..(5): n = Pseudoisocytosine
  misc_feature (9)..(9): n = Pseudoisocytosine
  misc_feature (11)..(11): n = Pseudoisocytosine
  misc_feature (12)..(13): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (30)..(30): Linked to lys-lys-lys
ntttntttntnttctctttctttcagggca

SEQ ID NO: 17 - DNA, 25 bp, Artificial Sequence
  Synthetic Peptide Nucleic Acid
  misc_feature (1)..(1): Linked to lys-lys-lys
  misc_feature (5)..(7): n = Pseudoisocytosine
  misc_feature (7)..(8): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (25)..(25): Linked to lys-lys-lys
ttttnnncccttttgctaatcatgt

SEQ ID NO: 18 - DNA, 24 bp, Artificial Sequence
  Synthetic Peptide Nucleic Acid
  misc_feature (1)..(1): Linked to lys-lys-lys
  misc_feature (4)..(4): n = Pseudoisocytosine
  misc_feature (6)..(7): n = Pseudoisocytosine
  misc_feature (7)..(8): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (24)..(24): Linked to lys-lys-lys
tttntnncctctttgcaccattct

SEQ ID NO: 19 - DNA, 10 bp, Artificial Sequence
  Synthetic Primer
  misc_feature (2)..(2): n = Pseudoisocytosine
  misc_feature (7)..(7): n = Pseudoisocytosine
  misc_feature (10)..(10): n = Pseudoisocytosine
tnttttnttn

SEQ ID NO: 20 - DNA, 10 bp, Artificial Sequence
  Synthetic Primer
cttcttttct

SEQ ID NO: 21 - DNA, 10 bp, Artificial Sequence
  Synthetic Primer
  misc_feature (3)..(3): n = Pseudoisocytosine
  misc_feature (6)..(6): n = Pseudoisocytosine
  misc_feature (10)..(10): n = Pseudoisocytosine
ttnttntttn

SEQ ID NO: 22 - DNA, 10 bp, Artificial Sequence
  Synthetic Primer
ctttcttctt

SEQ ID NO: 23 - DNA, 10 bp, Artificial Sequence
  Synthetic Primer
  misc_feature (1)..(3): n = Pseudoisocytosine
  misc_feature (5)..(6): n = Pseudoisocytosine
  misc_feature (9)..(9): n = Pseudoisocytosine
nnntnnttnt

SEQ ID NO: 24 - DNA, 10 bp, Artificial Sequence
  Synthetic Primer
tcttcctccc

SEQ ID NO: 25 - DNA, 20 bp, Artificial Sequence
  Synthetic Peptide Nucleic Acid
  misc_feature (1)..(1): Linked to lys-lys-lys
  misc_feature (2)..(2): n = Pseudoisocytosine
  misc_feature (7)..(7): n = Pseudoisocytosine
  misc_feature (10)..(10): n = Pseudoisocytosine
  misc_feature (10)..(11): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (20)..(20): Linked to lys-lys-lys
tnttttnttncttcttttct

SEQ ID NO: 26 - DNA, 20 bp, Artificial Sequence
  Synthetic Peptide Nucleic Acid
  misc_feature (1)..(1): Linked to lys-lys-lys
  misc_feature (3)..(3): n = Pseudoisocytosine
  misc_feature (6)..(6): n = Pseudoisocytosine
  misc_feature (10)..(10): n = Pseudoisocytosine
  misc_feature (10)..(11): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (20)..(20): Linked to lys-lys-lys
ttnttntttnctttcttctt

SEQ ID NO: 27 - DNA, 20 bp, Artificial Sequence
  Synthetic Peptide Nucleic Acid
  misc_feature (1)..(1): Linked to lys-lys-lys
  misc_feature (1)..(3): n = Pseudoisocytosine
  misc_feature (5)..(6): n = Pseudoisocytosine
  misc_feature (9)..(9): n = Pseudoisocytosine
  misc_feature (10)..(11): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (20)..(20): Linked to lys-lys-lys
nnntnnttnttcttcctccc

SEQ ID NO: 28 - DNA, 15 bp, Artificial Sequence
  Synthetic Primer
cttctccaaaggagt

SEQ ID NO: 29 - DNA, 18 bp, Artificial Sequence
  Synthetic Primer
cttctccacaggagtcag

SEQ ID NO: 30 - DNA, 22 bp, Artificial Sequence
  synthetic polynucleotide
cttctccacaggagtcaggtgc

SEQ ID NO: 31 - DNA, 15 bp, Artificial Sequence
  Synthetic Primer
tctccttaaacctgt

SEQ ID NO: 32 - DNA, 18 bp, Artificial Sequence
  synthetic polynucleotide
tctccttaaacctgtctt

SEQ ID NO: 33 - DNA, 16 bp, Artificial Sequence
  Synthetic Primer
tcttctctgtctccac

SEQ ID NO: 34 - DNA, 18 bp, Artificial Sequence
  Synthetic Primer
tcttctctgtctccacat

SEQ ID NO: 35 - DNA, 22 bp, Artificial Sequence
  Synthetic Peptide Nucleic Acid
  misc_feature (1)..(1): Linked to lys-lys-lys
  misc_feature (1)..(2): n = Pseudoisocytosine
  misc_feature (4)..(4): n = Pseudoisocytosine
  misc_feature (7)..(7): n = Pseudoisocytosine
  misc_feature (7)..(8): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (22)..(22): Linked to lys-lys-lys
nntnttncttctccaaaggagt

SEQ ID NO: 36 - DNA, 22 bp, Artificial Sequence
  Synthetic Peptide Nucleic Acid
  misc_feature (1)..(1): Linked to lys-lys-lys
  misc_feature (3)..(4): n = Pseudoisocytosine
  misc_feature (6)..(6): n = Pseudoisocytosine
  misc_feature (7)..(8): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (22)..(22): Linked to lys-lys-lys
ttnntnttctccttaaacctgt

SEQ ID NO: 37 - DNA, 25 bp, Artificial Sequence
  Synthetic Peptide Nucleic Acid
  misc_feature (1)..(1): Linked to lys-lys-lys
  misc_feature (3)..(4): n = Pseudoisocytosine
  misc_feature (6)..(6): n = Pseudoisocytosine
  misc_feature (7)..(8): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (25)..(25): Linked to lys-lys-lys
ttnntnttctccttaaacctgtctt

SEQ ID NO: 38 - DNA, 24 bp, Artificial Sequence
  Synthetic Peptide Nucleic Acid
  misc_feature (1)..(1): Linked to lys-lys-lys
  misc_feature (2)..(2): n = Pseudoisocytosine
  misc_feature (4)..(4): n = Pseudoisocytosine
  misc_feature (7)..(7): n = Pseudoisocytosine
  misc_feature (8)..(9): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (24)..(24): Linked to lys-lys-lys
tntnttnttcttctctgtctccac

SEQ ID NO: 39 - DNA, 25 bp, Artificial Sequence
  Synthetic Peptide Nucleic Acid
  misc_feature (1)..(1): Linked to lys-lys-lys
  misc_feature (1)..(2): n = Pseudoisocytosine
  misc_feature (4)..(4): n = Pseudoisocytosine
  misc_feature (7)..(7): n = Pseudoisocytosine
  misc_feature (7)..(8): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (25)..(25): Linked to lys-lys-lys
nntnttncttctccacaggagtcag

SEQ ID NO: 40 - DNA, 29 bp, Artificial Sequence
  Synthetic Peptide Nucleic Acid
  misc_feature (1)..(1): Linked to lys-lys-lys
  misc_feature (1)..(2): n = Pseudoisocytosine
  misc_feature (4)..(4): n = Pseudoisocytosine
  misc_feature (7)..(7): n = Pseudoisocytosine
  misc_feature (7)..(8): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (29)..(29): Linked to lys-lys-lys
nntnttncttctccacaggagtcaggtgc

SEQ ID NO: 41 - DNA, 26 bp, Artificial Sequence
  Synthetic Peptide Nucleic Acid
  misc_feature (1)..(1): Linked to lys-lys-lys
  misc_feature (2)..(2): n = Pseudoisocytosine
  misc_feature (4)..(4): n = Pseudoisocytosine
  misc_feature (7)..(7): n = Pseudoisocytosine
  misc_feature (8)..(9): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (26)..(26): Linked to lys-lys-lys
tntnttnttcttctctgtctccacat

SEQ ID NO: 42 - DNA, 51 bp, Artificial Sequence
  Synthetic Primer
gttcagcgtgtccggcgagggcgaggtgagtctatgggacccttgatgttt

SEQ ID NO: 43 - DNA, 51 bp, Artificial Sequence
  Synthetic Primer
aaacatcaagggtcccatagactcacctcgccctcgccggacacgctgaac

SEQ ID NO: 44 - DNA, 70 bp, Artificial Sequence
  Synthetic Primer
cttgccccacagggcagtaacggcagatttttcttccggcgttaaatgcaccatggtgtctgtttgaggt

SEQ ID NO: 45 - DNA, 51 bp, Artificial Sequence
  Synthetic Primer
acagacaccatggtgcacctgactcctgaggagaagtctgccgttactgcc

SEQ ID NO: 46 - DNA, 65 bp, Artificial Sequence
  Synthetic Primer
  misc_feature (1)..(2): Optional phosphorothioate internucleoside linkage
  misc_feature (2)..(3): Optional phosphorothioate internucleoside linkage
  misc_feature (3)..(4): Optional phosphorothioate internucleoside linkage
  misc_feature (62)..(63): Optional phosphorothioate internucleoside linkage
  misc_feature (63)..(64): Optional phosphorothioate internucleoside linkage
  misc_feature (64)..(65): Optional phosphorothioate internucleoside linkage
ttgccccacagggcagtaacggcagacttctcctcaggagtcaggtgcaccatggtgtctgtttg

SEQ ID NO: 47 - DNA, 17 bp, Artificial Sequence
  Synthetic Primer
tttcctctatgggtaag

SEQ ID NO: 48 - DNA, 17 bp, Artificial Sequence
  Synthetic Primer
cttacccatagaggaaa

SEQ ID NO: 49 - DNA, 17 bp, Artificial Sequence
  Synthetic Primer
atgccaactagaagagg

SEQ ID NO: 50 - DNA, 17 bp, Artificial Sequence
  Synthetic Primer
cctcttctagttggcat

SEQ ID NO: 51 - DNA, 18 bp, Artificial Sequence
  Synthetic Primer
ctttcccttgtatctttt

SEQ ID NO: 52 - DNA, 18 bp, Artificial Sequence
  Synthetic Primer
aaaagatacaagggaaag

SEQ ID NO: 53 - DNA, 10 bp, Artificial Sequence
  Synthetic Primer
gaaggagaaa

SEQ ID NO: 54 - DNA, 27 bp, Artificial Sequence
  Synthetic Peptide Nucleic Acid
  misc_feature (1)..(1): Linked to lys-lys-lys
  misc_feature (3)..(5): n = Pseudoisocytosine
  misc_feature (9)..(9): n = Pseudoisocytosine
  misc_feature (9)..(10): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (27)..(27): Linked to lys-lys-lys
ttnnntttnctttcccttgtatctttt

SEQ ID NO: 55 - DNA, 10 bp, Artificial Sequence
  Synthetic Primer
agaaaaaagg

SEQ ID NO: 56 - DNA, 10 bp, Artificial Sequence
  Synthetic Primer
cttcctcttt

SEQ ID NO: 57 - DNA, 10 bp, Artificial Sequence
  Synthetic Primer
tttctccttc

SEQ ID NO: 58 - DNA, 18 bp, Artificial Sequence
  Synthetic Primer
tttctccttcagtgttca

SEQ ID NO: 59 - DNA, 20 bp, Artificial Sequence
  Synthetic Primer
tccttttgctcacctgtggt

SEQ ID NO: 60 - DNA, 10 bp, Artificial Sequence
  Synthetic Primer
tcttttttcc

SEQ ID NO: 61 - DNA, 10 bp, Artificial Sequence
  Synthetic Primer
ccttttttct

SEQ ID NO: 62 - DNA, 18 bp, Artificial Sequence
  Synthetic Primer
ccttttttctggctaagt

SEQ ID NO: 63 - DNA, 10 bp, Artificial Sequence
  Synthetic Primer
  misc_feature (1)..(1): n = Pseudoisocytosine
  misc_feature (4)..(5): n = Pseudoisocytosine
  misc_feature (7)..(7): n = Pseudoisocytosine
nttnntnttt

SEQ ID NO: 64 - DNA, 10 bp, Artificial Sequence
  Synthetic Primer
  misc_feature (2)..(2): n = Pseudoisocytosine
  misc_feature (9)..(10): n = Pseudoisocytosine
tnttttttnn

SEQ ID NO: 65 - DNA, 28 bp, Artificial Sequence
  Synthetic Peptide Nucleic Acid
  misc_feature (1)..(1): Linked to lys-lys-lys
  misc_feature (1)..(1): n = Pseudoisocytosine
  misc_feature (4)..(5): n = Pseudoisocytosine
  misc_feature (7)..(7): n = Pseudoisocytosine
  misc_feature (10)..(11): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (29)..(29): Linked to lys-lys-lys
nttnntnttttttctccttcagtgttca

SEQ ID NO: 66 - DNA, 27 bp, Artificial Sequence
  Synthetic Peptide Nucleic Acid
  misc_feature (1)..(1): Linked to lys-lys-lys
  misc_feature (5)..(6): n = Pseudoisocytosine
  misc_feature (7)..(8): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (27)..(27): Linked to lys-lys-lys
ttttnnttccttttgctcacctgtggt

SEQ ID NO: 67 - DNA, 28 bp, Artificial Sequence
  Synthetic Peptide Nucleic Acid
  misc_feature (1)..(1): Linked to lys-lys-lys
  misc_feature (2)..(2): n = Pseudoisocytosine
  misc_feature (9)..(10): n = Pseudoisocytosine
  misc_feature (10)..(11): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (28)..(28): Linked to lys-lys-lys
tnttttttnnccttttttctggctaagt

SEQ ID NO: 68 - DNA, 61 bp, Artificial Sequence
  Synthetic Primer
  misc_feature (1)..(2): Optional phosphorothioate internucleoside linkage
  misc_feature (2)..(3): Optional phosphorothioate internucleoside linkage
  misc_feature (3)..(4): Optional phosphorothioate internucleoside linkage
  misc_feature (58)..(59): Optional phosphorothioate internucleoside linkage
  misc_feature (59)..(60): Optional phosphorothioate internucleoside linkage
  misc_feature (60)..(61): Optional phosphorothioate internucleoside linkage
tcttgggattcaataaccttgcagacagtggaggaaggcctttggcgtgataccacaggtg

SEQ ID NO: 69 - DNA, 18 bp, Artificial Sequence
  Synthetic Primer
tttttctgtaatttttaa

SEQ ID NO: 70 - DNA, 17 bp, Artificial Sequence
  Synthetic Primer
tctttctctgcaaactt

SEQ ID NO: 71 - DNA, 18 bp, Artificial Sequence
  Synthetic Primer
tttctttaagaacgagca

SEQ ID NO: 72 - DNA, 25 bp, Artificial Sequence
  Synthetic Peptide Nucleic Acid
  misc_feature (1)..(1): Linked to lys-lys-lys
  misc_feature (2)..(2): n = Pseudoisocytosine
  misc_feature (7)..(8): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (25)..(25): Linked to lys-lys-lys
tnttttttttttctgtaatttttaa

SEQ ID NO: 73 - DNA, 26 bp, Artificial Sequence
  Synthetic Peptide Nucleic Acid
  misc_feature (1)..(1): Linked to lys-lys-lys
  misc_feature (2)..(2): n = Pseudoisocytosine
  misc_feature (4)..(4): n = Pseudoisocytosine
  misc_feature (8)..(8): n = Pseudoisocytosine
  misc_feature (9)..(10): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (26)..(26): Linked to lys-lys-lys
tntntttnttctttctctgcaaactt

SEQ ID NO: 74 - DNA, 25 bp, Artificial Sequence
  Synthetic Peptide Nucleic Acid
  misc_feature (1)..(1): Linked to lys-lys-lys
  misc_feature (4)..(4): n = Pseudoisocytosine
  misc_feature (7)..(8): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (25)..(25): Linked to lys-lys-lys
tttnttttttctttaagaacgagca

SEQ ID NO: 75 - DNA, 63 bp, Artificial Sequence
  Synthetic Primer
  misc_feature (1)..(2): Optional phosphorothioate internucleoside linkage
  misc_feature (2)..(3): Optional phosphorothioate internucleoside linkage
  misc_feature (3)..(4): Optional phosphorothioate internucleoside linkage
  misc_feature (60)..(61): Optional phosphorothioate internucleoside linkage
  misc_feature (61)..(62): Optional phosphorothioate internucleoside linkage
  misc_feature (62)..(63): Optional phosphorothioate internucleoside linkage
tccaagtttgcagagaaagataatatagtccttggagaaggaggaatcaccctgagtggaggt

SEQ ID NO: 76 - DNA, 10 bp, Artificial Sequence
  Synthetic Primer
ctcttcttct

SEQ ID NO: 77 - DNA, 10 bp, Artificial Sequence
  Synthetic Primer
tcttcttctc

SEQ ID NO: 78 - DNA, 15 bp, Artificial Sequence
  Synthetic Primer
tcttcttctcatttc

SEQ ID NO: 79 - DNA, 25 bp, Artificial Sequence
  Synthetic Peptide Nucleic Acid
  misc_feature (1)..(1): Linked to lys-lys-lys
  misc_feature (1)..(1): n = Pseudoisocytosine
  misc_feature (3)..(3): n = Pseudoisocytosine
  misc_feature (6)..(6): n = Pseudoisocytosine
  misc_feature (9)..(9): n = Pseudoisocytosine
  misc_feature (10)..(11): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (25)..(25): Linked to lys-lys-lys
ntnttnttnttcttcttctcatttc

SEQ ID NO: 80 - DNA, 20 bp, Artificial Sequence
  Synthetic Peptide Nucleic Acid
  misc_feature (1)..(1): Linked to lys-lys-lys
  misc_feature (1)..(1): n = Pseudoisocytosine
  misc_feature (4)..(4): n = Pseudoisocytosine
  misc_feature (5)..(6): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (20)..(20): Linked to lys-lys-lys
nttnttcttcttctcatttc

SEQ ID NO: 81 - DNA, 60 bp, Artificial Sequence
  Synthetic Primer
attcccgagtagcagatgaccatgacagcttagggcaggaccagccccaagatgactatc

SEQ ID NO: 82 - DNA, 60 bp, Artificial Sequence
  Synthetic Primer
tttaggattcccgagtagcagatgacccctcagagcagcggcaggaccagccccaagatg

SEQ ID NO: 83 - DNA, 67 bp, Artificial Sequence
  Synthetic Primer
gggacggcgcccacataggccaaattcaattgctgatcccagcttaagacgtactggtcagcctggc

SEQ ID NO: 84 - DNA, 10 bp, Artificial Sequence
  Synthetic Primer
  misc_feature (1)..(1): n = Pseudoisocytosine
  misc_feature (3)..(3): n = Pseudoisocytosine
  misc_feature (6)..(6): n = Pseudoisocytosine
  misc_feature (9)..(9): n = Pseudoisocytosine
ntnttnttnt

SEQ ID NO: 85 - DNA, 12 bp, Artificial Sequence
  Synthetic Primer
  misc_feature (1)..(1): n = Pseudoisocytosine
  misc_feature (5)..(5): n = Pseudoisocytosine
  misc_feature (9)..(9): n = Pseudoisocytosine
  misc_feature (11)..(11): n = Pseudoisocytosine
ntttntttntnt

SEQ ID NO: 86 - DNA, 60 bp, Artificial Sequence
  Synthetic Primer
aaagaataacagtgataatttctgggttaaggcaatagcaatatctctgcatataaatat

SEQ ID NO: 87 - DNA, 12 bp, Artificial Sequence
  Synthetic Primer
ctgctcggaaga

SEQ ID NO: 88 - DNA, 15 bp, Artificial Sequence
  Synthetic Primer
ccttcaccaagggga

SEQ ID NO: 89 - DNA, 25 bp, Artificial Sequence
  Synthetic Peptide Nucleic Acid
  misc_feature (1)..(1): Linked to lys-lys-lys
  misc_feature (2)..(2): n = Pseudoisocytosine
  misc_feature (4)..(5): n = Pseudoisocytosine
  misc_feature (8)..(9): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (25)..(25): Linked to lys-lys-lys
tntnnttttttcctctatgggtaag

SEQ ID NO: 90 - DNA, 25 bp, Artificial Sequence
  Synthetic Peptide Nucleic Acid
  misc_feature (1)..(1): Linked to lys-lys-lys
  misc_feature (2)..(2): n = Pseudoisocytosine
  misc_feature (5)..(5): n = Pseudoisocytosine
  misc_feature (7)..(8): n = Pseudoisocytosine
  misc_feature (8)..(9): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (25)..(25): Linked to lys-lys-lys
tnttntnncctcttctagttggcat

SEQ ID NO: 91 - DNA, 61 bp, Artificial Sequence
  Synthetic Primer
ttctgtatctatattcatcataggaaacaccaaagataatgttctccttaatggtgccagg

SEQ ID NO: 92 - DNA, 65 bp, Artificial Sequence
  Synthetic Primer
gatgactatctttaatgtctggaaattcttccagaattaattaagactgtatggaaaatgagagc

SEQ ID NO: 93 - DNA, 66 bp, Artificial Sequence
  Synthetic Primer
ccccaagatgactatctttaatgtctggaacgatcatcagaattgatactgactgtatggaaaatg

SEQ ID NO: 94 - DNA, 65 bp, Artificial Sequence
  Synthetic Primer
gatgactatctttaatgtctggaaattctactagaattgatactgactgtatggaaaatgagagc

SEQ ID NO: 95 - DNA, 15 bp, Artificial Sequence
  Synthetic Primer
tccccttggtgaagg

SEQ ID NO: 96 - DNA, 22 bp, Artificial Sequence
  Synthetic Peptide Nucleic Acid
  misc_feature (1)..(1): Linked to lys-lys-lys
  misc_feature (3)..(6): n = Pseudoisocytosine
  misc_feature (7)..(8): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (22)..(22): Linked to lys-lys-lys
ttnnnnttccccttggtgaagg

SEQ ID NO: 97 - DNA, 63 bp, Artificial Sequence
  Synthetic PRimer
aggacggtcccggcctgcgacacttccgcccataattgttcttcatctgcggggcgggggggg

SEQ ID NO: 98 - DNA, 12 bp, Artificial Sequence
  Synthetic Primer
tcttccgagcag

SEQ ID NO: 99 - DNA, 18 bp, Artificial Sequence
  Synthetic Primer
  misc_feature (1)..(1): Linked to lys-lys-lys
  misc_feature (1)..(2): n = Pseudoisocytosine
  misc_feature (5)..(5): n = Pseudoisocytosine
  misc_feature (6)..(7): Linked by three 8-amino-2, 6, 10-trioxaoctanoic acid, three 8-amino-3,6-dioxaoctanoic acid, three 6-aminohexanoic acid , or three 11-amino-3,6,9-trioxaundecanoic acid molecules
  misc_feature (18)..(18): Linked to lys-lys-lys
nnttnttcttccgagcag